(19) United States
(12) Patent Application Publication
(10) Pub. No.: US 2025/0019700 A1
(43) Pub. Date: Jan. 16, 2025

(54) TREATING CHEMORESISTANT FORMS OF LEUKEMIA AND OTHER CANCERS THAT OVEREXPRESS RNA BINDING PROTEIN FXR1

(71) Applicant: The General Hospital Corporation, Boston, MA (US)

(72) Inventors: Shobha VASUDEVAN, Boston, MA (US); Chandreyee DATTA, Boston, MA (US); Samuel Spencer TRUESDELL, Boston, MA (US)

(21) Appl. No.: 18/707,399

(22) PCT Filed: Nov. 8, 2022
(86) PCT No.: PCT/US2022/079489

§ 371 Date: May 3, 2024

Related U.S. Application Data
(60) Provisional application No. 63/276,886, filed on Nov. 8, 2021.
(60) Provisional application No. 63/420,478, filed on Oct. 28, 2022.

(51) Int. Cl. C12N 15/113

(52) U.S. Cl. CPC C12N 15/113; C12N 2310/11; C12N 2310/531

(57) ABSTRACT

The invention features compositions and methods useful for targeting and activating translation, for example, non-canonical translation, in cancer cells.

### CROSS-REFERENCE TO RELATED APPLICATIONS

This application claims benefit of 63/276,886, filed Nov. 8, 2021, and 63/420,478, filed Oct. 28, 2022, each of which are hereby incorporated by reference in their entirety.

### SEQUENCE LISTING

The instant application contains a Sequence Listing which has been submitted electronically in XML format and is hereby incorporated by reference in its entirety. Said XML copy, created on Nov. 7, 2022, is named 51536-004WO4_Sequence_Listing_11_7_22.xml and is 107,082 bytes in size.

### BACKGROUND OF THE INVENTION

This invention relates to a method of treating chemo-resistant cancers by administering inhibitors of a stress pathway that is activated by fragile-X-mental retardation related protein (FXR1) dysregulation and FXR1-dependent changes in (a) non-canonical translation of specific pro-survival genes and (b) ribosome biogenesis.

Cancer cells can enter a reversible arrest phase called quiescence, or G0, which is resistant to harsh conditions including chemotherapy. FXR1 increases in serum-starved G0 acute monocytic leukemic (AML) cells. FXR1 is important for tumor progression as it is amplified in several aggressive cancers, where post-transcriptional expression of specific mRNAs is altered. FXR1 is associated with translation, mRNA stability and localization, in the nucleus, cytoplasm and stress granules. Canonical translation inhibition causes a switch to specialized non-canonical mechanisms in serum-starved G0 cells, where FXR1 binds and promotes specific mRNA translation. Given that FXR1 increases in serum-starved G0 cells and in aggressive cancers, and promotes specific translation in G0 cells that are chemoresistant, the impact of FXR1 on chemosurvival via translation mechanisms needs to be uncovered.

### SUMMARY OF THE INVENTION

The invention, in general, features compositions and methods useful for targeting and activating translation, for example, non-canonical translation, in cancer cells.

In one aspect, provided herein is an isolated nucleic acid molecule including a G-rich 5′ untranslated region (UTR) operably linked to a heterologous transgene.

In some embodiments, the 5′-UTR includes motif 2 (SEQ ID NO: 14):

[symbol]

[symbol]

[symbol]

GCR¹CR²R³CR³R³R³R²R³CR³CR³R²R³CR²CCR³CR²GCR²R³CCR²ACCCCR³R³GCR¹CGC wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; and each R³, independently, denotes a guanosine or cytidine nucleoside.

In some embodiments, the 5′-UTR includes motif 2 (SEQ ID NO: 14):

[symbol]

[symbol]

[symbol]

R¹CR²CR³R²R³GR⁴R²CR²GR⁵R²GCR²GCGGCGGCGR³R²GGCTGR²GGCGGCGGCGR⁴R³G wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; each R⁴, independently, denotes a cytidine, guanosine, or adenosine nucleoside; and each R⁵, independently, denotes a cytidine or adenosine nucleoside.

In yet other embodiments the 5′-UTR includes motif 3 (SEQ ID NO: 15):

[symbol]

[symbol]

[symbol]

GCCR³R²R¹GCCGR³CR⁴CCR³CCGCR¹ wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; and each R⁴, independently, denotes a thymidine or guanosine nucleoside.

In some embodiments, the nucleic acid molecule further includes a non-canonical translation start site. In some embodiments, the non-canonical translation start site includes a poor Kozak consensus sequence or a non-AUG start site. For example, in some embodiments, the non-canonical translation start site includes a codon selected from AUA, CUG, GUG, CUG, ACG, and AUC.

In some embodiments, the nucleic acid molecule is RNA. In some embodiments, the RNA is messenger RNA (mRNA).

In some embodiments, the nucleic acid molecule further includes a 3′ untranslated region (UTR).

In some embodiments, the nucleic acid molecule further includes a 3′ poly-adenosine (polyA) tail. In some embodiments, the polyA tail includes from about 10 to about 100 continuous adenosine residues.

In other embodiments, the nucleic acid molecule further includes a 5′ cap.

In some embodiments, the nucleic acid molecule includes a poly-A tail.

In some embodiments, the nucleic acid molecule includes a chemical modification. In some embodiments, the chemical modification includes a modified nucleoside. In some embodiments, the modified nucleoside is N6-methyladenosine.

In other embodiments, the nucleic acid molecule is operably linked to a polypeptide.

In yet other aspects, the invention features a pharmaceutical composition which includes any of the foregoing nucleic acid molecules and one or more pharmaceutically acceptable carriers, diluents, and/or excipients, optionally wherein the pharmaceutical composition includes an exosome, liposome, or lipid nanoparticle (LNP).

In yet another aspect, the invention features an LNP and nucleic acid molecules described herein.

In another aspect, provided herein is an antisense oligonucleotide (ASO) molecule targeting a G-rich 5′-UTR.

In some embodiments, the 5′-UTR includes motif 1 (SEQ ID NO: 13):

[symbol]

[symbol]

[symbol]

GCR¹CR²R³CR³R³R³R²R³CR³CR³R²R³CR²CCR³CR²GCR²R³CCR²ACCCCR³R³GCR¹CGC wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; and each R³, independently, denotes a guanosine or cytidine nucleoside.

In some embodiments, the 5′-UTR includes motif 2 (SEQ ID NO: 14)

[symbol]

[symbol]

[symbol]

R¹CR²CR³R²R³GR⁴R²CR²GR⁵R²GCR²GCGGCGGCGR³R²GGCTGR²GGCGGCGGCGR⁴R³G wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; each R⁴, independently, denotes a cytidine, guanosine, or adenosine nucleoside; and each R⁵, independently, denotes a cytidine or adenosine nucleoside.

In some embodiments, the 5′-UTR includes motif 3 (SEQ ID NO: 15):

[symbol]

[symbol]

[symbol]

GCCR³R²R¹GCCGR³CR⁴CCR³CCGCR¹
wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; and each R⁴, independently, denotes a thymidine or guanosine nucleoside.

In other embodiments, the ASO includes a chemical modification. In some embodiments, the chemical modification includes a modified nucleoside. In some embodiments, the modified nucleoside is N6-methyladenosine. In some embodiments, the chemical modification includes, for example, one or more of a 2′-O-methyl (2′-O-Me) modified nucleoside, a phosphorothioate (PS) bond between nucleosides, and a 2′-fluoro (2′-F) modified nucleoside.

In another aspect, the invention features a pharmaceutical composition including the ASO and one or more pharmaceutically acceptable carriers, diluents, and/or excipients, optionally wherein the pharmaceutical composition includes an exosome, liposome, or LNP.

In yet another aspect, provided herein is a composition including an LNP and any of the foregoing ASOs.

In another aspect, the invention features a synthetic, G-rich 5′-UTR. In some embodiments, the 5′-UTR is between 15 and 50 nucleotides in length. In some embodiments, the 5′-UTR includes a chemical modification. In some embodiments, the chemical modification includes a modified nucleoside. In some embodiments, the modified nucleoside is N6-methyladenosine. In some embodiments, the chemical modification includes, for example, one or more of a 2′-O-Me modified nucleoside, a PS bond between nucleosides, and a 2′-F modified nucleoside.

In some embodiments, the 5′-UTR includes motif 1 (SEQ ID NO: 13):

[symbol]

[symbol]

[symbol]

[symbol]

GCR¹CR²R³CR³R³R³R²R³CR³CR³R²R³CR²CCR³CR²GCR²R³CCR²ACCCCR³R³GCR¹CGC
wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; and each R³, independently, denotes a guanosine or cytidine nucleoside.

In some embodiments, the 5′-UTR includes motif 2 (SEQ ID NO: 14):

[symbol]

[symbol]

[symbol]

[symbol]

R¹CR²CR³R²R³GR⁴R²CR²GR⁵R²GCR²GCGGCGGCGR³R²GGCTGR²GGCGGCGGCGR⁴R³G
wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; each R⁴, independently, denotes a cytidine, guanosine, or adenosine nucleoside; and each R⁵, independently, denotes a cytidine or adenosine nucleoside.

In some embodiments, the 5′-UTR includes motif 3 (SEQ ID NO: 15):

[symbol]

[symbol]

[symbol]

[symbol]

GCCR³R²R¹GCCGR³CR⁴CCR³CCGCR¹
wherein each G denotes a guanosine nucleoside; each C denotes a cytidine nucleoside; each A denotes an adenosine nucleoside; each R¹, independently, denotes a cytidine or thymidine nucleoside; each R², independently, denotes a cytidine, thymidine, guanosine, or adenosine nucleoside; each R³, independently, denotes a guanosine or cytidine nucleoside; and each R⁴, independently, denotes a thymidine or guanosine nucleoside.

In another aspect, provided herein is an LNP including a G-rich 5′-UTR, optionally wherein the 5′-UTR is any of the foregoing 5′-UTRs.

In another aspect, the invention features an engineered cell including any of the foregoing nucleic acid molecules, ASOs, or 5′-UTRs.

In yet another aspect, provided herein is a treatment method including the step of: transfecting a cell, in a subject, with any of the foregoing nucleic acid molecules, ASOs, or 5′-UTRs in an amount effective to treat the subject.

In still another aspect, the invention features a composition including any of the foregoing engineered cells. In some embodiments, the composition is a pharmaceutical composition including one or more pharmaceutically acceptable carriers, diluents, or excipients.

In yet another aspect, provided herein is a method of administering a 5′-UTR to a subject to treat a disease or disorder, the method including: administering to the subject a therapeutically effective amount of the composition of any one of the foregoing aspects. In some embodiments, the composition is formulated as an extracellular vesicle, a liposome, or an LNP.

In another aspect, the invention features a method of delivering a 5′-UTR to a cell, the method including: a. transfecting the cell with any of the foregoing nucleic acid molecules, ASOs, or 5′-UTRs; and, optionally, b. culturing the cell in vitro; thereby delivering the 5′-UTR to the cell.

In another aspect, provided herein is a method of treating a leukemia in a subject including administering to the subject a therapeutically effective dose of an inhibitor of fragile X mental retardation-related protein 1 (FXR1) alone or in combination with a chemotherapeutic, thereby treating leukemia in the subject. In some embodiments, the leukemia is resistant to one or more chemotherapeutics. In some embodiments, the leukemia expresses FXR1.

In some embodiments, the subject has previously been treated with one or more chemotherapeutics.

In some embodiments, the leukemia is acute myeloid leukemia (AML). In some embodiments, the leukemia is chronic myeloid leukemia (CML), acute lymphocytic leukemia (ALL), or chronic lymphocytic leukemia (CLL).

In some embodiments, the chemotherapeutic is cytarabine, doxorubicin, etoposide, gemcitabine, paclitaxel, and taxol. In some embodiments, the chemotherapeutic is administered concurrently with the FXR1 inhibitor. In some embodiments, the FXR1 inhibitor is administered after the chemotherapeutic. In some embodiments, the FXR1 inhibitor is, for example, a BCL6 inhibitor, a XBP1 inhibitor, a ISR inhibitor, a PI3K/mTOR or RPS6K inhibitor, a PDK inhibitor, a PKR inhibitor, a GCN2 inhibitor, a CD47 inhibitor, or a shRNA. In some embodiments, the BCL6 inhibitor is FX1. In some embodiments, the XBP1 inhibitor is toyocamycin. In some embodiments, the ISR inhibitor is selected from ISRIB and metformin. In some embodiments, the P13K/mTOR or RPS6K inhibitor is selected from bez235, torin, pp242, AZD3147, everolimus, CAL-101 and LY2584702. In some embodiments, the PDK inhibitor is AZD7545. In some embodiments, the PKR inhibitor is c16. In some embodiments, the GCN2 inhibitor is, for example, GZD824, A-92, or a triazolo[4,5-d]pyrimidine. In some embodiments, the CD47 inhibitor is a CD47 antibody including Magrolimab, CC-90002, and IB1188, or an siRNA sequence selected from:

(SEQ ID NO: 6)
5-TACTGGCCTTGGTTTAATTCTCGAGAATTAAACCAAGGCCAGTAGC-3
(SEQ ID NO: 7)
5-CUAUGAGACCCUUACGUGAUUGUUA-3
(SEQ ID NO: 8)
5-GCACAUGCAUCUUCUGUAUGGACAA-3
(SEQ ID NO: 9)
5-GCAUGGCCCUCUUCUGAUU-3
(SEQ ID NO: 10)
5-GUACAGCGAUUGGAUUAAC-3
(SEQ ID NO: 11)
5-CAGAGAAGGUGAAACGAUC-3,
and
(SEQ ID NO: 12)
5-UAACUGAAGUGAAGUGAUG-3.

In some embodiments, the shRNA inhibits eIF2α activation. In some embodiments, the shRNA is an antisense sequence selected from:

(SEQ ID NO: 1)
ATCCGTCTCCACAGACAAG;
(SEQ ID NO: 2)
TGTTTTGCAAATAAAACTG;
(SEQ ID NO: 3)
TGAGCATGTTCAGCAGCGT;
(SEQ ID NO: 4)
TGGACTTCCAGGTCGCCCT;
and
(SEQ ID NO: 5)
TGCGCATCATGGTGTTCTT.

In yet another aspect, the invention features a method of treating a chemoresistant cancer in a subject including administering to the subject a therapeutically effective dose of an inhibitor of FXR1 alone or in combination with a chemotherapeutic, thereby treating chemoresistant cancer in the subject. In some embodiments, the chemoresistant cancer expresses FXR1. In some embodiments, the chemoresistant cancer is selected from, for example, a leukemia, a lung cancer, a breast cancer, an ovarian cancer, a head and neck cancer, an esophageal cancer, a cervical cancer, a uterine cancer, a stomach cancer, a bladder cancer, a prostate cancer, a urothelial cancer, a pancreatic cancer, or a brain cancer.

In some embodiments, the subject has previously been treated with one or more chemotherapeutics.

In some embodiments, the chemotherapeutic is cytarabine, doxorubicin, etoposide, gemcitabine, paclitaxel, and taxol. In some embodiments, the chemotherapeutic is administered concurrently with the FXR1 inhibitor. In some embodiments, the FXR1 inhibitor is administered after the chemotherapeutic. In some embodiments, the FXR1 inhibitor is a BCL6 inhibitor, a XBP1 inhibitor, a ISR inhibitor, a PI3K/mTOR inhibitor, a PDK inhibitor, a PKR inhibitor, a GCN2 inhibitor, a CD47 inhibitor, or a shRNA. In some embodiments, the BCL6 inhibitor is FX1. In some embodiments, the XBP1 inhibitor is toyocamycin. In some embodiments, the ISR inhibitor is selected from ISRIB and metformin. In some embodiments, the P13K/mTOR inhibitor or RPS6K inhibitor is selected from bez235, torin, pp242, AZD3147, everolimus, CAL-101 and LY2584702. In some embodiments, the PDK inhibitor is AZD7545. In some embodiments, the PKR inhibitor is c16. In some embodiments, the GCN2 inhibitor is GZD824, A-92, or a triazolo[4,5-d]pyrimidine. In some embodiments, the CD47 inhibitor is a CD47 antibody including Magrolimab, CC-90002, and IB1188, or an siRNA sequence selected from:

(SEQ ID NO: 6)
5-TACTGGCCTTGGTTTAATTCTCGAGAATTAAACCAAGGCCAGTAGC-3
(SEQ ID NO: 7)
5-CUAUGAGACCCUUACGUGAUUGUUA-3
(SEQ ID NO: 8)
5-GCACAUGCAUCUUCUGUAUGGACAA-3
(SEQ ID NO: 9)
5-GCAUGGCCCUCUUCUGAUU-3
(SEQ ID NO: 10)
5-GUACAGCGAUUGGAUUAAC-3
(SEQ ID NO: 11)
5-CAGAGAAGGUGAAACGAUC-3,
and
(SEQ ID NO: 12)
5-UAACUGAAGUGAAGUGAUG-3.

In some embodiments, the shRNA inhibits eIF2α activation. In some embodiments, the shRNA is an antisense sequence selected from:

(SEQ ID NO: 1)
ATCCGTCTCCACAGACAAG;
(SEQ ID NO: 2)
TGTTTTGCAAATAAAACTG;
(SEQ ID NO: 3)
TGAGCATGTTCAGCAGCGT;
(SEQ ID NO: 4)
TGGACTTCCAGGTCGCCCT;
and
(SEQ ID NO: 5)
TGCGCATCATGGTGTTCTT.

Other features and advantages of the invention will be apparent from the following description of the preferred embodiments thereof, and from the claims.

### Definitions

As used herein, the term “antisense oligonucleotide” or “ASO” refers to an antisense compound that is an oligonucleotide. An exemplary ASO refers to an inhibitory polynucleotide capable of hybridizing through complementary base-pairing with a target mRNA molecule (e.g., a 5′-UTR or a non-canonical start site, or both, as described herein) and inhibiting its expression through mRNA destabilization and degradation, or inhibition of translation.

As used herein, the term “chemoresistant” refers to a cancer cell or a cancer that fails to respond to treatment by conventional chemotherapeutic agents (e.g., cytarabine, doxorubicin, etoposide, gemcitabine, paclitaxel, and taxol). Chemoresistance may be characterized by recurrence or continuation of cancer growth following a treatment regimen with chemotherapeutics. Chemoresistant cancer cells or cancers may be chemoresistant as a result of, for example, an alteration in cellular transcriptional programs that control cell metabolism, resistance to stress, epithelial-to-mesenchymal transition, and cell cycle regulation, among others.

As used herein, the term “therapeutically effective amount” of a composition described herein refers to a quantity sufficient to, when administered to the subject, including a mammal, for example a human, effect beneficial or desired results, including clinical results, and, as such, an effective amount or synonym thereto depends upon the context in which it is being applied. For example, in the context of treating a cancer, it is an amount of the composition sufficient to achieve a treatment response as compared to the response obtained without administration of the composition. The amount of a given composition described herein that will correspond to such an amount will vary depending upon various factors, such as the given inhibitory agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition of the present disclosure is an amount which results in a beneficial or desired result in a subject as compared to a control. As defined herein, a therapeutically effective amount of a composition of the present disclosure may be readily determined by one of ordinary skill by routine methods known in the art. Dosage regimen may be adjusted to provide the optimum therapeutic response.

As used herein, the term “inhibitor” refers to an agent (e.g., a small molecule (e.g., metformin, phenformin, 8-azaadenosine, enzastaurin, or talazoparib)) peptide fragment, protein, antibody, antigen-binding fragment thereof, or a nucleic acid (e.g., an interfering RNA molecule, such as an antisense RNA, a small hairpin RNA or a small interfering RNA)) that binds to, and/or otherwise suppresses the activity of, a target molecule. Inhibitory agents may reduce expression of a target molecule by 10% or more (e.g., 10%, 20%, 30%, 40%, 50%, 60%, 70%, 80%, 90%, 95%, 98%, 99% or more). In some embodiments, inhibitors of the disclosure include inhibitors of FXR1, including inhibitors of BCL6 (e.g., FX1), inhibitors of XBP1 (e.g., toyocamycin), inhibitors of ISR (e.g., ISRIB and metformin), inhibitors of PI3K/mTOR or RPS6K (e.g., bex235, torin, pp242, AZD3147, everolimus, CAL-101, and LY2584702), inhibitors of PDK (e.g., AZD7545), inhibitors of PKR (e.g., c16), inhibitors of GCN2 (e.g., GZD824, A-92, or a triazolo[4,5-d]pyrimidine), and inhibitors of CD47 (e.g., siRNAs (e.g., an siRNA molecule with the sequence of any one of SEQ ID NOs: 6-12) or CD47 antibodies (e.g., Magrolimab (Selleckchem), CC-90002 (Celgene), and IB1188 (Innovent Biologics)). In some embodiments, the FXR1 inhibitor is an shRNA molecule (e.g., an shRNA molecule having the sequence of any one of SEQ ID NOs: 1-5).

As used herein, the term “integrated stress response” or “ISR” refers to the common adaptive pathway that eukaryotic cells activate in response to stress stimuli. The ISR involves the phosphorylation of eukaryotic translation initiation factor 2 alpha (eIF2α) by members of the eIF2α kinase family: protein kinase R (PKR), PKR-like endoplasmic reticulum kinase (PERK), heme-regulated inhibitor (HRI), and/or general control non-depressible 2 (GCN2). Phosphorylation of eIF2α leads to a decrease in global protein synthesis and the induction of selected genes that together promote cellular recovery, which can cause tumor survival.

As used herein, the term “operably linked” in the context of a nucleic acid refers to a nucleic acid that is placed into a structural or functional relationship with another nucleic acid. For example, one segment of a nucleic acid molecule (for example, a DNA or an RNA) may be operably linked to another segment of a nucleic acid molecule (for example, a DNA or an RNA) if they are positioned relative to one another on the same contiguous nucleic acid molecule and have a structural or functional relationship, such as a promoter or enhancer that is positioned relative to a coding region so as to facilitate transcription of the coding region. In other examples, the operably linked nucleic acids are not contiguous, but are positioned in such a way that they have a functional relationship with each other as nucleic acids or as proteins that are expressed by them. Linking may be accomplished by ligation at convenient restriction sites or by using synthetic oligonucleotide adaptors or linkers. In some embodiments, a nucleic acid molecule described herein is operably linked to a polypeptide. In some embodiments, a nucleic acid molecule including a G-rich 5′ untranslated region (UTR) is operably linked to a heterologous transgene. In other embodiments, a nucleic acid molecule is operably linked to express an antisense RNA.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “subject” and “patient” are used interchangeably and refer to a mammal. Mammals include, but are not limited to, domesticated animals (e.g., cows, sheep, cats, dogs, horses, and rabbits), primates (e.g., humans and non-human primates such as monkeys), and rodents (e.g., mice and rats). In certain embodiments, the subject is a human (e.g., a human having a cancer, such as a human having a leukemia, e.g., a chemoresistant leukemia). A subject to be treated according to the methods described herein may be one who has been diagnosed with a cancer, such as leukemia, a lung cancer, a breast cancer, an ovarian cancer, a head and neck cancer, an esophageal cancer, a cervical cancer, a uterine cancer, a stomach cancer, a bladder cancer, a prostate cancer, a urothelial cancer, a pancreatic cancer, or a brain cancer. Diagnosis may be performed by any method or technique known in the art. One skilled in the art will understand that a subject to be treated according to the present disclosure may have been subjected to standard tests.

According to the methods described herein, a chemotherapeutic and an inhibitor may be co-administered to a subject. Such co-administration typically involves administering a chemotherapeutic and inhibitor together. In some embodiments, co-administration involves administering first a chemotherapeutic followed by administering within, for example, 1 minute, 5 minutes, 10 minutes, 20 minutes, or 30 minutes an inhibitor. In other embodiments, co-administration involves administering first an inhibitor followed by administering within, for example, 1 minute, 5 minutes, 10 minutes, 20 minutes, or 30 minutes, a chemotherapeutic.

Still further, according to the methods described herein, a subject may be administered an inhibitor prior to receiving a chemotherapeutic. In some embodiments, the subject is administered the inhibitor, for example, 45 minutes, 50 minutes, 55 minutes, 1 hour, 2 hours, 3 hours, 4 hours, 5 hours, 6 hours, 7 hours, 8 hours, or even 12 to 24 hours prior to receiving the chemotherapeutic. In other embodiments, the subject is administered the overrider, for example, 45 minutes, 50 minutes, 55 minutes, 1 hour, 2 hours, 3 hours, 4 hours, 5 hours, 6 hours, 7 hours, 8 hours, or even 12 to 24 hours prior to receiving the chemotherapeutic.

In other embodiments, an inhibitor is not administered to a subject who has received a chemotherapeutic. For example, an inhibitor is not administered 45 minutes, 50 minutes, or 1 hour or more after the subject has been administered the chemotherapeutic.

In yet other embodiments, the inhibitors are administered to a subject or a patient according to standard methods known in the art (e.g., orally (e.g., a pill or capsule) or intravenously).

In certain embodiments, chemotherapeutics are administered to a subject or a patient according to standard methods known in the art (e.g., orally (e.g., a pill or capsule) or intravenously).

### BRIEF DESCRIPTION OF THE DRAWINGS

FIG. 1A is an image of a Western blot showing FXR1 protein levels in G0 (2 days of serum-starvation) cells and in AraC (1 μM) treated cells compared to untreated cells. FIGS. 1B and 1C are a series of graphs and images of Western blots showing THP1 survival with AraC chemotherapy by trypan blue cell counting, FIG. 1B (left panel) with FXR1 overexpression (FXR1 OE) compared to vector control, and FIG. 1C (left panel) with FXR1 depletion compared to vector control shRNA cells; Western blot (right panels of FIGS. 1B and 1C) of FXR1 with Tubulin and Actin as loading controls. FIG. 1D (left panel) shows the translation comparison in FXR1 knockdown (FXR1 KD) compared to shRNA control (Control) stable cell lines in untreated (S+) and serum-starved G0 cells by ³⁵S Methionine labeling followed by SDS-PAGE; below Western analysis of FXR1 with Actin as loading control, and (FIG. 1D, right panel) quantification of ³⁵S scintillation counts. FIG. 1E is a set of polysome analyses in FXR1 KD and control G0 cells showing polysomes and ribosome subunits. Data are average of 3 replicates +/− SEM. FIG. 1F shows the comparison of global translation by labeling nascently translated proteins in FXR1 KD compared to shRNA control cells, and in FXR1 OE compared to control vector cells, with I-homopropargylglycine (HPG), an amino acid analog of methionine containing an alkyne moiety that is biotinylated by Click-iT chemistry (Thermo Fisher Scientific), followed by SDS-PAGE and HRP-streptavidin Western analysis; Western analysis of FXR1 levels below and quantification of labeled protein levels on the right. FIG. 1G is a graph showing the fold change in protein levels of selected ribosomal and ribosomal-associated proteins in THP1 G0 cells. FIG. 1H is a graph showing the fold change in protein levels of selected ribosomal and ribosomal-associated proteins in THP1 G0 cells treated with cytarabine (AraC). Data are average of three replicates +/− SEM. Taken together, these data indicate FXR1 is required for translation.

FIGS. 2A and 2B are a set of graphs quantifying qPCR experiments of rRNAs and snoRNAs, normalized to tRNA-met, in FXR1 KD cells (FIGS. 2A and 2B, left panel) or FXR1-OE cells (FIGS. 2A and 2B, right panel) compared to control cells. FIG. 2A shows the fold change of the rRNAs: 45S, 18S, 28S, 5.8S, & 5S RNAs, and FIG. 2B shows the fold change of the snoRNAs that regulate rRNA cleavage and processing, U3 and U8. FIG. 2C is a set of graphs quantifying snoRNA levels from microarray profiles in FXR1 KD THP1 G0 cells compared to control shRNA cells (left panel), and the same in THP1 G0 compared to serum-grown proliferating cells (right panel). FIG. 2D shows the fold change in pseudo uridylation on a rRNA modification site (on 28S at U4966 and U4975) of a snoRA regulated in G0 and by FXR1 from the dataset, SnoRA22 site in THP1 (G0) and THP1 cells treated with AraC as measured by low dNTP CMCT qPCR assay. FIG. 2E shows the results of a 2′-O-methylation assay by low dNTP qPCR of rRNA site (28 S rRNA A4541 m) modified by a snoRD, snoRD63, regulated in G0 and by FXR1 from the dataset, in FXR1 OE cells compared to control cells. FIG. 2F shows Mass Spectrometry data of pseudouridine in rRNA enriched samples. FIGS. 2G and 2H show, by Western blots performed post-co-immunoprecipitation of FXR1 with snoRNA proteins, FXR1 association with ribosome and snoRNA regulators, NOLC1 (FIG. 2G) and DDX21 (FIG. 2H). Co-immunoprecipitation was performed by in vivo formaldehyde crosslinking and FXR1 immunoprecipitation followed by Western blot analysis of FXR1, NOLC1, and DDX21. FIG. 2I is a graph showing partial rescue, by qPCR quantification, of rRNA level of 45S RNA by overexpression of DDX21 in FXR1 KD cells. FIGS. 2J and 2K show Western blots of POLR1 D in FXR1 KD G0 and serum-grown (S+) proliferating cells (FIG. 2J), and in FXR1 OE compared to vector control cells (FIG. 2K). FIG. 2L is a graph showing qPCR analysis for PolR1 D mRNA in FXR1 immunoprecipitated RNA compared to IgG control immunoprecipitated RNA. FIGS. 2M and 2N are a set of graphs quantifying qPCR experiments of rRNAs and snoRNAs, normalized to actin mRNA, in FXR1 KD cells (FIGS. 2M and 2N, left panel) or FXR1-OE cells (FIGS. 2M and 2N, right panel) compared to control cells. FIG. 2M shows the fold change of the rRNAs: 45S, 18S, 28S, 5.8S, & 5S RNAs, and FIG. 2N shows the fold change of the snoRNAs that regulate rRNA cleavage and processing, U3 and U8. FIG. 2O is a graph of snoRNAs from normalized microarray profiles in FXR1 KD G0 cells compared to control cells (red) and G0 cells compared to proliferating cells (blue). FIG. 2P is a Western blot, performed after co-immunoprecipitation, of c-MYC in FXR1-OE versus control cells. Data are average of 3 replicates +/− SEM. Taken together, these data indicate that FXR1 regulates rRNAs and snoRNAs and associates with ribosome and snoRNA regulators.

FIG. 3A is a set of graphs showing RP protein levels by qPCR normalized to tRNA-met in FXR1 KD cells compared to control shRNA cells (left panel), and in FXR1 OE cells compared to control cells (right panel). FIG. 3B is a set of graphs showing expression levels of 18S and 28S rRNAs (representing ribosomes) by qPCR. DEAE fractionation of in vivo crosslinked cell cytoplasmic extracts, followed by ribosome purification with Y10B immunoprecipitation, reveals ribosome migration in G0 and AraC treated cells compared to untreated S+ cells (migrating in distinct salt fractions) as indicated by qPCR analysis of fractions for where 18S and 28S rRNAs co-migrate. FIGS. 3C-3CE show Western blot analysis of RPLP0 (FIG. 3C), RPLP2 (FIG. 3D), other RPs RPL29 and RPL11 (FIG. 3E) in FXR1 OE and control cells with or without AraC treatment. FIG. 3F is a Western blot of phosphorylated PKR (P—PKR) in FXR1 OE cells compared to control cells and with AraC treatment (0 μM, 1 μM, and 5 μM), normalized to total PKR protein levels. FIG. 3G is a Western blot showing that overexpression of snoRD46 (see FIG. 7D for graph of qPCR showing snoRNA amplification), which causes increased rRNA 2′-O-methylation, also leads to increase of eIF2α phosphorylation as shown by Western analysis. FIGS. 3H and 3I are Western blots showing increasing phosphorylation of GCN2 (FIG. 3H) and eIF2α (FIG. 3I) in FXR1 OE cells compared to control cells, normalized to their total protein levels (total GCN1 and total EIF2α, respectively). FIG. 3J is a set of graphs showing fold change in RNA levels, with respect to IgG control, of 28S rRNA (top left panel) and 18S rRNA (top right panel) following immunoprecipitation of GCN2 in FXR1 OE compared to control cells. Protein levels of GCN2 and RPLP0 are shown in the bottom panel. FIG. 3K is a Western blot analysis of eIF2α phosphorylation in FXR1 KD cells showing FXR1 depletion reduces eIF2α phosphorylation. FIGS. 3L and 3M are a pictograph and a set of graphs showing translation ratios of GUG start site Luciferase reporter over AUG reporter normalized to co-transfection Renilla control reporter, as indicated by Luciferase reporter assays (FIG. 3L, left panel) in G0 and AraC treated cells compared to untreated, serum-grown THP1 cells (FIG. 3L), and in FXR1 overexpression cells compared to control cells (FIG. 3M). FIG. 3N is a set of graphs showing fold change of RP RNA levels by qPCR normalized to actin mRNA in FXR1 KD (left panel) and in FXR1-OE (right panel) compared to control cells. FIGS. 3O-3Q are Western blots of RPLP0 (FIG. 3O), RPLP2 (FIG. 3P), and RPL19 and RPL29 (FIG. 3Q) in FXR1-OE and control cells. FIG. 3R is a Western blot of phospho-eIF2α in FXR1 KD versus control cells. FIGS. 3S and 3T are graphs showing translation ratios of GUG start site luciferase over AUG reporter normalized to co-transfection Renilla in G0 or AraC-treated compared to S+ cells (FIG. 3S) and FXR1-OE compared to control cells (FIG. 3T). Data are average of three replicates +/− SEM. Taken together, these data indicate that eIF2α phosphorylation and noncanonical start site translation increase with altered ribosomal components upon FXR1 overexpression.

FIG. 4A is a graph showing polysome analysis in FXR1 OE cells compared to control cells. FIG. 4B is a table showing levels of subunits in FXR1 OE cells compared to control cells, normalized to monosomes, as analyzed by measuring area under the curve. FIG. 4C is a set of graphs showing qPCR analysis of pro-survival gene mRNAs (CD47, left panel; Xbp1, middle panel; Ncam1, right panel) that increase in G0 and AraC resistant cells in polysomes of FXR1 OE cells compared to control cells, normalized for input levels. FIG. 4D is a graph showing gene ontology terms with highest and lowest Normalized Enrichment score (NES) as per GSEA analysis of the FXR1 OE translatome. FIG. 4E is a graph showing alterations in polysome profiles in FXR1 OE compared to control cells by qPCR quantification of mRNA levels on polysomes. Selected mRNAs from FXR1 OE translatome data were amplified by qPCR and normalized to inputs FIG. 4F shows polysome analysis of FXR1 overexpression cells with or without ISRIB treatment. FIG. 4G is a graph of qPCR analysis of pro-survival gene mRNAs on polysomes. FIGS. 4H-4J are graphs showing chemosurvival, quantified as the fraction of cell viability, of FXR1 OE and control cells treated with AraC and ISRIB, which inhibits phosphorylation of EIF2α (FIG. 4H), Toyocamycin, an XBP1 inhibitor (FIG. 4I), and FX1, a BCL6 inhibitor (FIG. 4J), in FXR1 OE compared to control cells and with buffer control. These data suggest that increased non-canonical translation of pro-survival genes in G0 and AraC treated cells, and upon FXR1 overexpression, can be reduced by inhibiting eIF2α phosphorylation, which reduces chemoresistance of FXR1-overexpression cells. FIG. 4K is a graph showing gene ontology (G0) terms with highest and lowest normalized enrichment score (NES), based on GSEA of FXR1 OE translatome compared to control vector translatome. Data are average of three replicates +/− SEM. Taken together, these data indicate that FXR1 overexpression promotes translation of prosurvival genes with distinct 5′-UTRs.

FIG. 5 is a Western blot showing FXR1 protein expression levels in THP1 cells untreated or treated with 1 μM and 5 μM AraC for the indicated amount of time, normalized to actin. The altered forms have been observed in many studies analyzing FXR1 and may include both spliced forms and modifications that cannot be distinguished by available antibodies.

FIG. 6A is a protocol flowchart illustrating enrichment of rRNA from FXR1 knockdown (FXR1 KD) and control THP1 G0 cells for LC MS/MS analysis of RNA modifications (top), followed by a table showing RNA mass spectrometry analysis of the 2′-O-methylated forms of adenosine, cytidine, guanosine, uridine and pseudouridylated form compared to their respective unmodified forms. Experiment was done through Go Beyond RNA arraystar inc., where RNA was hydrolysed to single dephosphorylated nucleosides and deproteinized before mass spectrometry. FIG. 6B is a table showing the top translation factors that interact with FXR1 in G0 THP1 cells. FXR1 was immunoprecipitated from G0 THP1 cells followed by Tandem-Mass-Tag (TMT) mass spectrometric analysis to identify interacting partners. FIG. 6C is a Western blot of FXR1 immunoprecipitation from the cytoplasmic fraction of FXR1 overexpression (FXR1 OE) and control cells (left panel) and graphs showing the corresponding RNA levels of 28S (middle panel) and 5S rRNA (right panel) associated with FXR1 in Control and FXR1 OE cells, normalized against IgG. FIG. 6D is a table showing proteomic analysis (from past TMT mass spectrometry dataset on G0 and AraC compared to untreated THP1 cells) that reveals levels of POLR1 D iso-2 in AraC treated and G0 cells compared to serum grown, untreated THP1 cells. FIG. 6E is a set of graphs showing comparative RNA levels, quantified by ddPCR, of 28S rRNA, FXR1 RNA, and U3 snoRNA in FXR1 KD and OE cells compared to control cells. FIG. 6F is a table generated by TMT spectrometry proteomic analysis, which shows levels of POLR1 D iso-2 upon FXR1 depletion, relative to control, in G0 THP1 cells. POLR1 D iso-2 is a Pol I and Pol III regulator subunit that is increased in G0 and AraC-treated cells compared to untreated THP1 cells. Data are average of three replicates +/− SEM. Taken together, these data indicate that FXR1 expression affects rRNA and snoRNA levels, as well as rRNA modifications, and that FXR1 interacts with ribosome and translation regulators.

FIG. 7A is a set of graphs showing RNA levels via qPCR of 28S and 18S rRNA, which represent ribosomes, following DEAE fractionation of in vivo crosslinked cytoplasmic extracts. Ribosome migration occurs in G0, AraC treated, and FXR1 OE THP1 cells compared to untreated S+ THP1 cells (migrating in increasing salt fractions), normalized to their levels in the inputs. FIG. 7B is a pictographic representation of the experiment done in FIG. 3B. Briefly, mature ribosomes were immunoprecipitated from cytoplasmic fractions of in vivo crosslinked cells, followed by DEAE fractionation using buffers with increasing salt concentrations (FIG. 7B, top left panel). Immunoprecipitation of ribosomes with Y1 OB antibody is verified by association with 5.8S rRNA. Shown qPCR of 5.8S rRNA level after immunoprecipitation with Y1 OB antibody in THP1 S+, FXR1 OE, THP1 G0 and THP1 AraC cells normalized to its level in inputs (FIG. 7B, top right panel). A Western blot performed after Y1 OB immunoprecipitation showed RPLP0 expression, verifying ribosome pull down (FIG. 7C, bottom left). Levels of 45S, 5.8S rRNAs after immunoprecipitation with Y1 OB antibody from FXR1 OE and control cells were quantified by qPCR analysis, verifying pull down of mature ribosome and not pre-ribosomal forms (FIG. 7B, bottom right). FIG. 7C is a table of RPLP0 in FXR1 KD cells normalized to control cells. FIG. 7D is a set of graphs showing the fold increase in SnoRA2A (left panel) and SnoRD46 (right panel) RNA levels, normalized to tRNA-met, in THP1 cells overexpressing SnoRA2A (left panel) and SnoRD46 (right panel). FIG. 7E is a set of graphs showing a ratio of Luciferase RNA levels to Renilla RNA levels, normalized to actin, by qPCR of the GUG reporter and AUG reporter in G0, AraC treated and FXR1 OE cells. FIG. 7F is a table of RPLP0 fold change of total mRNA levels in FXR1 KD G0 cells compared to control cells. FIG. 7G is a pictographic representation of the ribosome immunoprecipitation followed by the DEAE fractionation protocol. FIG. 7H is a set of graphs showing the fold change in 5.8S rRNA levels after immunoprecipitation with Y1 OB antibody in S+ and AraC THP1 cells normalized to input levels. These data confirm that immunoprecipitation of ribosomes with Y1 OB antibody is successful. FIG. 7I is a Western blot of BT549 breast cancer cell lysates with control and FXR1 shRNAs showing levels of eIF2α phosphorylation upon shRNA-mediated FXR1 depletion. FIGS. 7J and 7K are Western blots showing changes in protein levels over a time course of FXR1 and p-eIF2α in control shRNA and FXR1 KD cells, as well as in control vector and FXR1 OE cells, treated with 500 nM (FIG. 7J) and 5 μM AraC (FIG. 7K). Tubulin and Vinculin are loading controls. p-eIF2α is quantified over total eIF2α levels. Some level of loss of doxycycline-induced shRNA depletion of FXR1 occurs over time likely due to stress signal interference from AraC treatment, resulting in variable residual FXR1 detection in these long exposures. FIG. 7L shows the ratio of Luciferase RNA levels to Renilla RNA levels, normalized to actin, by qPCR of the GUG reporter and AUG reporter, in untreated proliferating cells, G0 cells, AraC-treated cells, control vector cells, and FXR1 OE cells, for Luciferase reporter assays in FIG. 3S-3T. Data are average of three replicates +/− SEM.

FIG. 8A shows the secondary structures and corresponding ΔG values of the 5′ UTRs of top translated genes from the upregulated gene set translatome of FXR1 OE cells, as predicted by RNAFold. FIG. 8B shows the enriched motifs present in the 5′-UTRs of the common set of genes that are increased at the protein level in THP1 G0, and AraC-treated cells, and are elevated in polysome association in FXR1 OE cells (generated by MEME analysis). FIG. 8C shows a table of known non-AUG bearing mRNAs that show elevated polysome association in FXR1 OE cells. FIG. 8D shows the enriched motif present in the 5′-UTRs of the genes with increased polysome association in FXR1 OE cells and with known non-AUGs (generated by MEME analysis from the material in FIG. 8C). FIG. 8E shows levels of ATF4 in FXR1 OE cells compared to control vector cells. FIG. 8F is a graph of CITED1 levels from FXR1 OE and KD translatomes compared to control cells. FIG. 8G is a graph showing results from a luciferase assay of BCL6 5′ UTR reporter normalized to Renilla co-transfected control and for reporter RNA levels over that of control 5′ UTR reporter (CX) normalized to Renilla co-transfected control and for reporter RNA levels, in FXR1 OE cells compared to control vector cells. FIG. 8H is a table of time points of harringtonine initiation inhibition assay, from 0 sec to 300 sec for puromycin labeled 37-10 kDa translated proteins, comparing between FXR1 KD and control shRNA cells, as well as between FXR1 OE and control vector cells. Data are average of three replicates +/− SEM.

FIG. 9 is a plasmid map of the BCL6 5′-UTR-Luciferase construct described in FIG. 8G.

### DETAILED DESCRIPTION

Unless defined otherwise, all technical and scientific terms used have the same meaning as commonly understood by one of ordinary skill in the art to which this invention belongs. Conventional methods are used for the procedures described herein, such as those provided in the art, and demonstrated in the Examples and various general references. Unless otherwise stated, nucleic acid sequences described herein are given, when read from left to right, in the 5′ to 3 direction. Nucleic acid sequences may be provided as DNA or as RNA, as specified; disclosure of one necessarily defines the other, as is known to one of ordinary skill n the art.

The term “comprise” is intended to mean “include”. Where a term is provided in the singular, it also contemplates aspects of the invention described by the plural of that term. The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The following disclosure, as is discussed below, provides, inter alia, 5′-UTR molecules and various modifications thereof, as well as cells and compositions including or targeting such molecules. This disclosure also provides various methods of making and using these molecules, cells and compositions. Methods of administering and treating subjects e.g., humans) with 5′ UTRs or with compositions targeting 5′-UTRs.

### 5′-UTRs

In one aspect, the disclosure relates to 5′-UTRs, which are G rich. Such 5′-UTRs have G-rich motifs that cause these regions to form structures which promote inefficient expression and are not favored during canonical translation conditions. However, these structures are selectively amplified under non-canonical translation conditions, such as conditions in which eIF2α is phosphorylated. Exemplary UTRs typically include motifs such as:

[symbol]

[symbol]

[symbol]

[symbol]

[symbol]

Motif 1
(SEQ ID NO: 13)
GCR¹CR²R³CR³R³R³R²R³CR³CR³R²R³CR²CCR³CR²GCR²R³CCR²ACC
CCR³R³GCR¹CGC

wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside; and
each R³, independently, denotes a guanosine or cytidine nucleoside.

[symbol]

[symbol]

[symbol]

[symbol]

Motif 2
(SEQ ID NO: 14)
R¹CR²CR³R²R³GR⁴R²CR²GR⁵R²GCR²GCGGCGGCGR³R²GGCTGR²GG
CGGCGGCGR⁴R³G

wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside;
each R³, independently, denotes a guanosine or cytidine nucleoside;
each R⁴, independently, denotes a cytidine, guanosine, or adenosine nucleoside; and
each R⁵, independently, denotes a cytidine or adenosine nucleoside.

[symbol]

[symbol]

[symbol]

[symbol]

Motif 3
(SEQ ID NO: 15)
GCCR³R²R¹GCCGR³CR⁴CCR³CCGCR¹

wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside;
each R³, independently, denotes a guanosine or cytidine nucleoside; and
each R⁴, independently, denotes a thymidine or guanosine nucleoside.

Such 5′-UTRs are about 10-1500 nucleotides in length and may be naturally- or non-naturally occurring. In embodiments, the 5′-UTR useful in the compositions and methods described herein are synthetic, being produced according to standard methods known in the art such as those described herein.

In embodiments, the 5′-UTR molecule is between 10-1500 (e.g., between 10-1500, between 50-1400, between 100-1800, between 250-1200, between 500-1100, or about 1000) nucleotides in length. In some embodiments, the 5′-UTR molecule is between 50-1400 nucleotides in length. In some embodiments, the 5′-UTR molecule is between 100-1300 nucleotides in length. In some embodiments, the 5′-UTR molecule is between 250-1200 nucleotides n length. In some embodiments, the 5′-UTR molecule is between 500-1100 nucleotides in length. In some embodiments, the 5′-UTR molecule s about 1000 nucleotides in length.

AU-rich elements in the 5′-UTR region are known to affect mRNA stability. In some embodiments, 5′-UTRs include AU-rich elements. In some embodiments, 5′-UTRs do not include AU-rich elements.

In some embodiments, the 5′-UTR molecule may include a chemical modification. In some embodiments, the chemical modification includes a N6-methyladenosine. The 5′-UTR molecule may further include modifications as described herein.

The following tables provide exemplary 5′-UTRs useful in producing the various compositions, cells, and methods described herein.

TABLE 1
Non-canonical/G-rich 5′-UTR sequences

| SEQ ID | Gene | |
|---|---|---|
| NO: | Symbol | 5′-UTR Sequence |
| 16 | BCL6 | acaagcgagctggtggttgaagctggttaaagaacagcctaggtattccagaagtgtttgaggatcccttccatg |
| | | aaggaagagaggaaagtttttaagtaaacctcccactcccatgtgtcttcagctttcttttgcaaaggagaaaatc |
| | | cttgaagtttggtaaagaccgagttagtctatctctctttgcctatctcgagttgggctggggagaggaggagatag |
| | | gttcttttgtctttttctgtcttctcccttccccacttccttccctccagtccccactcactcacatgcacacactaaccttg |
| | | gagccgatgggattgagtgactggcacttgggaccacagagaaatgtcagagtgtttggttacagactcaagg |
| | | aaacctctcattttagagtgctcatttggttttgagcaaaattttggactgtgaagcaaggcattggtgaagacaaa |
| 17 | ERCC6L | atgcgcggggcgggagtgagcgaaattcaagctccaaactctaagctccaagctccaagctccaagctccaa |
| | | gctccaaactcccgccggggtaactggaacccaatccgagggtcatggaggcatcccgaaggtttccggaag |
| | | ccgaggccttgagcccagagcaggctgctcattacctaagggtcttgctgtgtcgcccagactggaattcagtg |
| | | gcctgatcatagttcactgcagcctcgaactcctgggctcaagcagtcctcctgccccagcctccctagtagctg |
| | | ggacttaagatatgtgaaagaggccaaagaagcaactaagaatggagacctggaagaagcatttaaactttt |
| | | caatttggcaaaggacatttttcccaatgaaaaagtgctgagcagaatccaaaaaatacaggaagccttggag |
| | | gagttggcagaacagggagatgatgaatttacagatgtgtgcaactctggcttgctactttatcgagaactgcac |
| | | aaccaactctttgagcaccagaaggaaggcatagctttcctctatagcctgtatagggatggaagaaaaggtg |
| | | gtatattggctgatgat |
| 18 | NEGR1 | gagtgcgagctgaaagctgctggagagtgagcagccctagcaggg |
| 19 | PECAM1 | ggtttccagccatggctgccattacctgaccagcgccacagccggtctctctgcaggcgccgggagaagtgac |
| | | cagagcaatttctgcttttcacagggcgggtttctcaacggtgacttgtgggcagtgccttctgctgagcgagtcat |
| | | ggcccgaaggcagaactaactgtgcctgcagtcttcactctcagg |
| 20 | CCPG1 | gcccagccggtcaccgcctgccggctgcagacgcctgcgagcaggttgtttttataagaggcgtcattggcgcc |
| | | cgagctgtgaccgccgccactggggcagccagcacaatcgggcggaggtggcgctgccccttcagacctga |
| | | aag |
| 21 | LSAMP | gctgtgtggctgtttctgtacactcactggcaggcttggtgccggctccctcgcccgcccgcccgccagcctggg |
| | | aaagtgggttacagagcgaaggagctcagctcagacactggcagaggagcatccagtcacagagagacc |
| | | aaacaagaaccctttcctttggcttcctcttcagctcttccagagggcttgctatttgcactctctcttttgaaattgtgtt |
| | | gcttttacttttcacccttctgcttgggttttatgagggctttgttaagtcttagagggaaaagagactgagcgaggga |
| | | aagagagaggcaaagtggaaaggaccataaactggcaaagcccgctctgcgctcgctgtggatgaaagcc |
| | | ccgtgttggtgaagcctctcctcgcgagcagcgcgcacccctccagagcaccccgcggacccgcacctcggc |
| | | gtggccacc |
| 22 | MSL3 | gcacgcgcggttgggagcctcgcccatgctttgtcgcgttaccggggctaccgtttgcgcccccgactgcaacg |
| | | gtggccgctgagggaggaggcttctcgaatacggtttctgtcttcgcgttaa |
| 23 | SRSF2 | agttgttactcaggtgcgctagcctgcggagcccgtccgtgctgttctgcggcaaggcctttcccagtgtccccac |
| | | gcggaaggcaactgcctgagaggcgcggcgtcgcaccgcccagagctgaggaagccggcgccagttcgc |
| | | ggggctccgggccgccactcagagct |
| 24 | TPM3 | atttcagggcgtagcaggcggaagtaagggtgagaggaggctgcaacgccgagcggaggaggcaggaac |
| | | cggagcgcgagcagtagctgggtgggcacc |
| 25 | TDRKH | gcggggaagtggcggctgaagcgccgccggcggggctcactgtggtggtgtgagtgggaggcggcggcgc |
| | | tggtggctgcagctggggtgaggcgcgaggcggcgcactcgacggctgactggagcagcggatataagtca |
| | | aagagtacagcagaaa |
| 26 | FCGR2A | acagtgctggg |
| 27 | GCLC | aggagcggcgagctagcggacgcaaagactgggcatgctccgcggcggcgcaggttttggtcacaagtagg |
| | | aagaagccagtgcaccagaccggcaaagagaagcgggagccgccgcggcagcgcggccgtggggtcc |
| | | gccgccgccgcatcggagcgggaggaggagcagcggggagggcgaggccgccgggccgagagccgtc |
| | | ccgcctgctctcggtcttctgccttcgcctccgcgcggtgcgtcggacccagggtctgtcacctgggcgccaggg |
| | | gccgccgccggggagccggagcgggcaggaccctccctccgccgactgcggcccgagagcgcccccgcg |
| | | gggtggagcggcagccgccttctgcgggcggctgagtgtccgtctcgcgcccggagcgggcgaccgccgtc |
| | | agcccggaggaggaggaggaggaggagggggcggcc |
| 28 | SIPA1L1 | agtgcgtgcgtgagcgagtgagcgaaaaggagagcgcgcggcggacgcgcccgggacgcgcggcggca |
| | | ccgggaggccgggccgagcggttttgagagattacctgaagttgcactcaaaagcaagatatactactaacttt |
| | | gaaatggaataaggccctccctatcctgaagacattagctgagagtctggcaccttttaaaggtctgatcatcaat |
| | | tgtctctctgggcggccacctatgagacttcatctgtgtgctttataaatagcagtccagggcaacagtacttgata |
| | | aaaaggttataccttattggtgtggacgttgtctaaatttcggtagccatggcacaagaatataagaaagcatgg |
| | | gattatggcaaccacagaatctcagtagtacaagttccattcagttttttctgaaagaaagccctctgttaaagtga |
| | | agcaaagaaactgttgtggattataacgtttagaagttccaatttttcagtgctttacaaataaagcatcatttaacct |
| | | tttaaatgaaaaagattaagatctcatgcaactgttgtattttctggaagccattctccaaaagggaagtgcacattt |
| | | aaaacacagatatgatggtccttgctgcagggatttaagtctacttgcttttacatc |
| 29 | SEMG1 | agacaaggttttccaagcaag |
| 30 | PDK3 | gctgtcctggagctgctgctgctgctgcggcggctgcaccggcggcgccgaggccgagatcgaggccggggt |
| | | gcgcgcttcgcaaacgtgccctatccgtgcggcttggctgcgccagcccttgcggccacccgggcgtctaggc |
| | | gggtctgtgcgccgcccgggcgagg |
| 31 | GOLGA5 | gcttcccttccacgtctcccggaagcggtagcagggccccggcgggtcggggaggaggtttactcagcttggg |
| | | ccccctccgggccagccgccgagggggcgcggcccaggacggcggctaggccgtagtgcagcctctccgg |
| | | agtcctcaggtttgccaataggattatcctgctgccatc |
| 32 | NCOA1 | ggcggcggtggcggccgaggaggagaacatggcggccgcggagagcggctgaaatgcctgttcttcaggc |
| | | cgggcgagcgggagtctgacgcgatttgctgagctctgtcctccgacggctgcctcgcggccttgagggcctcc |
| | | gccgcctgttcgctgctgctccctcgagcggagcctgcgtcaggttccctctgcatccccctgcggggggacccc |
| | | tgctccggaggagggggccggagagccgcggcgccgggcccgaggagcggcggaggccggggcggcg |
| | | ccgccgccacggtcgcggacgagtgcggcgccggtgagcggggcccagaggcggcggcggcagatctat |
| | | ccatactaatggaaatgtaactggaactgactctgatgataaaatcaaggaccatcaagcaagatcatgcagt |
| | | aggcaactttgcttccaaaagaagttaccaacatttagaatttctacttattctgaggttccagttacagctatatca |
| | | gagaatgagttaatctcctcagaaatcactaaatactactctgaggggcttagaaattaacaggttgtttatataat |
| | | tggccttaaatgaggtgagagtgaagagactagagccatctctggaaaacatcattatcccattccccgggaa |
| | | gctaccctctggaactcaagatttgaccatatctgttttgaggattcattatgaacaaagaagtctcccaggtgtga |
| | | agtttttcaac |
| 33 | ATP6V1D | acaaccagctgactcccgtagaggaagacactgtggaggccagttctggagctattgcagcctcggttgcccg |
| | | gccggggacccgagccgaaaagttatcgtcaga |
| 34 | XPOT | actctccaagagactgctggcgccggcgcccgcccgcgcggcaccgacgcggggagcgcgcttcgcgctg |
| | | actcagcggcggcggcggctgcggcggcggcggcggcgttagccggccctcgcgctctttcccttcctggggc |
| | | gccgaccccgcccgcttgcttgcttgcttgcttgcctgcctgcctgcctgcccggcgccacgcaagagaaggtg |
| | | ccaggggacgcagagcgactagaggcgcgcggtcccggccagcaccgtctctggcgttgtagctgcggccg |
| | | tggcggaggactacggcgacaaggacgagggccgctctcccagctctctgcgtgccgcgccgctccgctccg |
| | | ctggctgaccatctggagtgcaggctgggaggcgggatggagtgatagggaagatgtttataaattcttctgtgg |
| | | gatcagagggcacgcctattacaaccagaaaactacaagtataacagcgagg |
| 35 | ANKRD45 | gctatggtaaccgaggaggccggggcccgggtgtggtatttttggagtta |
| 36 | INTS12 | gtttccgaagggaccaccgggaacagacggatcggcagggcggggcggaacggcgtttgca |
| 37 | TRAK2 | ggcagtgggagcagcggcagcagcttcggctgctgctttcaggctgccgctgcattaggggcttcctgaggaa |
| | | gcgcgggcggacgacagaggatgccgaaccactccagtcatgactgtccaaagtatgataatcacatgaga |
| | | gtgctcgttgctacggatgtcatttgactcatcagagaaaatctgtctaaaagaaaatatccatgtgaccaaatcc |
| | | atttcattattgaatggcttgatggatttcctttactctgattcataccaaagctgtccttctcaaccaaagcaagaaa |
| | | ggatcctgc |
| 38 | CHPT1 | gccccacccgcgagccgcagccgcggccccacagcttctggggctggggccccggcagccgggcaggcc |
| | | ggcctgacctcgacctccgccgtgcgggcccgaccggtgagtccagcccggcagtcgcaggacccggccg |
| | | ccagcctctccctccacctctccctgcccccagcgccaggcgcgggctgcgctcggtggcggcggcggggcc |
| | | ctcaggcggcc |
| 39 | SLCO4C1 | agtcgggagaacccatccagac |
| 40 | ERLEC1 | ggaggcggcgttgccgggctctccggaaggagacgtggcggcggttgggccggtgatacccgggcgctttat |
| | | agtcccgccgcctcctcctccacctcctcctcctcctcctctcctcctggagcagaggaggttgtggcggtggctg |
| | | gagaaagcggcggcggagg |
| 41 | CD47 | gcagcctgggcagtgggtcctgcctgtgacgcgcggcggcggtcggtcctgcctgtaacggcggcggcggct |
| | | gctgctccggacacctgcggcggcggcggcgaccccgcggcgggcgcggag |
| 42 | CDC42BPB | gggctcgggcgccggccgggcttgcgcagcgctaggctcctcgccgctcctagtctgcggcggcggcggcgg |
| | | ggagggcgcggccgcgggcggggctgagggcggcgggggcgggccgcccgagctgggagggcggcgg |
| | | cgccgaggggaggagagcggcccatggacccgcggggcccggcgccccagactctgcgccgtcgggac |
| | | ggagcccaagatgtcggcctaggccggggcgcgacgacgcggacggggcggcgaggaggcgccgctgc |
| | | tgccggggctcgcagccgccgagcccccgagggcgcgccctgacggactggccgagccggcggtgagag |
| | | gccggcgcgtcgggagcgggccgcgcggcacc |
| 43 | CYTH3 | ccgggccgcgcgctgaggagccgcccggtcgcctgcgcgctccctccggcggcgtccccagcccgcggcc |
| | | cctctgctgccggcccccggctcgccggctgcgggagtggcctcaagatggatgaagacggcggcggcgag |
| | | ggtggtggcgtgcctgaagacctctcattagaagagagagaagaacttctagacattcgtcgaagaaaaaag |
| | | gaacttattgatgacattgagaggctgaaatatgaaattgcagaggtgatgacagagatcgacaatctaacttc |
| | | cgtagaggagagcaaaacgactcagaggaacaaacagatagccatgggaagaaagaaattcaacatgg |
| | | atcccaaaaagggaattcagtttctaatagaaaatgacctgctacagagttccccagaagacgtcgcccagttc |
| | | ctttataaaggagaaggcctaaataagaccgtcattggggactacctgggtgaaagggatgaatttaatattaa |
| | | agttcttcaagcctttgttgaactccatgagtttgctgatctcaaccttgtacaagccttaaggtaagtatgatcatttg |
| | | ctgggtgtagagtggatggggaaggccagaggaccactgcctgggcagcagtggcggtgaattgactccag |
| | | gtaacttggtgccacacgagtctcacagatgctggctccttttccggatgatccagctaatgctgtgtgacactga |
| | | gcagccctagacaggaattggggcacacgcttaagataagggccgtgctttcctcactgtggccgctgatgctc |
| | | cttccattccgtttccttttatttgtaatggtgtatggcagtagtcgagtgccttgttcccaagctctgtatggcgtggcgt |
| | | tggtgctccatggggttacttccttccacacttgacccaaagcaccaaacgcatggatacctgagtgtgccagag |
| | | aggatgtaggtgtctcgccattttggtgtctgggacacactccctggatgcccccttgagctgcccttcctgcacgtt |
| | | gtcttcctcctgctctgcaagcaggaagttgtctcgcttccttccttggtgccctggctgagtcgcgtgggctcttcat |
| | | ctgttctgggccttgaccctgagccagcactcagcagcatccgggggagaggaggcagggcttctggtggga |
| | | gtcgcctcagccggcccttggactaagaaaatcaggccgcacctcgtcatcttctcaaatcttcggcttcatacctt |
| | | gttctcgtttacgtggactgtttgctc |
| 44 | SAV1 | agtggtagcggttattcggcggcccgcggcggaccatggccctggccctggcccggcgtcgctgggctttcctc |
| | | acggcgtccccgagcagcgtcgcagagcgggccgacttccgggaaggaactgaccagcgactgagcggc |
| | | ggccggcgcgcttagcgccctgaacatgcggcagtccctgcgggcgaccccgggctccggacaggcggcg |
| | | gcggaggcggcggctcgggagggaaggaggcggcggcgccggcggaggtggcggcggagacggccg |
| | | gcgcccggcgcggagccctagggaggcagttcagcgcggcctcgggcctcgtcgagaagg |

TABLE 2
Non-canonical/G-rich 5′-UTR sequences

| SEQ ID | Gene | |
|---|---|---|
| NO: | Symbol | 5′-UTR Sequence |
| 45 | FUNDC2 | gacaccgcac gctgagctct gtgatgtagc cgcttgcgga gactgcaagc agccgcggcg |
| | | cgcccggccc tccctcttcc gctgccgccg tggga |
| 46 | GSPT2 | atatctgctg ctgccgccgc agttgcgaat gcagcatcgg cgcttagctg cctccgcggt |
| | | gcagctaagg ttcgtgtcgc taccccttgg cccttcgctc ttgctgcctt aaccccgccg |
| | | gcggagcccg ctcttctggc ctgttgagcc cgctccctca ctgccacaca gcaagttccg |
| | | agacc |
| 47 | ZNF600 | agtcctgggc gcgcacaaac ccggaagcgg atcgcgtgga gtgaaggtcc taccacggcg |
| | | cgtgagtttt gctctgcctt ggattaagtc tgcacttccc aggtccccgg cgcttctgcc |
| | | cctgggacgt gggatcccca cggacctgga aattctcgcc tgtcttccct tcacccagag |
| | | caaattgaga cgtcccggag gaagaccaag gcagcctatt gggccttcca ggcaatcaca |
| | | tgggaatcag ccacacgtcc ttcctcctca cctcagaaca tctcagaata acttggtgaa |
| | | atgtctccca ctgtgagcct cagtgagccc acctgtaaca tagaggcctc gcccctgagc |
| | | tctacaatcc tgtgtccagt tgtctcctca gctgtctcct gggtcatcaa acaggcatcc |
| | | ccaccttcag gtgtccacga gtggctttct aaacccccaa acacatttcc ttgcagtctg |
| | | cacatctcag atgagggtga ctacgtactt ccggaaacgg ccgaacttga cagcatgtat |
| | | tttaaatttg tgaaataaat tactttattt gtaagtgttg taatttataa tataaagaga |
| | | aacttagatg tatacgtgaa aagagtgaga agatacatca cttccaattt ttgtttgttt |
| | | gtttgttttt ttgagaggaa ttttcactct tgtggctgag gctggagtgc aatgccatga |
| | | tatcagctca ctgcaacctc tgactcctgg gatcaaggga ttctccttcc tcagactccc |
| | | gagtagctgg gatcacagtc gactttcaaa attctttaag gattgattcc taaagactc |
| 48 | ZNF14 | agtgcggacg tcgccattcc tggcccatgg gaagattgcg tttcacctgc tcctgaaggc |
| | | cgaaggtggc tctagcgcat cctttgtcgc gccgtgacct gcaggtactg acagatccgt |
| | | agggaggaca ccgtgacttc ccggacgctg ggaa |
| 49 | RRP7A | gggtggcaag |
| 50 | RBMX2 | ctttcccggg cgctgattcc tgagtgctga gcgcgaaccc gaggag |
| 51 | ARMCX5 | ctcccactgc cgttgtgggt aacgcggacg tggaagaacc tcgtctgcgg aggaaaaggt |
| | | agatgttaaa tggtaactac gcgcgaggtt ctgaggagcc ctgggaacag gaaggagaaa |
| | | agaataccaa aagtgacaac agtttgccaa tcgcagtctt taatctgata aagcggttat |
| | | ctcgtcttga gtcccaggtg ccgagtcaat ccccatacac agccgccgcc attgcctcga |
| | | gtccttgtgt ctgactgtct gttcctgctg ctgtatgaca cagcacctcg aggcaaggaa |
| | | ataagaaaac tgcctctgat ccaagcagag aaggtagatc tgtccccagg tctgtggaac |
| | | tgtcagttgt gaagttttgt aaaatggtca cccaacttaa aactaggaaa ttacgaagaa |
| | | gagaaaattg ccctgtatct gttaaggtct gcctgtagat ctgctgtagg gcttgtcacc |
| | | attggaagca aggtcctact tcagtggcag atctggtggc cttggagtgg ctgaagacca |
| | | ccaccctcca cagggctggg cccatgcaca gccatccttc cctaccttga gtgagcttcc |
| | | tctgcatgtt ttctatatca ctggcagagc ctgtagttgg aaaggggaca gagtgactac |
| | | tggactttgt gtgaaaacac caaccgggac aaaacttcag tcaaggctga gacgggtggg |
| | | ggtatataac ttgtccttac gttaaacttg gaac |
| 52 | RSL24D1 | acttcctctc aagcttggcg tttgtttggt ggggttacac gcgggttcaa c |
| 53 | TAF6L | agagggaatg agtgtgagct cgtgagtggg cgccgccgcc accgcccccg ccgccgtcgt |
| | | ctcggtagca gccttcgcca cgccggggtc ttcagctcca ctggggcc |
| 54 | KCTD10 | agtccgggtt tggagactcc tgcgtcctcc gacttttc |
| 55 | DDX39B | agatttccct ccttcgtcgc tgttgctgcc gccatacgcg ctctccctgt ttagctcttc |
| | | tgttagaaat agtatctttg ttttcctttg ctgttcctca atcccctact cttcacccct |
| | | tgttttcacc tattttgcga gaacccatcc agatccccct tcccttcttc ccctgccggc |
| | | ccagtt |
| 56 | TTC33 | gatttgtggt cccggagaag agattggcgg aggggaaaga ctgagtcgtc tttcctttgc |
| | | ccgcttctgt ggactgttcg aacgcccagg ggtgggccaa acccgtttct taggaaacca |
| | | ggaatccagg gaaggcaga |
| 57 | DHPS | agttacatgc tggtacggta acctgaggag gcgaggactg attcttggtg tgggggcggg |
| | | ttctaggtac atttaaagct ttctggaatg ggcggagcct ggggcaagac aaattaaggg |
| | | aggatatggg aggaggagcc taagtctggg cggttcttga atttagattt gcttttccca |
| | | gcggggaagg gaccggatct gaaaggagat gctctctgat tcctaaaagg gtgggggctg |
| | | gctgggcgcg gtggcgc |
| 58 | ZNF710 | cctctgcgag ctggcttagg aggagctgtt ttgcatccct tcacgtcagc cctgcctcat |
| | | tcccttcttc cagggaggga gagagcgcga gcgagagagc gagcgagagg aggggggaga |
| | | gagagaaaga gaggagccgg agggagggcg gcaggagcag gcggcgggcg cgcgtggagg |
| | | cgggcggcgg gcgcacagca gcagcccggg cggtgggcag ccaggagccc ccggcccggc |
| | | ccggcccggc ccgccgaggg ccccagcgca ggagccgcgc ccggacccag gcgatgccct |
| | | cctagctagc cgtcacggg |
| 59 | GTPBP3 | ctgcccagac ttgaagccac acaggcaggt cgggcaggcg ggtcgcaggt tgtaaatcc |
| 60 | ZNF408 | aggctttctg acccggaatg gaggaggcgg aggagctgct cttggagggg aagaaggcgc |
| | | tgcaactcgg tgagtgacct gcg |
| 61 | NSA2 | actctttcct gtcccggcct gcgtggtgtg ggcttgtggg tctttgagac ccgaaaattg |
| | | agagcgtttt cgcactccag cggctgctcc tggcggctct gcggccgtca cc |
| 62 | PLCG1 | gtctgccgcc tcagcctcag ccccaacctc agccgccgcc gttgcgcttg ctcccgggcg |
| | | gtcctggcct gtgccgccgc cgcccccagc gtcggagcc |
| 63 | GCDH | agttgcactg tagcctcggc agtgaaccgg gaggtactac caggtcgccg tcgttgctcc |
| | | gctcgctctg agagagc |
| 64 | RPL5 | ccttttccca ccccctagcg ccgctgggcc tgcaggtctc tgtcgagcag cggacgccgg |
| | | tctctgttcc gcagg |
| 65 | PCBP2 | cccagaccag cagaggcagc agccggagca gccgcagcct gcgccctctc ccgcccgccc |
| | | gccctccgcc cgcccgcccg ccctccgccg ccctccaccc gccccggggt ctctttcccc |
| | | cttcctcctc ctcctcctcc accccccctt cctcctccgc ccgcccgcgg ggcccccctc |
| | | gccttcccgc ccgcccctat tgttccgccc ccggcctccc gcccttcccc ttcccgcccg |
| | | ctcccctttt cccctcagtc gcctcgcgcc tgcagttttt ggctttcacc cccaaccagt |
| | | gaccaaagac ttgaccactc aaagtccagc tccccagaac actgctcgac |
| 66 | RPUSD4 | cttccttgta ag |
| 67 | SAAL1 | agcttccggc acggccttca agcgcgggac gcgacaaagt c |
| 68 | NUDT1 | agaggccacg cccccggaag cggcggtgca ggtacgaaaa gcgcgcgcgg ggattccagg |
| | | agtcgtttct tgccttgatg tactggagca atcagatcac acggcggctt ggagaaaccc |
| | | agggacc |
| 69 | ACOT11 | atttggagtc aggcctggct gttgctcagg tgaccagctt gtgtctctgg gagggcgctg |
| | | ctttccccgg ccacccggcg cg |
| 70 | AAMP | gactctggga ggcgtttggg cccagagaag tggatccgcc gcttgcgccg c |
| 71 | URGCP | agcggagacc tcgctgcgct c |
| 72 | CORO1B | agactcctgc ggatgccatt atgaggagca agaacgcgaa ggggcctcgg gtccgctagg |
| | | ccgggatccg gagccgcccg aagccggtgc cgcagccccc tgcgcccccg gtgcccccga |
| | | c |
| 73 | UCKL1 | gggtgatcct cttatgcctg tggccttggc ctctgaggac ggcagtgagt gctgttccct |
| | | cagtacggtg gaggcttcaa gggcctccta gctgctgtgc ctctgctgac atttggcagg |
| | | cagcttctgc cagccaaggg tctcacccca gccacccggc tctgcaccca ctgtgctgcc |
| | | cacaggagtg gtcctgccca tggaagggcc agttcaggtg gccggagctc ctgagctgcc |
| | | ctaggggact gctgtgggtc tgaggtggtg atgtccccca cggctgcctg cgcctgagcc |
| | | cccacgcatc cacccctggg gccactctgc tgttcaggag cacccacccg tgtcctcgac |
| | | c |
| 74 | ELP2 | gcgtctcttg tttgtgcggc tgaccagttg gcgac |
| 75 | NUDT6 | aacggacgaa ttaagcggcg tggag |
| 76 | TRMT61B | actcgtctgc gacggcgcct tcgcgaaaca ct |
| 77 | RAD1 | acttcctccg cggttcctcg gagccgcctc gctcctcttc agggactttg ctgagaaggg |
| | | ctctcgggcg tccagacccc accgcaaagg tgtttggcga tccgccgaga agttgttggc |
| | | cccaggagca tccctcgggg ccgaatgcgc agtggacg |
| 78 | CCDC61 | agttgaaccg ctcgcgagga gggttgctag tggagaagca accttggcc |
| 79 | ILF2 | ctcttcagtt gtctgctact cagaggaagg ggcggttggt gcggcctcca ttgttcgtgt |
| | | tttaaggcgc catgaggggt gacagaggcc gtggtcgtgg tgggcgcttt ggttccagag |
| | | gaggcccagg aggagggttc aggccctttg taccacatat cccatttgac ttctatttgt |
| | | tgtttacttc tggtttagtg tgaa |
| 80 | NR1H2 | gtgacgcaag ggcggggttg ccggaagaag tggcgaagtt acttttgagg gtatttgagt |
| | | agcggcggtg tgtcaggggc taaagaggag gacgaagaaa agcagagcaa gggaacccag |
| | | ggcaacagga gtagttcact ccgcgagagg ccgtccacga gacccccgcg cgcagccatg |
| | | agccccgccc cccgctgttg cttggagagg ggcgggacct ggagagaggc tgctccgtga |
| | | ccccacc |
| 81 | PUF60 | gggcgcggcg tgtggccctt gcgggcgagc ctggcggggt tgcgttcctg tccgctagca |
| | | ggtcaatggc cagcaaggag gggggtccga gccggcggcg gcggcggcag tggtggcagc |
| | | gggagacaaa tggaaacctc cacagggcac agactccatc aag |

In embodiments, 5′-UTR include those having a certain percent identity (e.g., 70%, 75%, 80%, 85%, 90% or even 91%, 92%, 93%, 94%, 95%, 96%, 97%, 98%, 99%, or 100% sequence identity) to any of the aforementioned 5′-UTRs described herein (e.g., the tables above listing various human 5′-UTRs).

As used herein, the term “percent identity” or “sequence identity” refers to percent (%) sequence identity with respect to a reference polynucleotide sequence following alignment by standard techniques. Alignment for purposes of determining percent nucleic acid sequence identity can be achieved in various ways that are within the capabilities of one of Skill in the art, for example, using publicly available computer software such as BLAST, BLAST-2, PSI-BLAST, or Megalign software. In some embodiments, the software is MUSCLE (Edgar, Nucleic Acids Res., 82(5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Aitschui et al. (1990) J. Mol, Biol., 215:408-410). As an illustration, the percent sequence identity of a given nucleic acid sequence, A, to, with, or against a given nucleic acid sequence, B, (which can alternatively be phrased as a given nucleic acid sequence, A that has a certain percent sequence identity to, with, or against a given nucleic acid sequence, B) is calculated as follows:

100 multiplied by (the fraction X/Y)

where X is the number of nucleotides scored as identical matches by a sequence alignment program (e.g., BLAST) in that programs alignment of A and B, and where Y is the total number of nucleotides in R.

In yet other embodiments, the 5′-UTR is a heterologous nucleic acid molecule. Such a heterologous nucleic acid molecule or sequence is a nucleic acid molecule or sequence that (a) is not native to a cell in which it is introduced or (b) has been altered or mutated by the hand of man relative to its native state, or (c) has altered expression as compared to its native expression levels under similar conditions.

The 5′-UTRs may be synthetically generated. The invention therefore also encompasses production of and use of nucleic acid molecules (e.g., 5-UTRs described herein as well as the other molecules herein) which are generated by synthetic chemistry. After production, the synthetic sequence may be inserted, if desired, into any of the many available expression vectors and cell systems using reagents well known in the art. Moreover, if desired, synthetic chemistry may be used to introduce changes into a sequence as desired.

### Start Sites

Constructs typically include a non-canonical start site. Such a start site includes a poor Kozak consensus sequence, such as sequences deviated from RCCRCCAUGG (SEQ ID NO: 98, where R is any purine), or a non-AUG start site. Exemplary start sites are AUA, CUG, GUG, CUG, ACG, or AUC. The following table lists start sites useful in the invention.

TABLE 3
Genes with non-AUG start sites

| SEQ ID | Gene | Start | |
|---|---|---|---|
| NO: | Symbol | Site | 5′-UTR Sequence |
| 82 | MYC | CUG | ggagtttattcataacgcgctctccaagtatacgtggcaatgcgttgctgggttattttaatcattctaggcat |
| | | | cgttttcctccttatgcctctatcattcctccctatctacactaacatcccacgctctgaacgcgcgcccatta |
| | | | atacccttctttcctccactctccctgggactcttgatcaaagcgcggccctttccccagccttagcgaggc |
| | | | gccctgcagcctggtacgcgcgtggcgtggcggtgggcgcgcagtgcgttctcggtgtggagggcagc |
| | | | tgttccgcctgcgatgatttatactcacaggacaaggatgcggtttgtcaaacagtactgctacggagga |
| | | | gcagcagagaaagggagagggtttgagagggagcaaaagaaaatggtaggcgcgcgtagttaattc |
| | | | atgcggctctcttactctgtttacatcctagagctagagtgctcggctgcccggctgagtctcctccccacct |
| | | | tccccaccctccccaccctccccataagcgcccctcccgggttcccaaagcagagggcgtgggggaa |
| | | | aagaaaaaagatcctctctcgctaatctccgcccaccggccctttataatgcgagggtctggacggctg |
| | | | aggacccccgagctgtgctgctcgcggccgccaccgccgggccccggccgtccctggctcccctcctg |
| | | | cctcgagaagggcagggcttctcagaggcttggcgggaaaaagaacggagggagggatcgcgctg |
| | | | agtataaaagccggttttcggggctttatctaactcgctgtagtaattccagcgagaggcagagggagcg |
| | | | agcgggcggccggctagggtggaagagccgggcgagcagagctgcgctgcgggcgtcctgggaa |
| | | | gggagatccggagcgaatagggggcttcgcctctggcccagccctcccgctgatcccccagccagcg |
| | | | gtccgcaacccttgccgcatccacgaaactttgcccatagcagcgggcgggcactttgcactggaactt |
| | | | acaacacccgagcaaggacgcgactctcccgacgcggggaggctattctgcccatttggggacacttc |
| | | | cccgccgctgccaggacccgcttctctgaaaggctctccttgcagctgcttagacg |
| 83 | TEAD3 | AUA | aacacaaactttccgtcccgctcgctccctcctccgcgctcggcgcctcccgctccagcccggctcattcc |
| | | | gcacattccggccagccccctccccacgaccccccttccccggccccccttgcggctccctcgggcccg |
| | | | gcggagcggcccggccggagcgcccccgcgagctcggaccaggctcagccgcccagtgggctcag |
| | | | gcccagagcccagagcaaccagcaca |
| 84 | CITED1 | CUG | gagtccctgcttcgcagagcaccggacttggagtcagagagacctggctgcgaatcccggctccgcca |
| | | | ctttctagcgctgtgaccttggttttcaggtggcgaaactgactggtctggttagagcgcgtccaagccaag |
| | | | ccgcagtgctgtttggatttttaaatttctccttctgagtgagaaaatagcaatcgag |
| 85 | STARD10 | GUG | agtttgccatgatggatgccagaacctgcacgtggggcttggtgacgcgatgactgagatgctggagtc |
| | | | cccggtcagaaggacgatgccgagcgcgccgtattttctcctgcctctcctcagatcgccgccgttgttgg |
| | | | gctgtggagacgtctcaaccaaagtggagccgacagaggccccgggagccgtggtgacagagcgg |
| | | | ccacaaccactagggggcgggaggcagagcggcccctagtccgcccctagtccgggacctctgggc |
| | | | ggtcccgggcgggaagcgcacgcgcaaacgcggggcttcgcggccgctgtaaacaccaaagagc |
| | | | gcctgcgcaagccgagagtctcgggatcgacacgtgggggcgcctgagcgaagataaccgtaataa |
| | | | atagtaacctaacggtccagtcatcgttctgtggtcctttcttttatgattcacaaggaatgaccctcttcatcg |
| | | | cctctcctaattcagtcctcacaacagtccttttacaaatgggacaacaggttagaggaagtcaggcaga |
| | | | tttccagcatcatagagagtaaaggaccagggaaggatcaggattcaaggactgcacccaggctctg |
| | | | cttccagcttgctgtgtgactttgggtaattttgttcccttagggaactgagctttctcatttgtaaatgcaaaca |
| | | | ggctgttgggaggatcaaatgagatccaggggtgaaaacagcttagtttactttcaggaatttacccacg |
| | | | cggtatataaaggcaaaatattattatagtcaggtgattgtagattgaggaacccatttcctcattctgcaa |
| | | | attgcaaacctgagggcccaaagagggacaggggcttgccccaggtctcagcaggctgtgagcaag |
| | | | agctaaagcctaatcctcctgcctttgggcctggagcccttccttgtaccccaggggtcagtgtctttgttgg |
| | | | atacaggcttagattgactgactgtaccctgagaacctaggggagtccctgttcccaattcttctcctaccc |
| | | | ccaccttggcctgatggaggaagaccctgctgtgttgagatgagcaccagagccaagaagctgagga |
| | | | ggatctggagaattctggaggaagaggagagtgttgctggagctgtacagaccctgcttctcaggtccc |
| | | | aggaaggtggcgtcagcatctgcagccgcgtcgacgttgtcggagcctccgcggaggacccaggag |
| | | | agccggactaggaccagggccctgggcctccccacactcccc |
| 86 | GTF3A | CUG | aagtgtgccggcgtcgcgcgaaggttcagcagggagccgtgggccgggcgcgccggttcccggcac |
| | | | gtgtctcggcacgtggcagcgcgcctggccctgggcttggaggcgccggcgcc |
| 87 | VEGFA | CUG | gcggaggcttggggcagccgggtagctcggaggtcgtggcgctgggggctagcaccagcgctctgtc |
| | | | gggaggcgcagcggttaggtggaccggtcagcggactcaccggccagggcgctcggtgctggaattt |
| | | | gatattcattgatccgggttttatccctcttcttttttcttaaacatttttttttaaaactgtattgtttctcgtttta |
| | | | atttatttttgcttgccattccccacttgaatcgggccgacggcttggggagattgctctacttccccaaatcactgtg |
| | | | gattttggaaaccagcagaaagaggaaagaggtagcaagagctccagagagaagtcgaggaaga |
| | | | gagagacggggtcagagagagcgcgcgggcgtgcgagcagcgaaagcgacaggggcaaagtga |
| | | | gtgacctgcttttgggggtgaccgccggagcgcggcgtgagccctcccccttgggatcccgcagctgac |
| | | | cagtcgcg |
| 88 | EIF4G2 | GUG | gccagcagtgagtcggagctctatggaggtggcagcgggtaccgagtggcggctgcagcagcgactc |
| | | | ctctgagctgagtttgaggccgtccccgactccttcctcccccttccctcccccttttttttgttttccgttccccttt |
| | | | cccctcccttccctatccccgacgaccggatcctgaggaggcagctgcggtggcagctgctgagttctcg |
| | | | gtgaaggtatttcatttctcctgtcccctcccctccccaccccatctattaatattattcttttgaagattcttcgtt |
| | | | gtcaagccgccaaa |
| 89 | NPW | CUG | ggctcgcctccagcctcctgcgctccggtacctgggcgtcccaactccactgcgcgcccaaacccagc |
| | | | cgagccggttcgtggcccgccccgccgggcggccgtcgacgcgagcgcc |
| 90 | NFKBID | CUG | cttaacccttcctccgccggaacccaggctcggaaggtgagcaggggtgagcgcagccactgcccaa |
| | | | cgcaaaccgtgaagaagcttctggaagagcagaggcgccgccagcagcagcagcccgacgctgg |
| | | | cggggtgcagggacaatttctccctcccccagagcagcccctgaccccatctgtgaatgaggctgtgac |
| | | | tgcacactcggagactgtgggttctggacctagcagcctgggctttccagactgggaccccaacacgca |
| | | | tgctgcctacactgacagcccctactcttgccctgcttctgctgccgaaaatttcctgcctcctgacttctacc |
| | | | caccctcggacccagggcagccgtgcccatttccccagggc |
| 91 | WDR26 | ACG | attaggcagcagctgggggaagaatcaacacaccagggagcagagcggagcggatccgaggaca |
| | | | gaggcggcagctgcctccgctgccgcccgccgccgccgccgccgcagccactgccggggctcagcc |
| | | | cagcggcgggccccgcgcccccgcagccgccccgcggcgccccccggcgcgacccccgggccgc |
| | | | gcaccccgagctgcctccgggaggggggggggaaagggcccggatcctccttctccccctcctcctcc |
| | | | tccccctcctctcaccctcaccctcaccctcctaccctgaccccctccccgcctccccggcccccctcacc |
| | | | ccgaccctctccccggcccccgcctcccctcccccgcctcgccccaccggcttcccaccacggcctctct |
| | | | cggcgaggaaactctggcctccgcttcctcctcctccgactcggacaccggcggagcctccccgcccc |
| | | | cgcggaagaaaccccgagcctcggcggcggagggagtaggagagcccggggcttcggcaggcag |
| | | | agcaggcctctcccctccgtcctcgtcgtcctcgtcctcctcctcctcctcctcctccgtggtggtagtggtgg |
| | | | gacttcccccggctgctgcccctcccgccgccgctgctgtcccccaccgaagtagcggccacagcctg |
| | | | gtcagcggcagcatc |
| 92 | RNF187 | CUG | gttggcgtcttcgtcctgttgctggtctccgtccggtcgccggccgtctaggtctccggccctccccagccg |
| | | | ctcctgcgcccttgccggccccgccgcccgcagcc |
| 93 | EIF4G3 | AUC | agccgtcctgccggactgctggaggcggccacagcgccatgttggatgctctgctcgttgagtgaagaa |
| | | | aatccaccggcatcgcctgagccccgctaccgagaagggcgccgcttcctccggggagggggataa |
| | | | agatcccccgccgccggcccatgaggatattgccgtgaaaggcacagcgactgcagcaggaaccgg |
| | | | acccggcaccggagcggcggcggcggcggcagcagcggtaccgcctcctcacccggcggcggca |
| | | | gcagcggcggcggcggcggcggcggcggcggcggcggcagcggtcccccctcctcacccgaacat |
| | | | cagggccctccagactcaggcgccccaacaaattcctagaggacctgtgcaacaacctcttgaggatc |
| | | | gaatcttcactcccgctgtctcagcagtctacagcacggtaacacaagtggcaagacagccgggaacc |
| | | | cctaccccatccccttattcagcacatgaaataaacaaggggcatccaaatcttgcggcaacgccccc |
| | | | gggacatgcatcgtcccctggactctctcaaaccccttatccctctggacagaatgcaggtccaaccac |
| | | | gctggtataccctcaaacccctcagaca |
| 94 | YPEL2 | ACG | agcccccggcccggacccggagggatgcggagtggcggccgcggcggtggcggagactgtggcttt |
| | | | aagagcgtgccgggagcccgagccccagccgggccgcgcttcgccgctgcgcaccccagcggagc |
| | | | caagccccacgctggccggacagggccgcctgtcgccgggctgctgagaactagccctagacctctg |
| | | | cgtgagggttcttctgccgaagacatcaccagtgtgtggagcctgccacacccacccgctgccaaacc |
| | | | acggcctttacctgtgtcttccggtgtttcccgtgcgacccatcctgtgggagtgcctcgtgggctgcccca |
| | | | gagttcaccccacactcagcagcacca |
| 95 | TLE3 | CUG | agtccctatttggaactgctctcgcggcagttcagacctcgtgctcgtccccttcgcctgtctgtgtgtggtat |
| | | | ccgtaggtccggggcacttttttttttggtgtgtgtgtgtgtgcgtgcgcgcgtgtgtgtgtgttgggtgtgtgtgt |
| | | | tttgggggtttgccggggcgcggagaggagagccgggccgaggggaaggaaggaagccgtagag |
| | | | ctccccggtctcgggctgcgtctcccctccagcgctctcctgcctgtgcgctccgacgcggcagcggcgg |
| | | | tggcgctgagcgctcctcggtggggacgcggtgctcaagctgggagcagcgagaaccctttgcggag |
| | | | gcttcccgtgcccgctggacttaggatgccagggcgtttgatttgcacctactgagcccggagttcctccc |
| | | | cggccctgggaagggcttgcagcggcggcggcggcggcggcagcggcagtagcagacggaacag |
| | | | caggctctcctctccgagggggggcgtcgaagcgcccgcgccgggctcccgccctcaccctccggcg |
| | | | ctcctgcggccccaagccaccacctcgtagcccgcctccttcctcttctcgccgcgagccccaggcccg |
| | | | gacgcctctaggatcggacccgcgcgccccggatgcttggggctgccccggcgcgcccccctacagg |
| | | | cgcgacccgctgagtgacccggagggggcccgagctcgccccaggaggggcccccggcctcggg |
| | | | accccgcgccggacctgctggggagccgacgatgtccgcgcagtcccaggcattgacgcgctcgac |
| | | | gcagcccagggaaggcggccggccgctgcccgccgagccctaggcgcacaaagcccgcgcccgc |
| | | | cgcccggccccggcgcccgccgacgactttgccgcctgctccgcggctctttgtctccacttggggcgg |
| | | | gcgcccgactctgggatttcgctgcgagaacgagctgggggggccgggggcgagctctcggtttcccg |
| | | | gccgccccccgctcgggctcggctcccccctcccccgcacctccccggcccgggctctcggcgcttcca |
| | | | cgctctcggaatcacgacccctccctgcc |
| 96 | ZFP62 | GUG | gtgcggtccgcgcttttgtcccgctggcggccgggttcccgcggcgccgccacagcc |
| 97 | EPHX3 | ACG | actccaggtgtcgcccacttaactgaagatccaggatcccctccttaaggtgcccctggccggcagcgc |
| | | | cgccgtggtcccggagcgcggcgac |

Such start sites described above may be synthetically generated.

### Expression

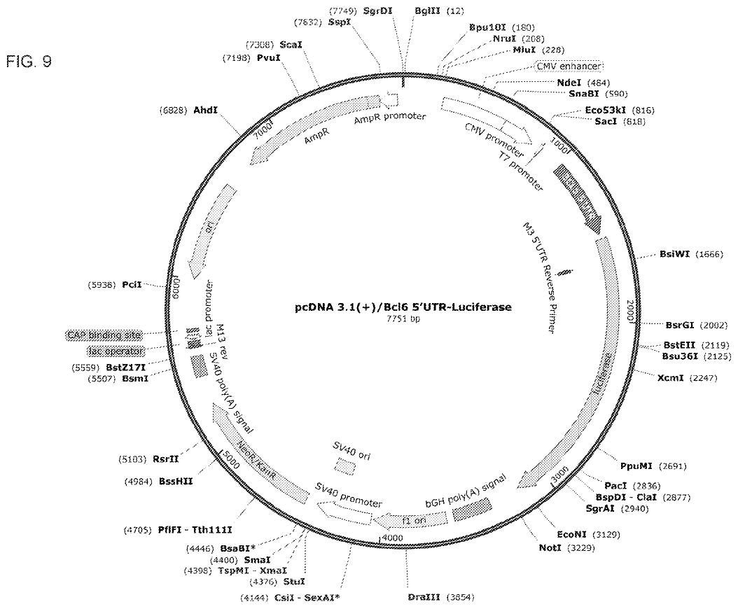

The constructs are made according to standard procedures. From 5′ to 3′, the expression cassette may include: (a) a promoter, (b) a 5′-UTR sequence of Tables 1-3, (c) on open reading frame (ORF), (d) optionally, a reporter, (e) a 3′ UTR, and (f) a polyadenylation (polyA) signal. An exemplary construct expressing a heterologous transgene in shown in FIG. 9.

In some embodiments, the promoter is a cytomegalovirus (CMV) promoter. In some embodiments, the 5′-UTR sequence further includes a non-canonical start site (e.g., a non-AUG start site of Table 3). In some embodiments, the ORF encodes a polypeptide (e.g., a toxin, inhibitor, or an antigen). In some embodiments, the OFF encodes a cell cycle or differentiation gene that is poorly expressed in chemoresistant cancers. A skilled practitioner would understand that “a cell cycle or differentiation gene that is poorly expressed in chemoresistant cancers” refers to a gene associated with cell cycle or differentiation that is known to be inhibited in chemoresistant cancer samples relative to type-matched cancer samples. In some embodiments, the cell cycle or differentiation gene that is poorly expressed in chemoresistant cancers is ID1 or MT1G. In some embodiments, the polyA signal is a bovine growth hormone (bGH) polyA signal.

Such constructs may be activated or arrested by triggering or inhibiting the integrated stress response (ISR) pathway marked by eIF2α phosphorylation. ISR may be triggered by transfecting a chemoresistant cancer cell or chemoresistant cancer with a construct of the disclosure, or by administering to the chemoresistant cancer cell or chemoresistant cancer a stressor. In some embodiments, the stressor is polyinosinic-polycytidylic acid (poly(I:C)). In some embodiments, the 18R pathway may be inhibited by administering ISRIB, trazodone, or metformin to the chemoresistant cancer cell or chemoresistant cancer.

### Antisense

Antisense constructs are generated according to any standard methods known in the art. Such constructs are typically directed to (for example, targeting) any of the 5′-UTRs or non-canonical start sikas, or both, as described herein. These constructs accordingly inhibit gene expression of molecules including such motifs. Typically, an antisense construct includes 10 to 100 (e.g., from 10 to 20, 15 to 20, 20 to 90, 25 to 80, 30 to 70, 35 to 60, 40 to 50, or 45 or 50) nucleotides.

In some embodiments, the construct includes an antisense RNA. In some embodiments, the construct encodes on antisense RNA. In some embodiments, the antisense RNA targets the 5′-UTR region of a gene in Tables 1-3. In some embodiments, the antisense RNA targets a non-canonical start site region including a poor Kozak consensus sequence (e.g., a sequence deviated from SEQ ID NO: 98). In some embodiments, the antisense RNA includes any of the sequences in Table 4.

TABLE 4
Antisense Sequences

| SEQ | | |
|---|---|---|
| ID NO: | Description: | Sequence: |
| 1 | shRNA, Rplp0-1 | ATCCGTCTCCACAGACAAG |
| 2 | shRNA, Rplp0-3 | TGTTTTGCAAATAAAACTG |
| 3 | shRNA, Rplp0 | TGAGCATGTTCAGCAGCGT |
| 4 | shRNA, Rplp0-2 | TGGACTTCCAGGTCGCCCT |
| 5 | shRNA, Rplp0 | TGCGCATCATGGTGTTCTT |
| 6 | CD47 siRNA | 5-TACTGGCCTTGGTTTAA |
| | | TTCTCGAGAATTAAACCAA |
| | | GGCCAGTAGC-3 |
| 7 | CD47 siRNA | 5-CUAUGAGACCCUUACGU |
| | | GAUUGUUA-3 |
| 8 | CD47 siRNA | 5-GCACAUGCAUCUUCUGU |
| | | AUGGACAA-3 |
| 9 | CD47 siRNA | 5-GCAUGGCCCUCUUCUGA |
| | | UU-3 |
| 10 | CD47 siRNA | 5-GUACAGCGAUUGGAUUA |
| | | AC-3 |
| 11 | CD47 siRNA | 5-CAGAGAAGGUGAAACGA |
| | | UC-3 |
| 12 | CD47 siRNA | 5-UAACUGAAGUGAAGUGA |
| | | UG-3 |

In some embodiments, the antisense RNA is at least 75% complementary to a target nucleic acid (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to the target nucleic acid). “Percent (%) complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B.

It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The terms “short interfering RNA” and “siRNA” refer to an inhibitory polynucleotide containing double stranded nucleic acid in which each strand comprises RNA, RNA analog(s) or RNA and DNA. Typically, the antisense strand of the siRNA is sufficiently complementary with the target sequence of the target gene/RNA. siRNA molecules operate within the RNA interference pathway, leading to inhibition of mRNA expression by binding to a target mRNA (e.g., a 5′-UTR RNA, as described herein) and degrading the mRNA through Dicer-mediated mRNA cleavage.

The terms “short hairpin RNA” and “shRNA” refer to an inhibitory polynucleotide containing single-stranded RNA of 50 to 100 nucleotides that forms a stem-loop structure in a cell, which contains a loop region of 5 to 30 nucleotides, and long complementary RNAs of 15 to 50 nucleotides at both sides of the loop region, which form a double-stranded stem by base pairing between the complementary RNA sequences; and, in some cases, an additional 1 to 500 nucleotides included before and after each complementary strand forming the stem. For example, shRNA generally requires specific sequences 3′ of the hairpin to terminate transcription by RNA polymerase. Such shRNAs generally bypass processing by Drosha due to their inclusion of short 5′ and 3′ flanking sequences. Other shRNAs, such as “shRNA-like microRNAs,” which are transcribed from RNA polymerase II, include longer 5′ and 3′ flanking sequences, and require processing in the nucleus by Drosha, after which the cleaved shRNA is exported from the nucleus to cytosol and further cleaved in the cytosol by Dicer. Like siRNA, shRNA binds to the target mRNA in a sequence specific manner, thereby cleaving and destroying the target mRNA, and thus suppressing expression of the target mRNA.

Such siRNAs and shRNAs may be synthetically generated.

### Modifications

It is contemplated that for any of the RNA molecules (e.g., 5′-UTRs) disclosed herein may be used either alone or in a modified form. Typically, modifications to the RNA molecules are introduced to optimize the molecule's efficacy or biophysical properties (e.g., increasing serum stability or circulating half-life, increasing thermal stability, enhancing transmembrane delivery, reduce immunogenicity of the RNA and/or targeting to a particular location or cell type).

Such modification is achieved by systematically adding or removing linked nucleosides to generate longer or shorter sequences. Further modifications include the incorporation of, for example, one or more alternative nucleosides, alternative 2′ sugar moieties, and/or alternative internucleoside linkages.

### Nucleoside Modifications

Modification of the RNA molecules described herein include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino 8-thio, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-dezaguanine and 7-deazaadenine, and/or 3-deazaguanine and 3-deazaadenine. RNA molecules may also include nucleobases in which the purine or pyrimidine base is replaced with other heterocycles, for example 7-deaza-adenine, 7-deazaguanosine, 2-aminopyridine, and/or 2-pyridone. Further modifications may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

### Sugar Modifications

Modifications of RNAs also include one or more of the following 2′ sugar modifications: 2′-O-methyl (2′-O-Me), 2′-methoxyethoxy (2′-O—CH2CH2OCH3, also known as 2′-O-(2-methoxyethyl) or 2′-MOE), 2′-dimethylaminooxyethoxy. i.e., a O(CH2)2ON(CH3)2 group, also known as 2′-DMAOE, and/or 2′-dimethylaminoethoxyethoxy (also known in the art as 2′-O-dimethylamino-ethoxy-ethyl or 2′-DMAEOE). i.e., 2′-O—CH2OCH2N(CH3)2. Other possible 2′-modifications that modify the RNAs include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH2CH2CH2NH2), allyl (—CH2-CH═CH2), —O-allyl (—O—CH2-CH═CH2) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Internucleoside Linkage Modifications

Modifications of RNAs molecules may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalklyphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### Transfection

Any of the aforementioned RNAs may be transfected, according to standard methods, into a cell in vivo, in vitro, or ex vivo. RNAs may be transfected, for example, into an appropriate cell. In some embodiments, transfection of one or more RNAs may be mediated by a liposome, an extracellular vesicle (e.g., an exosome), or a lipid nanoparticle (LNP). Any cell transfected with one or more of the RNAs is referred herein as an engineered cell and typically includes a heterologous 5′-UTR.

Any transfected cells (e.g., an engineered cell) may be transplanted into a subject (e.g., a human). Cells to be transplanted may have an autologous or allogenic origin.

The RNA molecules (e.g., in an unmodified or modified form) described herein may be formulated into various Compositions (including a pharmaceutical composition) for administration to a subject in a biologically compatible form suitable for administration in vivo. For example, the RNA molecules including 5′-UTR molecules described herein may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012. 22nd ed. and in The United States Pharmacopeial Convention, The National Formulary, United States Pharmacopeial, 2015, USP 38 NF 33).

Although the descriptions of pharmaceutical compositions provided herein are principally directed to pharmaceutical compositions which are suitable for administration t humans, it will be understood by the skilled artisan that such compositions are generally suitable for administration to any other animal, e.g., no non-human animals, e.g., non-human mammals. Modification of pharmaceutical compositions suitable for administration to humans no render compositions suitable for administration to various animals is well understood, and the ordinarily skilled veterinary pharmacologist can design and/or perform such modification with merely ordinary, if any, experimentation. Subject to which administration of the pharmaceutical compositions is contemplated include, but are not limited to, humans and/or other primates and mammals.

The 5′ UTR and pharmaceutical compositions described herein may contain at least one 5′-UTR molecule. As a non-limiting example, the formulations may further contain more than one 5′-UTR molecules. The 5′-UTR and pharmaceutical compositions described herein may be loaded into a carrier such as an extracellular vesicle (e.g., an exosome), liposome, or a lipid nanoparticle (LNP) according to standard methods known in the art. Such exemplary carriers are now described.

### Extracellular Vesicles

In one embodiment, pharmaceutical compositions of RNA molecules including a 5′-UTR include extracellular vesicles. Appropriate extracellular vesicles may be prepared using standard methods. In some embodiments, the extracellular vesicles may be exosomes. Exosomes produced from cells can be collected from the cell culture medium by any suitable method. Typically, a preparation of exosomes can be prepared from cell culture or tissue supernatant by centrifugation, filtration or combinations of these methods. For example, using standard methods, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 micrometer filter), gradient ultracentrifugation (for example, with sucrose gradient) or a combination of these methods. Exosomes are loaded with exogenous RNAs including a 5′-UTR, according to standard methods, for systemic delivery to a subject (e.g., a human patient).

### Liposomes

In one embodiment, pharmaceutical compositions of RNA molecules including a 5′-UTR include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis.

Liposomes may contain a low or a high pH in order to improve the delivery of the pharmaceutical formulations. Liposomes are loaded with exogenous 5′-UTR, according to standard methods, for systemic delivery to a subject (e.g., a human patient).

### Lipid Nanoparticles (LNPs)

In one embodiment, pharmaceutical compositions of RNAs including a 5′-UTR include lipid nanoparticles (LNPs). For example, an RNA molecule including a 5′-UTR including a BCL6 5′-UTR may be formulated in a lipid nanoparticle such as those described in International Publication No. WO2012170930, herein incorporated by reference in its entirety. As a non-limiting example, LNP formulations may contain cationic lipids, distearoylphosphatidylcholine (DSPC), cholesterol, polyethylene glycol (PEG), R-3-[(ω-methoxy poly(ethylene glycol)2000)carbamoyl)]-1,2-dimyristyloxl-propyl-3-amine (PEG-c-DOMG), distearoyl-rac-glycerol (DSG) and/or dimethylaminobutanoate (DMA). As a non-limiting example, 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypoly ethylene glycol) or PEG-DPG (1,2-Dipalmitoyl-sn-glycerol, methoxypolyethylene glycol). The cationic lipid may be selected from any lipid known in the art such as, but not limited to, (6Z,9Z,28Z,31 Z)-heptatriacont-6,9,28,31-tetraene-19-yl 4-(dimethylamino) butanoate (DLin-MC3-DMA), 1,2-dilinoleyloxy-n,n-dimethyl-3-aminopropane (DLin-DMA), C 12-200, and N,N-dimethyl-2,2-di-(9Z,12Z)-9,12-octadecadien-1-yl-1,3-dioxolane-4-ethanamine (DLin-KC2-DMA). LNPs are loaded with exogenous 5′-UTR, according to standard methods, for systemic delivery to a subject (e.g., a human patient).

### Administration

Any of the molecules described herein may be administered according to standard techniques.

For example, a molecule including the 5′-UTR described herein may be administered in unmodified or modified form and such forms may, if desired, be formulated into a composition (e.g., a pharmaceutical composition including an exosome, a liposome, or a nanoparticle) for administration to a subject in a biologically compatible form suitable for administration in vitro, in vivo, or ex vivo. In general, a suitable daily dose of one or more of the molecules including the 5′-UTR described herein will be an amount which is the lowest dose effective to produce a therapeutic effect. The molecules including the 5′-UTR described herein may be administered by injection, e.g., intravenous, intramuscular, intraperitoneal, or subcutaneous. For example, the molecules including the 5′-UTR described herein can be systemically administered to a subject via intravenous injection. Alternatively, the 5′-UTR described herein may be administered by injection transfection, such as transfection of in vitro or ex vivo cells.

Furthermore, any of the antisense constructs described herein may be similarly administered. Such methods are known in the art.

### Treatment

Any of the aforementioned RNA molecules including any of the 5′-UTRs described herein, engineered cells (e.g., cells transfected with 5′-UTRs), or compositions (e.g., pharmaceutical compositions) can be used for the treatment of a subject (e.g., a human) with a chemoresistant cancer or a brain disease. The cancer may be a leukemia, a lung cancer, a breast cancer, an ovarian cancer, a head and neck cancer, an esophageal cancer, a cervical cancer, a uterine cancer, a stomach cancer, a bladder cancer, a prostate cancer, a urothelial cancer, a pancreatic cancer, or a brain cancer. In some embodiments, the cancer has a 3q-26 locus chromosomal amplification.

In some embodiments, any of the aforementioned RNA molecules may be prepared and administered in the form of a nucleic acid based vaccine (e.g., a RNA vaccine). In some embodiments, the nucleic acid based vaccine encodes an RNA molecule described herein. In some embodiments, the nucleic acid based vaccine includes the sequence of an RNA molecule described herein.

In some embodiments, the RNA molecule is an antisense molecule, which is administered to treat a cancer (e.g., a chemoresistant cancer), for example by targeting a 5′-UTR described herein. In other embodiments, the antisense molecule targets both the 5′-UTR and a non-canonical start site, as described herein.

### Examples

Quiescent leukemic cells survive chemotherapy, with translation changes. Our data reveal that fragile X mental retardation-related protein 1 (FXR1) (M. C. Siomi, et al., Mol. Cell. Biol. 16, 3825-3832 (1996); R. Mazroui, et al., Hum. Mol. Genet. 11, 3007-3017 (2002)), a protein amplified in several aggressive cancers, is elevated in quiescent and chemo-treated leukemic cells and promotes chemosurvival. This indicates undiscovered roles for this RNA- and ribosome-associated protein in chemosurvival. We find that FXR1 depletion reduces translation, with altered rRNAs, snoRNAs, and ribosomal proteins (RPs). FXR1 regulates factors that promote transcription and processing of ribosomal genes and snoRNAs. Ribosome changes in FXR1-overexpressing cells, including RPLP0/uL10 levels, activate eIF2α kinases. Accordingly, phospho-eIF2α increases, enabling selective translation of survival and immune regulators in FXR1-overexpressing cells. Overriding these genes or phospho-eIF2α with inhibitors reduces chemosurvival. Thus, elevated FXR1 in quiescent or chemo-treated leukemic cells alters ribosomes that trigger stress signals to redirect translation for chemosurvival.

As is described in detail below, in this study, we investigated the changes in translation, and the role of FXR1, in G0 and chemosurviving AML cells. Our findings demonstrate that as in serum-starved G0 cells that are chemoresistant, FXR1 increases in therapy-surviving AML cells. Consistently, we find that FXR1 depletion reduces chemosurvival, while FXR1 overexpression promotes chemosurvival. Our data reveal that FXR1 associates with ribosome regulators and alters ribosome components. These ribosomal changes trigger stress signaling via eIF2α kinase activation that causes eIF2α phosphorylation. This reduces canonical translation and permits translation of specific pro-survival and immune genes, leading to chemotherapy and immune survival. Pharmacological inhibition of this specific translation, or of translated pro-survival genes, suppresses chemosurvival, indicating new avenues to therapeutically target refractory AML.

As non-canonical translation of these pro-survival genes is a common feature of chemoresistant cells, FXR1-based therapies show broad promise for other types of cancers, including acute myeloid leukemia, lung cancer, breast cancer, ovarian cancer, head and neck cancer, esophageal cancer, cervical cancer, uterine cancer, stomach cancer, bladder cancer, metastatic prostate cancer, urothelial cancer, and other cancers that have the 3q-26 locus chromosomal amplification where FXR1 is located, including but not limited to pancreatic cancers, and gliomas.

We now describe the results of our studies.

### Results

### FXR1 Increases in Serum-Starved G0 and Cytarabine Chemosurviving THP1 Cells

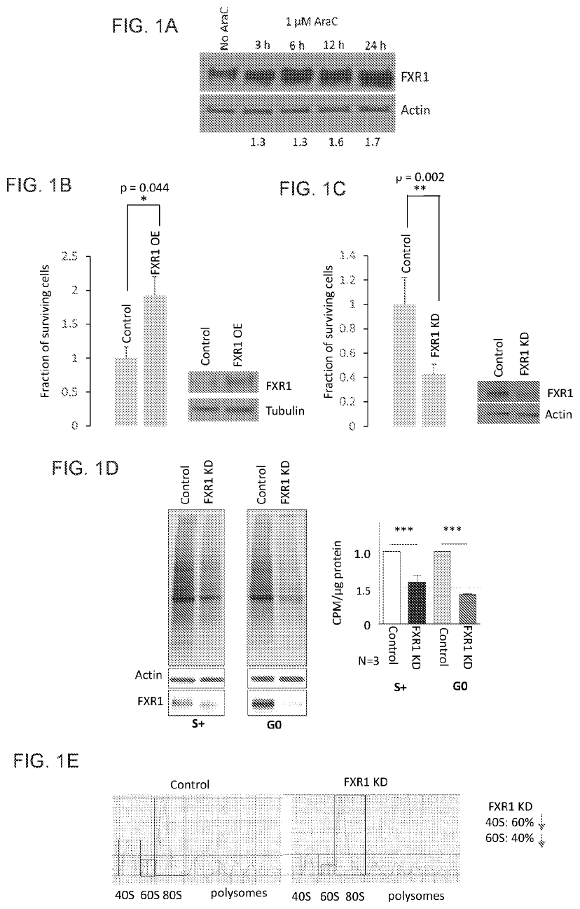

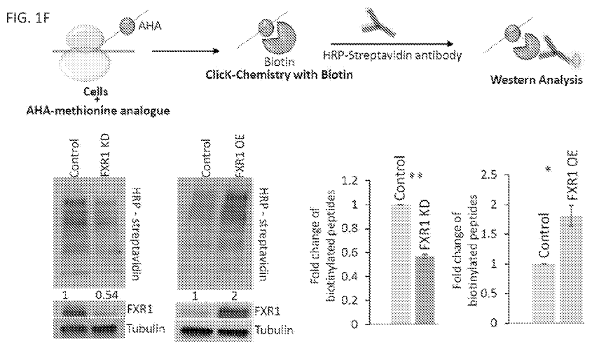

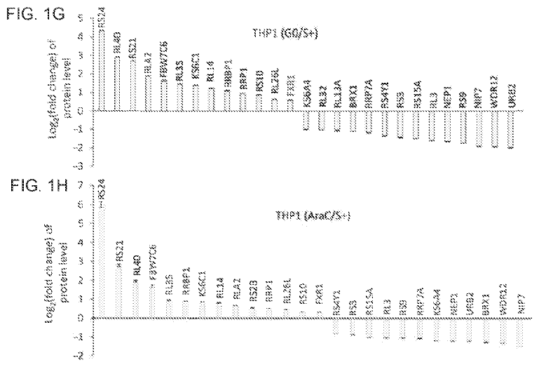

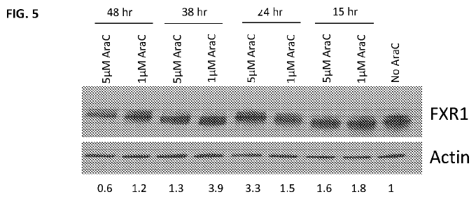

We tested FXR1 levels in Cytarabine (AraC) chemotherapy treated cells where a target of FXR1, TNFα, is promoted and is required for AraC survival. We find that FXR1 increases in AraC treated surviving THP1 cells (AraC, FIGS. 1A and 5A) and on serum-starvation (G0) of THP1 cells that are chemoresistant. This was also observed upon AraC treatment or serum-starvation of a number of AML cell lines, including THP1 and NOMOI cell lines, compared to normal monocytes. FXR1 and phospho-eIF2α levels in multiple AML cell lines treated with AraC, and AML (M5 subtype similar to THP1) patient samples treated with AraC, and in serum-starved AML M5 patient samples were compared to normal monocytes. Data were averaged from three replicates +/− SEM. Taken together, these data indicated that FXR1 levels increase in chemoresistant leukemia cells. These data further revealed an increase of FXR1 in chemotherapy treated (chemosurviving) cells.

### FXR1-Overexpressing Cells are Resistant to AraC Chemotherapy

To investigate whether FXR1 plays a role in chemosurvival of G0 and AraC treated cells where it increases, we used a previously constructed FXR1 depleted THP1 cell line that is stably transduced with an shRNA that depletes all FXR1 isoforms (FXR1 KD) upon doxycycline induction, compared to a parallel control shRNA expressing cell line (control). These cells are induced with doxycycline to express the shRNA for 3 days to effectively deplete FXR1. We also constructed a THP1 cell line that constitutively overexpresses FXR1 (FXR1 OE) compared to a vector control cell line. Western blot analysis show that FXR1 was effectively depleted or overexpressed (FIG. 1B-C, Western blots, graphs below). These were treated with AraC to test the impact of FXR1 levels on chemosurvival. Consistent with its increase in AraC-surviving cells, we find that FXR1 overexpression promotes while FXR1 depletion reduces chemosurvival (FIG. 1B-C graphs). These data indicate that FXR1 promotes chemosurvival of THP1 cells.

FXR1 Depletion Decreases, while Overexpression Promotes General Translation

We next explored the mechanism of how FXR1 may promote chemosurvival. To investigate the effect of FXR1 on general translation, we used L-homopropargylglycine (HPG), an amino acid analog of methionine containing an alkyne moiety that can be biotinylated by click-it chemistry (ThermoFisher), in parallel with 35S Met incorporation analysis to label nascently translated proteins in FXR1 knockdown cells followed by SDS-PAGE separation and phosphorimager quantitation or scintillation counter quantitation of the total levels of nascently labeled proteins. FXR1 depletion decreased the levels of protein synthesis as observed from the 35S Met incorporation by 30% decrease in serum conditions and upto 50% decrease in serum-starved cells (FIG. 1D). Conversely, FXR1 overexpression increases nascent translation by 1.7 fold compared to control vector cells (FIG. 1F). These data indicate that FXR1 is needed for translation.

### FXR1 Depletion Leads to Decreased Ribosome Subunits

To test whether the significant decrease in translation was due to perturbations in ribosome levels, we conducted polysome analysis in FXR1 depleted cells compared to control shRNA cells (FIG. 1E). We find that both 60S and 40S subunits decreased significantly (40% and 60% respectively, FIG. 1E), indicating reduced ribosome subunits in FXR1 defective cells.

FXR1 Depletion Decreases, while Overexpression Increases rRNAs

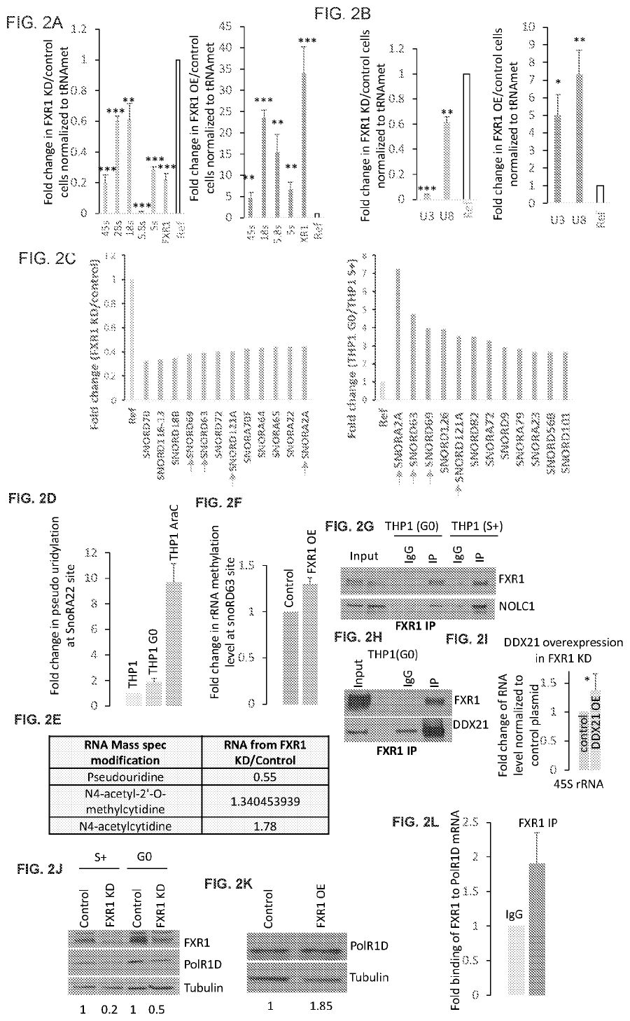

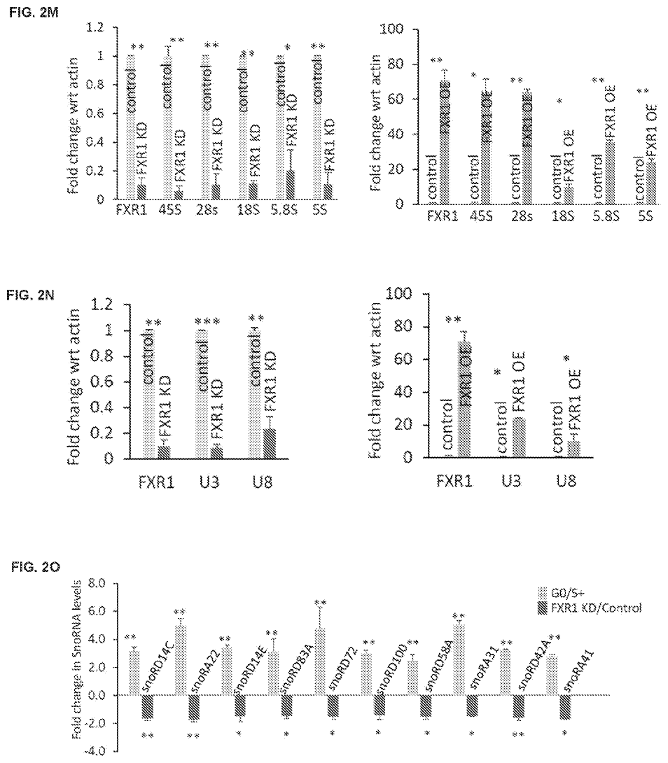

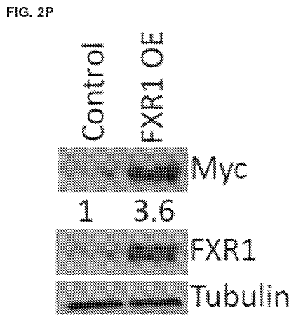

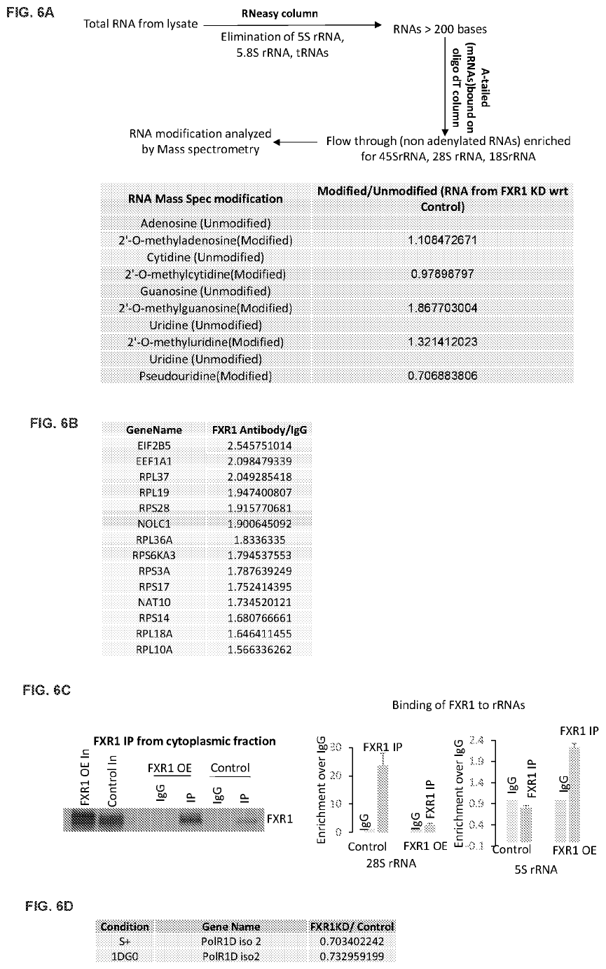

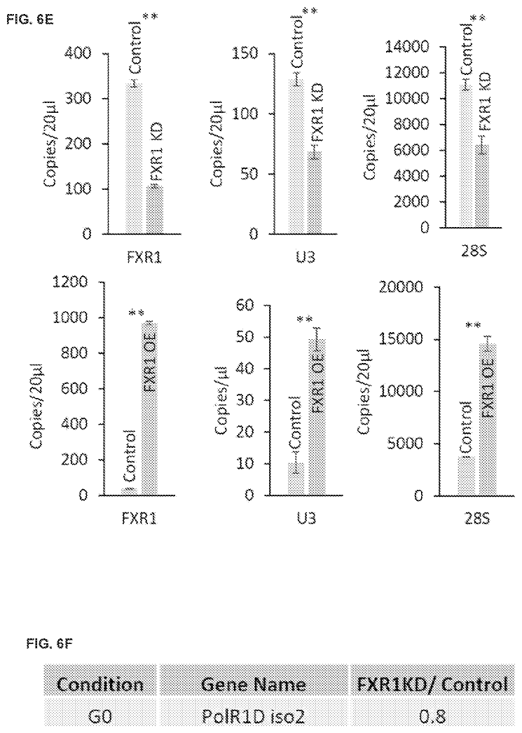

Given the decrease in ribosome subunits, we investigated whether FXR1 levels alter ribosome biogenesis. Ribosome biogenesis includes rRNA transcription and processing, their modification by small nucleolar RNAs (snoRNAs), ribosomal proteins and their assembly, with several regulators that are involved in rRNA and ribosome gene transcription, localization or processing. Analysis of ribosomal RNAs (rRNAs) by qPCR revealed a significant decrease in 28S, 18S and 5.8S rRNA levels upon FXR1 depletion, compared to control shRNA cells (FIGS. 2A, 6E). Conversely, 28S, 18S, and 5.8S rRNAs increase upon FXR1 overexpression compared to control vector cells. These effects include both transcriptional and processing effects as 45S precursor rRNA was also reduced upon FXR1 depletion, indicating a decrease in ribosome biogenesis. This decrease is not just due to Pol I transcription as the 5S rRNA that is transcribed by Pol III, is also decreased (FIG. 2A). In contrast, FXR1 overexpression increased all rRNA levels (FIG. 2A), consistent with the previously noted increase in general translation upon overexpression of FXR1. These data are consistent with regulation of ribosome biogenesis by FXR1 depletion.

SnoRNAs Involved in rRNA Processing and Other snoRNAs that Modify rRNAs, Increase in G0 THP1 and FXR1-Overexpressing Cells, and Decrease Upon FXR1 Depletion

Ribosome biogenesis involves ribosome biogenesis regulators including snoRNAs. SnoRNAs bind RNA modification enzymes and other RNA binding proteins to form snoRNPs that are known to base pair and modify rRNAs at specific sites. Box C/D snoRNAs or snoRDs recruit Fibrillarin enzyme to cause 2′-O-methylation while Box H/ACA snoRNAs or snoRAs recruit Dyskerin enzyme to cause pseudouridylation of rRNA sites. These modifications affect ribosome interactions while specific snoRNAs involved in ribosome biogenesis, such as U3 (snoRD3) and U8 (snoRD118), are required to cleave the 45S rRNA precursor for rRNA processing into mature 18S, 28S and 5.8S rRNAs.

We first examined the snoRNAs involved in ribosome biogenesis such as snoRD3/U3 that is involved in rRNA processing along with snoRD118/U8. Global profiling previously revealed that U3 increases in G0 THP1 cells compared to proliferating, serum-grown cells. Consistently, we find that U3 and U8 are decreased upon FXR1 depletion and increased upon FXR1 overexpression (FIG. 2B, 6E). These data are consistent with the effect of FXR1 on ribosome levels and translation and on rRNA levels (FIGS. 1B-1E, 1G-1H, 2A).

Global profiling from our previously derived dataset revealed that other specific snoRNAs that modify distinct rRNA sites increase in G0 THP1 cells and many associate with FXR1 in co-immunoprecipitates. Consistently, we find these are decreased upon FXR1 depletion and increased upon FXR1 overexpression (i.e., in G0) (FIG. 2C). To test the functional outcome of FXR1 interaction on snoRNAs, we enriched rRNA (devoid of poly(A) RNA and RNAs less than 200nt) from FXR1 knockdown G0 cells and control cells and subjected them to LC MS/MS and RNA mass spectrometry (FIGS. 6B-6C).

We also analyzed CMCT based pseudouridylation and 2′-O-methylation analyses by low dNTP qPCR at the rRNA sites of specific snoRNAs targeted to be increased in G0 regulated by FXR1. We find changes in modification by the snoRNAs regulated by FXR1. For 2′-O-methylation of rRNA by a snoRD regulated by FXR1: as shown in FIG. 2D, the snoRNA snoRD63 modification site (28 S rRNA A4541m) and snoRD58A both show enhanced 2′-O-methylation in FXR1 overexpression cells, consistent with snoRD63 and snoRD58A increase in G0 cells where FXR1 increases and its decrease in FXR1 knockdown cells (FIG. 2O). For the snoRNA, snoRA22 that increases in G0 and decreases with FXR1 depletion, we find that rRNA pseudouridylation at snoRA22 sites on 28S at U4966 and U4975 increases in G0 and AraC cells (FIG. 2E). Consistently, we find that FXR1 knockdown reduces overall pseudouridylation by mass spectrometry (FIG. 2F). Other known rRNA modifications also show changes in FXR1 knockdown cells by mass spectrometry (FIG. 6A). The sites targeted by many of these FXR1 regulated snoRNAs are key for tRNA interaction and translation, indicating that FXR1 can alter translation by affecting ribosome modifications.

Together, these data indicate that FXR1 levels alter specific snoRNA levels and functions, which can affect ribosomes and translation.

FXR1 Interacts with the rRNA and snoRNA Regulator, DDX21, and Regulates PolR1D that Increases in G0 THP1 Cells

To identify how FXR1 may mediate effects on snoRNAs and on rRNA levels, we examined FXR1 immunoprecipitates for interacting regulators by in vivo crosslinking G0 THP1 cells followed by FXR1 immunoprecipitation and Tandem-Mass-Tag spectrometric analysis of co-immunoprecipitates. We find that FXR1 interacts with multiple ribosome and translation related proteins in G0 cells (FIG. 6B). Consistently, we find that in vivo crosslinking coupled FXR1 immunoprecipitation in control or FXR1 overexpression cells reveals FXR1 association with rRNAs (FIG. 6C). These data indicate that FXR1 may affect ribosome regulators through multiple associations.

We find that snoRNA and ribosome regulators DDX21 as well as NOLC1, co-immunoprecipitated with FXR1 (FIGS. 2G-2H, 6B-6C), indicating that FXR1 may affect ribosome levels through such interactions with such ribosome regulators. Consistently, overexpression of DDX21 in FXR1 KD cells, where rRNA levels decrease (FIG. 2A), partially rescued the rRNA defect by increasing levels of 45S rRNA (FIG. 2N). These data indicate that FXR1 levels may affect the functions of a key ribosome gene transcription and snoRNA regulator, DDX21, which could lead to the alterations in the ribosome observed. Additionally, FXR1 is known to bind and regulate the 3′UTR of c-MYC mRNA, a major ribosome gene transcription regulator that affects all three polymerases and regulates ribosome biogenesis. Consistently, we find that c-MYC increases upon FXR1 overexpression (FIG. 2P), which could lead to enhanced ribosome biogenesis. Since DDX21 associates with the Pol I complex, and FXR1 affects both Pol I transcript 45S rRNA and Pol III transcript 5S rRNA, we hypothesized that a common Pol I and Pol III component may be affected by FXR1 and G0 chemoresistant cell conditions. Therefore, we tested whether common Pol I and Pol Ill components are increased in G0 and are affected in FXR1 depleted or overexpressed cells. POLR1D is an essential component of both Pol I and Pol Ill complexes and is needed for DDX21 to locate to the nucleolus for both 45S and 5S rRNA production. Our mass spectrometry G0 and AraC treated cell dataset reveal that POLR1D isoform 2 (iso2) increases in G0 cells that are chemoresistant and show FXR1 increase (FIG. 6D), but is reduced upon FXR1 depletion (FIG. 6F). Consistently, we find that POLR1D is reduced and overexpressed with FXR1 knockdown or overexpression respectively (FIG. 2J-2K). Accordingly, we find that POLR1D iso2 RNA associates with FXR1 (FIG. 2L), indicating that FXR1 associates with the mRNA of POLR1D to promote the levels of this ribosome gene transcription regulator. Thus, FXR1 regulation of the levels of a common factor of the Pol I and Pol Ill complexes, POLR1D, can affect levels of all rRNAs observed in FIG. 2A. Together, these data suggest that FXR1 interacts with or regulates the levels or functions of specific ribosome biogenesis factors to alter ribosomes in G0 and AraC cells where FXR1 increases.

### Ribosome Complexes in G0 or AraC Treated Cells, Migrate Distinctly Compared to Proliferating Cells, but Similarly to Ribosome Complexes in FXR1 Overexpressing Cells

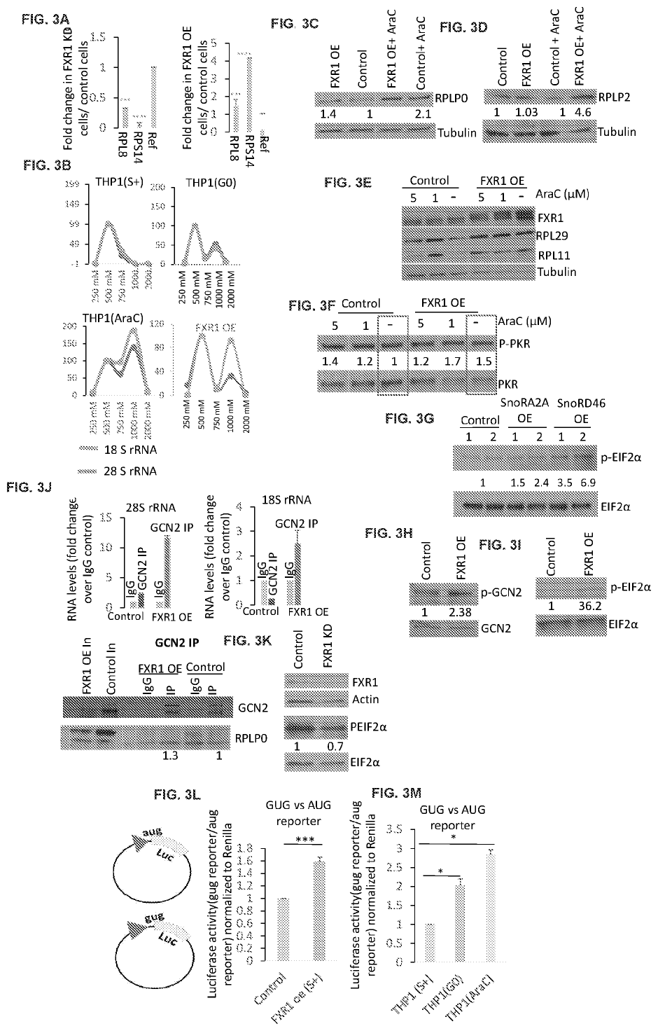

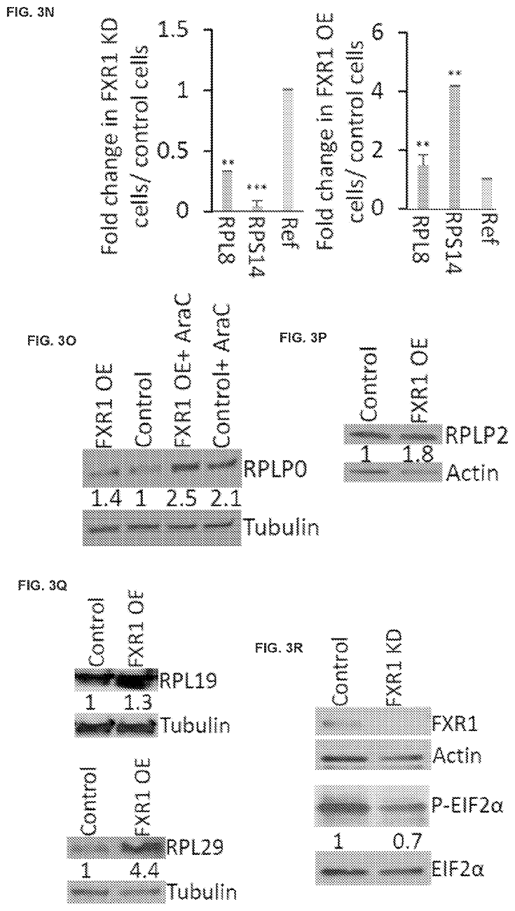

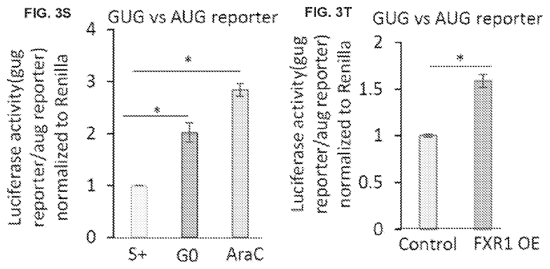

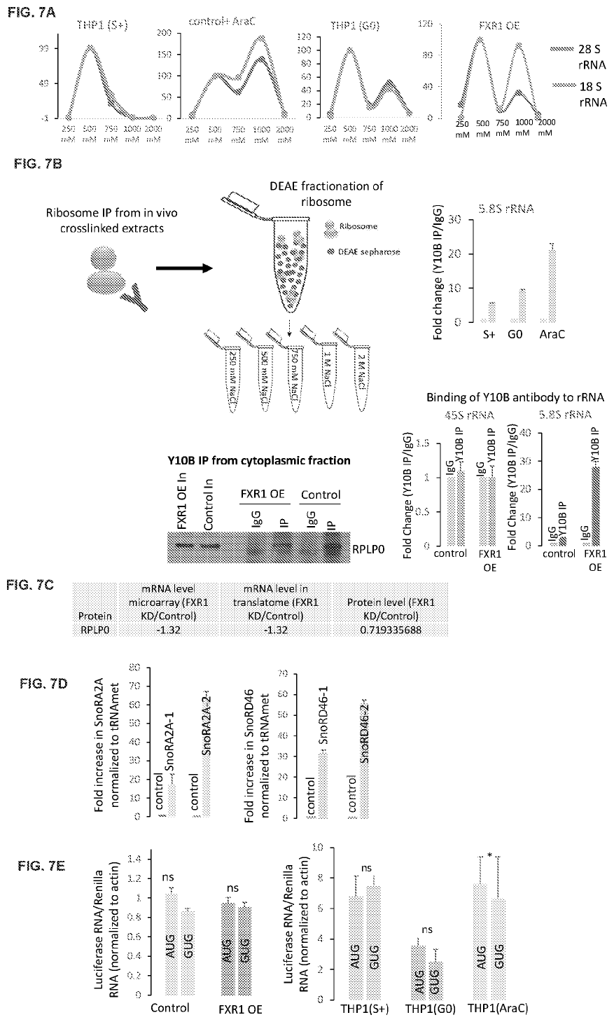

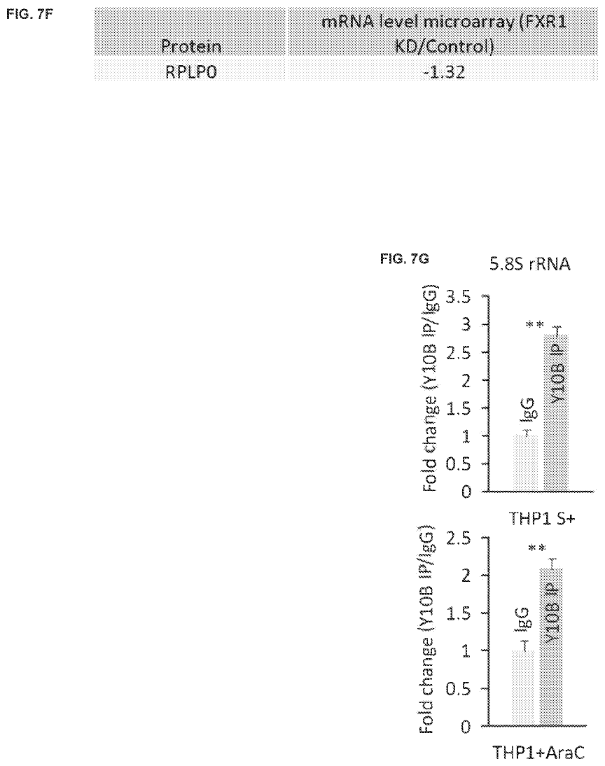

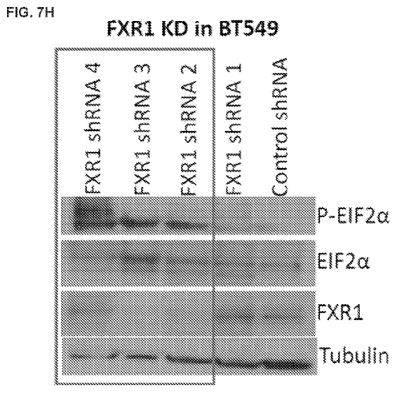

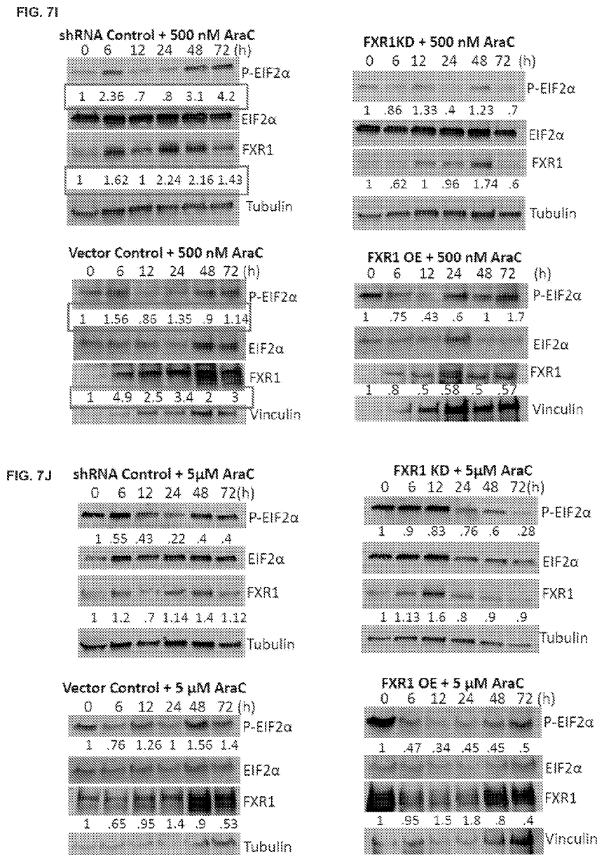

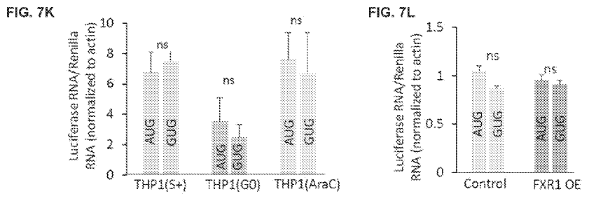

Given that snoRNAs and rRNAs as well as their processing regulators are associated with or regulated by FXR1 in G0, we examined the other main ribosome component, ribosomal protein (RP) levels. Many RPs contribute to rRNA processing and RP genes are regulated by DDX21 and some RP genes were shown previously to interact with FXR1 as well as in G0 cells (FIG. 6B). Proteomic and RNA analyses reveal that specific RPs show increase in G0 and AraC treated cells (FIG. 3A), decrease upon FXR1 depletion (FXR1 KD cells compared to control shRNA cells), or are enhanced upon FXR1 overexpression (FXR1 OE cells compared to control vector cells), as seen by qPCR (FIG. 3N, RPL8, RPS14), Western blot (FIGS. 3O-3Q, RPLP0, P2, L11, L29), or by profiling analyses (FIG. 7A). These data indicate ribosome changes in cells with elevated FXR1 levels, as in G0 and AraC-treated cells, can alter ribosome complexes and translation output.

Given rRNA processing, RP level, and modification changes induced in G0 by FXR1 overexpression, we analyzed ribosome complexes formed in AraC treated and serum-starved G0 cells compared with proliferating cells, as well as in FXR1 overexpression cells compared to vector control cells. These cells were first subject to in vivo formaldehyde crosslinking to freeze in vivo ribosome complexes. To identify changes in ribosome complexes, crosslinked extracts were separated by increasing salt concentrations over an anionic column (DEAE). Those fractions with such in vivo crosslinked complexes that contained both 18S and 28S rRNAs, depicting small and large subunits, were examined as composite 80S ribosome complexes. We find two distinct peaks bearing such ribosome complexes in serum-starved G0 and AraC-treated cells compared to one peak in proliferating, untreated, serum-grown cells (FIG. 7A).

We next examined purified ribosomes from G0 and AraC treated cells compared to untreated, serum-grown cells, and FXR1 overexpression cells compared to vector control cells. These cells were first subject to in vivo formaldehyde crosslinking to freeze in vivo ribosome complexes. Ribosome complexes were purified by Y10B, an antibody that recognizes 5.8S complexes in assembled 80S complexes and ribosomes. The purification was verified by qPCR analysis of enrichment of 5.8S rRNA in Y10B immunoprecipitates compared to IgG control immunoprecipitates (FIG. 7B) but not unprocessed 45S rRNA, as well as by Western analysis of ribosomal protein RPLP0 in Y10B immunoprecipitates compared to IgG controls. To test whether ribosomes migrate distinctly in FXR1 overexpressing cells, the Y10B immunoprecipitates were fractionated over DEAE to examine which fractions showed both 18S and 28S rRNAs depicting small and large subunit rRNAs, as composite 80S ribosome complexes, by qPCR analysis. As shown, a second complex is observed at 1M salt in G0 and AraC treated cells compared to untreated, serum-grown cells (FIG. 3B), consistent with the ribosome component changes observed in these conditions in FIGS. 2, 3A and in 7A. Interestingly, we see a similar pattern of two distinct peaks of ribosome complexes in FXR1 overexpression cells compared to vector control serum-grown cells, where the additional 1M salt fraction complex seen in G0 & AraC treated cells is also observed in FXR1 overexpression cells compared to the single lower salt peak in vector control cells (FIG. 3B). While this could reflect different ribonucleoproteins (RNPs) associated with the ribosome, causing differential complex migration, it indicates that ribosomes are differentially bound or comprised in FXR1 overexpression cells that migrate in fractions that are also similarly observed in G0 and AraC-treated cells but not in untreated, serum-grown proliferating cells. These data indicate that in FXR1 overexpression conditions: in FXR1 overexpression cells, serum-starved G0 cells, and AraC resistant cells, ribosomal complex changes are observed.

### G0 Chemoresistant Cells and FXR1 Overexpressing Cells Alter Ribosomal Proteins

Consistent with the changes in ribosomal complex migration (FIG. 3B), we find that the specific RPs increased or decreased in G0 are also remarkably increased in AraC treated cells, indicating that common changes in RP composition occur in G0 cells that are chemoresistant, and AraC surviving cells. Interestingly, some of these proteins such as the P stalk proteins, are in close proximity to critical functional parts of the ribosome such as where eEF2 associates in the ribosome. Interestingly, we find many of these are decreased upon FXR1 depletion or increased in FXR1 overexpression cells that are treated with AraC. These include P stalk proteins, RPLP0 and RPLP2, and other RPs like RPL11 and RPL29 that are increased in G0 and AraC treated cells as well as in FXR1 overexpression cells or decreased in FXR1 depletion cells (FIGS. 3C-3E, 7C). These data indicate differences in the ribosome in cells with increased FXR1 levels, as in G0 and AraC treated cells, which can impact the function of the ribosome.

G0 Chemoresistant Cells and FXR1 Overexpressing Cells Alter snoRNAs and P Stalk Proteins, and Increase eIF2α Phosphorylation

G0 and AraC surviving cells where FXR1 increases, show inhibition of canonical translation with increased phosphorylation of eIF2α. This can be brought about by signaling changes that are induced by FXR1, via increase of snoRNAs, and via FXR1-induced ribosome changes in the RP P stalk proteins.

FXR1 enhances levels of several snoRNAs, which have been shown to bind the ISR kinase PKR that is a dsRNA binding protein and activate it to cause eIF2α phosphorylation. We hypothesized that increased snoRNAs by FXR1 amplification as in AraC surviving cells could, in part, lead to eIF2α phosphorylation by PKR. Consistently, we find that FXR1 overexpression cells and AraC treated cells show increased phosphorylation and activation of PKR (FIG. 3E). Therefore, we tested the impact on eIF2α phosphorylation upon overexpression of two snoRNAs that are enhanced in G0 cells and on FXR1-overexpression, snoRD46 and snoRA2A. We verified overexpression of these transfected snoRNAs by qPCR compared to a control vector (FIG. 7D). We find that overexpression of these snoRNAs leads to increased eIF2α phosphorylation, compared to expression of a control vector (FIG. 3G). This is consistent with prior studies that show that Fibrillarin upregulation that causes increased 2′-O-methylation, enables non-canonical translation that would be feasible with eIF2α phosphorylation. Thus, overexpression of FXR1 can increase snoRNAs that can activate dsRNA binding kinase, PKR, which can lead to eIF2α phosphorylation.

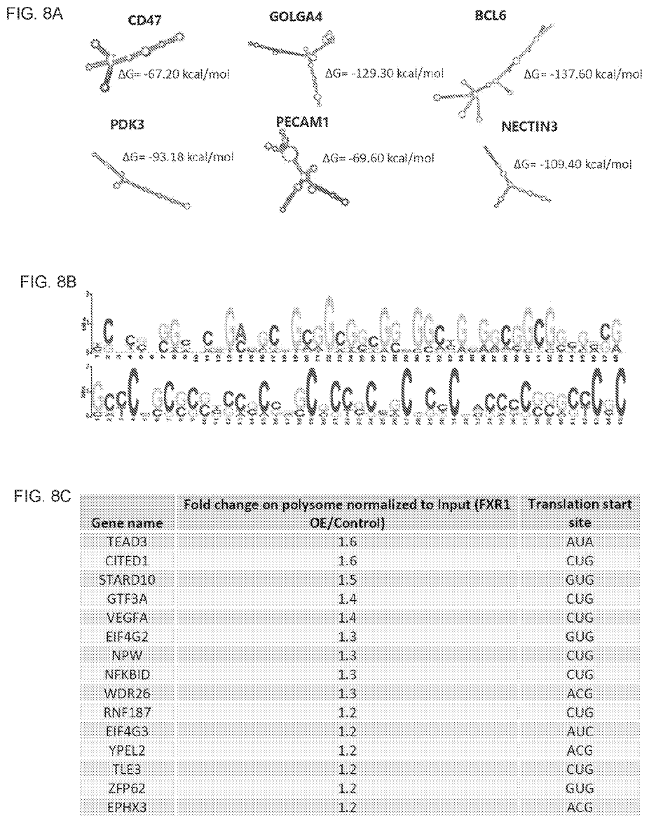

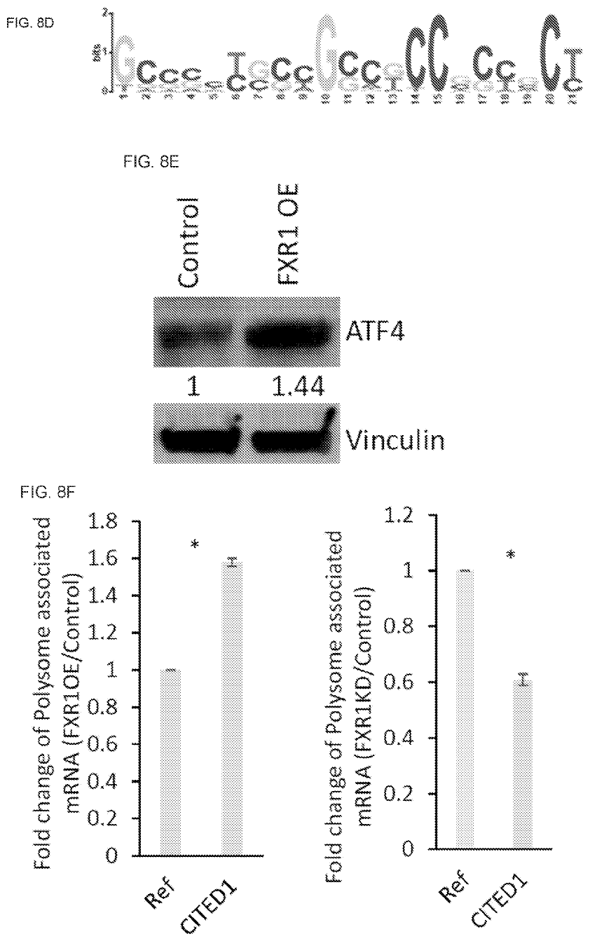

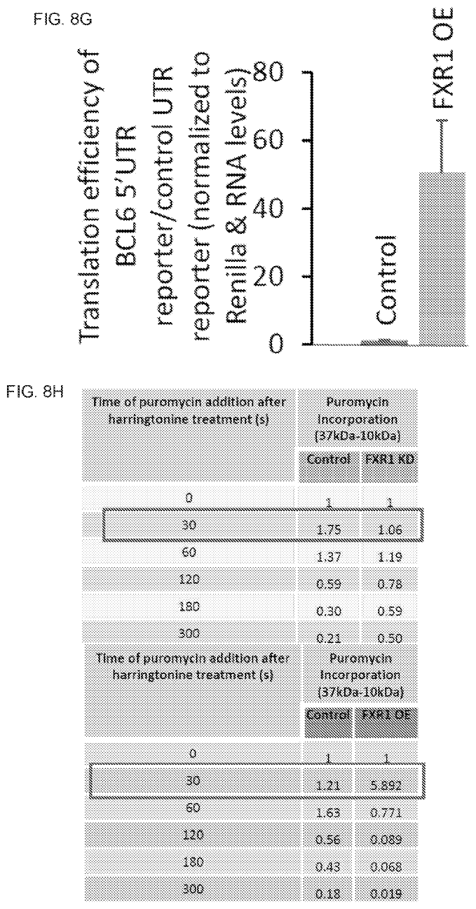

We find that RPLP0 increases in FXR1 overexpressing cells (FIG. 3C) and decreases upon FXR1 knockdown (FIG. 7C). Similarly, we find that RPLP2 increases in FXR1 overexpressing cells with AraC treatment (FIG. 3D). Consistent with the fact that increase in P stalk proteins activate GCN2 and cause eIF2α phosphorylation, we tested FXR1 overexpressing cells for GCN2 phosphorylation that marks its activation, and downstream eIF2α phosphorylation. We find that FXR1 overexpressing cells show increased GCN2 phosphorylation and thus activation (FIG. 3O), and concordantly, increased eIF2α phosphorylation (FIG. 3P). Concurrently, we find that GCN2 associates more significantly to ribosomes (graph of rRNAs) and RPLP0 in FXR1 overexpression cells, which may enable GCN2 activation and eIF2α phosphorylation (FIG. 3J). This increase in FXR1 and eIF2α phosphorylation is regulated over time of AraC treatment, but is variable at higher concentrations of AraC (FIGS. 7J-K), likely due to multiple effects including cell death, and feedback regulation of phosphatases and mTOR that affect eIF2α phosphorylation and FXR1 levels. Consistently, this is reversed in FXR1 knockdown cells where GCN2 is less associated with ribosomes (graph of rRNAs) and RPLP0; in accord, phosphorylation of eIF2α decreases upon FXR1 depletion in THP1 and other cell types (FIGS. 3J-3R, 7I). These data indicate that ribosome changes in FXR1 overexpression cells, as in G0 and AraC treated cells where FXR1 increases, is associated with stress induced eIF2α kinase activation and eIF2α phosphorylation. FXR1 overexpressing cells, similar to G0 and AraC-treated cells, promote non-canonical translation by activating stress signaling pathways. eIF2α phosphorylation inhibits canonical translation and enable non-canonical translation, including use of mRNAs with complex 5′ UTRs or start sites embedded in poor Kozak consensus regions or non-canonical start sites. In addition, such non-canonical translation been reported with increase of the snoRNA associated 2′-O-methylation enzyme, Fibrillarin. FXR1 increases in G0, and FXR1 overexpression cells have altered ribosome complexes and increased snoRNAs and altered modifications on rRNAs (FIGS. 2, 3B-C). These changes upon FXR1 increase can redirect translation: increased snoRNA modification can promote non-canonical translation, altered ribosomes can promote specific mRNA and 5′UTR translation, and phosphorylation of eIF2α can inhibit canonical translation to permit non-canonical translation. Therefore, we tested luciferase reporters with a GUG start site that would be translated by non-canonical translation, compared to a canonical AUG start site reporter (FIGS. 8G, 9A).

In G0 and in AraC surviving cells, we find that the translation of GUG reporter to that of the AUG reporter is enhanced compared to proliferating cells, indicating that non-canonical translation is enabled in these conditions with reduction of canonical AUG translation (FIG. 3L). Given that FXR1 is increased in these conditions, we asked if FXR1 overexpression affects non-canonical translation. We find that, as in G0 cells, FXR1 overexpressing cells enhance the ratio of GUG reporter translation to that of the AUG reporter, compared to vector expressing proliferating cells (FIG. 3M); these changes in luciferase expression are not seen at the RNA levels of the reporters (FIG. 7E). These data indicate that non-canonical translation is enabled by FXR1 overexpression, and changes in the ribosome seen with FXR1 increase in G0 and AraC surviving cells, may promote non-canonical translation of specific genes that enable chemosurvival.

### FXR1 Overexpression Promotes Translation of Survival Genes by Promoting Translation of Non-Canonical Start Sites

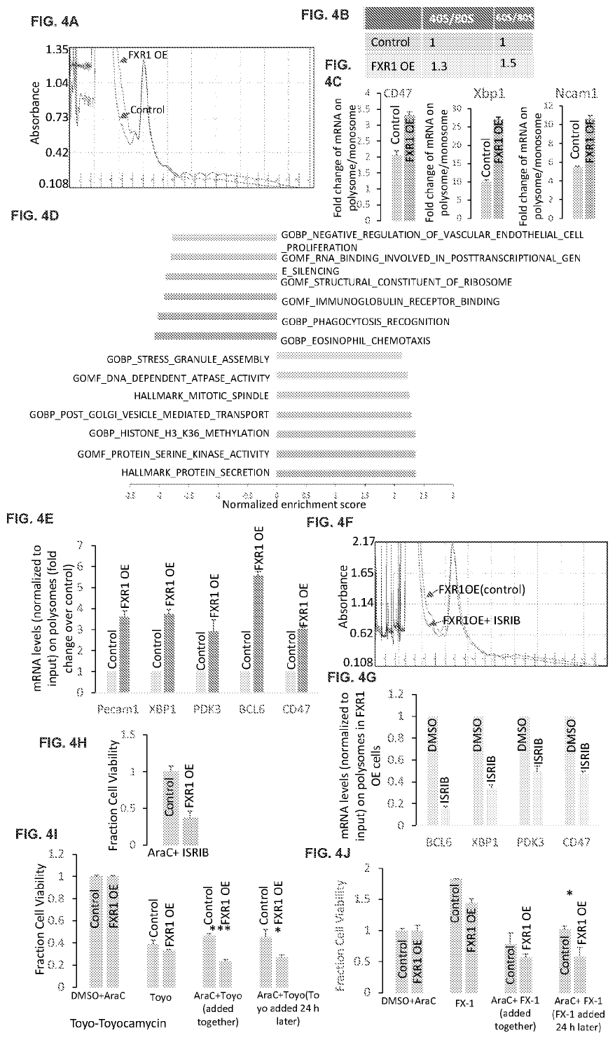

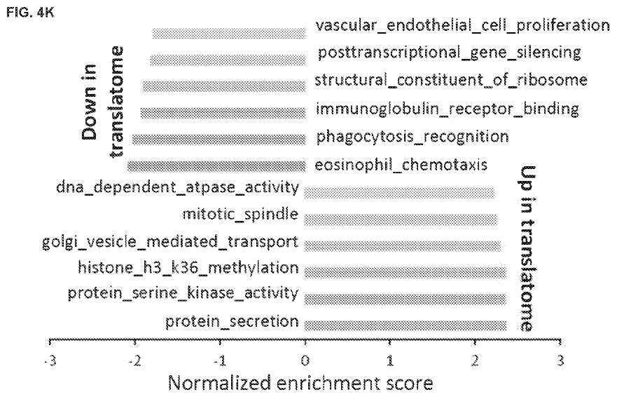

Given these data, FXR1 overexpression in G0 and AraC cells may specifically promote non-canonical translation of specific mRNAs. We therefore performed polysome analysis of FXR1 overexpressing cells compared to vector control cells (FIGS. 4A-4C). We find, as expected that the levels of subunits increase moderately in FXR1 overexpressing cells. Then, we analyzed mRNAs that are known to be increased in G0 THP1 cells but decreased upon FXR1 depletion from our previous data^{1,28}. These mRNAs were not found to be targets bound with FXR1-RNP studied previously but remained sensitive to FXR1 regulation indirectly. Furthermore, these mRNAs have G-rich or structured 5′UTRs, that would be favored by non-canonical translation conditions as in G0 and FXR1 overexpression cells where eIF2α is phosphorylated. These mRNAs include cell adhesion genes such as NCAM1, anti-tumor immune regulators such as CD47, and stress response genes such as XBP1 that are implicated in chemosurvival, observed in chemoresistant G0 cells, and in FXR1 overexpression cells.

Consistently, we find by qPCR analysis of polysome fractions compared to monosomes and input, that these mRNAs are enriched on polysomes compared to vector cells, indicating their increased translation in FXR1 overexpression cells as in G0 and AraC treated cells, which could lead to chemosurvival (FIG. 4C). To identify all mRNAs that are translated by FXR1 overexpression as in G0 and in AraC surviving cells where it increases, we conducted profiling of the polysomes of vector and FXR1-overexpression cells (FIGS. 8, 4D-E). The heavy polysomes (>2) were pooled and profiled compared to input samples to identify mRNAs that are promoted by FXR1-overexpression and increase on polysomes. We find that at least ˜10% (300 genes out of 2800) are genes that are upregulated in the FXR1 overexpression cell translatome from the increased proteome of both G0 and AraC resistant cells. Apart from pro-survival oncoproteins like BCL6, and anti-tumor immune regulators such as CD47 that we previously noted to be upregulated in G0 and AraC-treated cells (FIG. 4C), mRNAs translated upon FXR1 overexpression also include and stress response genes like XBP1, cell adhesion genes like PECAM1, signaling regulators that include PI3K-AKT-mTOR pathway, and metabolic enzymes like PDK3 that are known to promote tumor survival (FIG. 4K). These RNAs have high G-rich or structured 5′UTRs (FIG. 8) that would be favored by non-canonical translation conditions as in G0 and FXR1 overexpression cells where eIF2α is phosphorylated. For example, in G0 and AraC-surviving cells, we find that the translation of GUG reporter over that of the AUG reporter is enhanced, compared to the translation ratio in untreated, serum-grown proliferating cells (FIG. 3S), indicating that translation of unconventional start sites is enabled in G0 and AraC-treated conditions. We tested if FXR1 overexpression affects translation of non-canonical start sites, as this translation could be due to FXR1 amplification, or other factors in G0 and AraC-treated cells.

We find that, as in G0 cells, the translation of GUG reporter over that of the AUG reporter is enhanced in FXR1-overexpressing cells, compared to control vector cells, even without induction of G0 or AraC treatment (FIG. 3T). These changes in luciferase expression are not seen at the RNA level (FIG. 7K). These data indicate that translation of atypical start sites is enabled in G0 cells, AraC-treated cells, and in FXR1 OE cells. FXR1 translated mRNAs also include anti-apoptotic factors like BCL6, and kinases and regulators that include the PI3K-AKT-mTOR pathway and metabolic enzymes like PDK3 that are known to promote tumor progression (FIG. 4D). In addition, several classes of critical genes such as immune cell receptors that would be recognized by immune cells, and immune cell-attracting chemokines, are decreased in the translatome of FXR1-overexpressing cells compared to control vector cells (FIG. 4K, down in translatome genes). The suppression of immune-recognition receptors and chemokines, while inducing immune evasion receptors like CD47, indicate specific mRNA translation to survive anti-tumor immunity. These data indicate that specific gene categories are modulated by FXR1 increase to enable AML survival. These data suggest that key survival gene categories are promoted by FXR1 increase in AraC-resistant cells to enable chemosurvival.

Specific mRNA Translation in G0 Cells with Distinct 5′UTRs

To verify the translationally upregulated mRNAs in FXR1 OE cells, we performed qPCR analysis of polysome fractions that are normalized for input levels, which reveal that such mRNAs are enriched on polysomes of FXR1 OE cells compared to control vector cells (FIG. 4D)—indicating their enhanced translation when FXR1 is amplified. mRNAs that are upregulated in FXR1-overexpression translatome that overlap with increase in AraC-treated proteome, are enriched for GC-rich 5′UTRs (FIGS. 8A-D). Interestingly, several genes known to be translated by non-AUG start sites were included among the upregulated translatome in FXR1 overexpression cells and have GC-rich 5′UTRs (FIG. 8C). Such GC-rich 5′UTRs and non-canonical start sites are not preferred for conventional tRNA recruitment by eIF2 in the canonical translation mechanism. Therefore, translation of these mRNAs could be enabled by unconventional translation conditions in G0, AraC-treated, and FXR1-overexpressing cells where eIF2α is phosphorylated to reduce its activity. In accord, we find that such unconventional target genes, including those regulated by upstream open reading frames (uORFs, ATF4), and non-canonical start sites (CITED1) are increased in FXR1 OE but decreased in FXR1 KD cells, consistent with their need for eIF2α phosphorylation to reduce conventional translation (FIGS. 8E-H). To test whether these 5′UTRs contribute to specific translation in G0 cells, we constructed a reporter with the 5′UTR of BCL6, one of the translated genes in FXR1-overexpression cells that is also enhanced in G0 and AraC-treated cells. We find that the translation efficiency of BCL6 5′ UTR luciferase over a control 5′ UTR (CX) reporter (normalized for Renilla co-transfection control and RNA levels), is elevated in G0 compared to proliferating cells, and in FXR1 OE compared to control vector cells (FIG. 4E). These data indicate specific mRNA translation involve, in part, distinct 5′ UTRs that are enabled upon eIF2α phosphorylation (e.g., the 5′-UTR sequences in Tables 1-3), elicited by FXR1-mediated ribosome changes.

### Impact on Translation Mechanism

To understand the mechanism of translation, we analyzed polysomes in FXR1 depleted cells compared to control shRNA cells. As in the case of FXR1 OE cells compared to control vector cells (FIGS. 4A-4C), while specific genes are altered on polysomes upon FXR1 depletion, no significant difference was observed in polysome profiles, and in the polysome to monosome (P/M) ratio in profiles conducted in FXR1 depleted cells compared to control shRNA cells (FIGS. 4F-4G).

The P stalk proteins are at the GTPase activation center (GAC) near the A site, where eEF1A mediates translation elongation; increased binding of GCN2 in this region via RPLP0, enables GCN2 activation, which is prevented if eEF1A binds GCN2. Thus, GCN2 activation may indicate reduced eEF1A activity and elongation as seen in starvation-stress conditions. Altered elongation has been observed with the FXR1 paralogue, FMRP. Therefore, as our data showed that GCN2 associates with ribosomes in FXR1 OE cells but not in control vector cells (FIG. 3Q), and is activated due to RPLP0 that increases in FXR1 OE cells (FIG. 3B, M), we tested whether elongation may be altered in FXR1 OE and KD cells, compared to control cells.

We treated cells with harringtonine to block initiation followed by a time course to observe translated products from pre-existing elongating ribosomes, with puromycin termination and labeling (SunRiSE method). We observed a difference in labeling at the earliest time points, similar to the nascent global translation in FIG. 1F, indicating increased translation with FXR1 OE compared to control vector cells that is then blocked by harringtonine. We examined labeled products that were smaller in size, as these would reveal differences in elongation rates. The harringtonine time course shows moderately enhanced labeling with FXR1 KD compared to control shRNA cells (FIGS. 4H, 8H). FXR1 OE showed a slight but not significant trend of decreased labeling, compared to control vector cells. While additional studies are needed, these data hint that FXR1 depletion may moderately increase translation elongation. This is in addition to FXR1's role in increasing translation initiation of specific 5′UTR bearing mRNAs (FIGS. 3S-3T, 7J, GUG reporter, FIG. 4D, BCL6 5′ UTR reporter), rewiring translation for surviving such conditions.

FXR1-Overexpressing Cells Show Decreased Monocyte Migration, and Enhanced Survival with Macrophages

Our data show altered translation of immune regulators in FXR1-overexpressing cells. Based on our data, these cells may subvert immune cells by decreasing translation of immune susceptibility genes (receptors, immune cell-attracting chemokines), while also promoting translation of immune evasion genes such as CD47 that inhibit anti-tumor macrophages (FIG. 4C, up and down in translatome genes). Since FXR1-overexpressing cells show decreased translation of immune-attracting chemokines (FIG. 4C, down in translatome genes), we first tested whether FXR1 overexpression alters migration of monocytes. We find that co-cultured monocytes, in a trans-well assay, show 50% reduction in monocyte migration toward FXR1 OE cells compared to control vector cells (FIG. 5A). This is consistent with our data that show decreased translation of immune-attracting chemokines in FXR1-overexpressing cells (FIG. 4C, down in translatome genes), as well as our data that showed altered immune regulators in G0 and AraC-treated cells. These data indicate that monocytes are precluded from the environment around FXR1 overexpressing cells through decreased translation of immune-attracting chemokines, which could allow FXR1-overexpressing cells to evade associated anti-tumor immune activity.

Secondly, we find that immune evasion genes, elevated in the FXR1-overexpression translatome, are also increased in G0 and AraC-treated cells, and include SLAMF6, and CD47 that block anti-tumor immune activity. Therefore, we tested whether FXR1 OE cells would show increased survival when co-cultured with macrophages that can be evaded by CD47, compared to control vector cells. Flow cytometry analysis revealed greater survival of FXR1 OE cells (by 22%) compared to control vector cells, after co-culturing with macrophages (FIG. 5B). These data indicate that FXR1-overexpressing cells translate immune evasion regulators to survive anti-tumor immunity.

Chemoresistance of G0 and FXR1 Overexpressing Cells is Reduced by Drugs that Override eIF2α Phosphorylation, or Block Translated Pro-Survival Genes, XBP1 and BCL6

We targeted the eIF2α phosphorylation that is commonly observed in G0 and FXR1 overexpressing cells and is a potential vulnerability as such cells use non-canonical translation that would require eIF2α phosphorylation, to express genes for chemosurvival. We used ISRIB that is known to inhibit eIF2α phosphorylation, to first test whether the translatome observed with FXR1 overexpression can now be reversed. Western blot analysis revealed decrease of known non-canonical targets such as ATF4 that are not translated in the presence of ISRIB. As shown in FIGS. 5C-E, we performed polysome analysis in FXR1-ovexpression cells with or without ISRIB treatment followed by qPCR of mRNAs that are translationally increased in FXR1-overexpressing cells. We find that ISRIB reduced polysome association of several mRNAs that are upregulated in FXR1-overexpressing cells, without affecting other genes like vinculin. These data indicate that FXR1 overexpression promotes specific, pro-survival mRNA translation under eIF2α phosphorylation conditions. If so, then overriding this unconventional translation should reduce chemosurvival of FXR1-overexpressing cells. Consistently, we find that FXR1 OE cells that are treated with ISRIB and AraC have reduced chemosurvival compared to FXR1 OE cells that are treated with buffer and AraC. To confirm that the translated genes are important for survival in FXR1-overexpressing cells, chemosurvival was tested after inhibiting two known survival genes (XBP1, BCL6) that are upregulated in FXR1-overexpressing cells, compared to untreated, control vector cells.

As shown in FIG. 4F, we performed polysome analysis in FXR1-ovexpression cells with or without ISRIB treatment followed by qPCR of the above identified mRNAs; we find that ISRIB treatment reduced polysome association of mRNAs that are upregulated upon FXR1 overexpression (graph) (FIG. 4G). These data indicate that FXR1 overexpression promotes non-canonical translation of mRNAs needed for chemosurvival. If so, then overriding this non-canonical translation should reduce the chemosurvival of FXR1-overexpression cells. Consistently, we find that FXR1 overexpressing cells that are treated with both ISRIB and AraC have reduced chemosurvival. This was also observed in G0 cells that enhance FXR1 (FIG. 4H). Chemosurvival was also decreased upon treatment with small molecule inhibitors of target genes that are enhanced in G0 and AraC treated cells and in FXR1 overexpression cells compared to vector control cells: Toyocamycin (FIG. 4I), an inhibitor of XBP1, a pro-survival gene, as well as with FX1, an inhibitor of BCL6 pro-survival oncoprotein (FIG. 4J), that are enhanced in translation by FXR1 overexpression (FIGS. 4A-4DE). These data indicate that non-canonical translation regulated by FXR1 contributes to chemosurvival.

### SUMMARY

We found that G0 and chemotherapy treated cells demonstrate inhibition of canonical translation, along with eIF2α phosphorylation; this inhibits proliferation gene translation and permits non-canonical translation of specific genes by unknown mechanisms, which could mediate chemosurvival. Our data showed that FXR1 is increased in G0 THP1 cells of specific cancers such as AML. Since G0 cells are chemoresistant, this indicates that FXR1 could play a role in chemosurvival. In accord, we find that AraC chemotherapy treated cells transiently upregulate FXR1 (FIG. 1A). These data indicated that FXR1 would be necessary for chemosurvival. Consistently, we find that overexpression of FXR1 causes increased survival of THP1 cells with AraC chemotherapy, while knockdown of FXR1 reduces chemosurvival (FIGS. 1B-C). These data indicate that FXR1 mediates chemosurvival in conditions like G0 and chemo-treated THP1 cells where it is increased. We further find that in FXR1 depletion cells, nascent translation is shut down by 50% (FIG. 1D). Analysis of polysomes to investigate this effect revealed a sharp decrease of 40-60% of both ribosome subunits (FIG. 1E). These data indicate that FXR1 depletion causes dysregulation of ribosomes, indicating a role for increased FXR1 levels in G0 chemoresistant cells in ribosome regulation that may lead to specific translation changes for chemosurvival.

Interestingly, we find that FXR1 depletion reduces all rRNAs, including the precursors, many snoRNAs that increase in G0, and several RPs; conversely, FXR1 overexpression leads to their increase (FIG. 2A-C, 3A, 3C). Our data show that FXR1 regulates snoRNAs that modify rRNAs (snoRDs and snoRAs), many of which are involved in modifying critical sites on the ribosome. Consistent with the increase of snoRD63 and snoRA2A on FXR1 overexpression, we find increased modification at their rRNA target sites on 28S rRNA (FIG. 2D-2E); in accord, pseudouridylation is reduced upon FXR1 depletion along with alterations in other rRNA modifications, as observed by RNA mass spectrometry (FIGS. 2F, 6A). FXR1 depletion and overexpression also alters U3 and U8, which process rRNAs, explaining the alteration in rRNA levels (FIG. 2B). We also find that FXR1 can associate with snoRNA and ribosome biogenesis regulators such as NOLC1 (FIGS. 2G and 6C). Consistent with the fact that both Pol I transcribed rRNAs, as well as Pol II transcribed snoRNAs and RPs are affected by FXR1, we find that FXR1 interacts with DDX21 (FIG. 2H), a regulator that promotes Pol I and Pol II transcription of ribosome genes. In accord, we find that DDX21 overexpression partially rescues the reduced 45S rRNA levels in FXR1 knockdown cells relative to a control vector (FIG. 2EI). Given that all 4 rRNAs are affected, and the 45S rRNA precursor from Pol I transcription is also affected along with the Pol III transcript, 5S rRNA, our data indicated that FXR1 may be influencing a common rRNA transcription factor for Pol I and Pol III. Accordingly, we find that FXR1 associates with the mRNA of POLR1D, a transcription factor associated with and needed for both Pol I and Pol III transcription; consistently, FXR1 depletion decreases while overexpression increases POLR1D (FIGS. 2J-2L, 6D), which would impact all 4 rRNA expression levels (FIG. 2A). Together, these data reveal that FXR1 associates not only with ribosome components, but also with ribosome biogenesis regulators that deregulate ribosomes in accordance with changes in FXR1 levels, and thus FXR1 increase could lead to ribosome and translation changes in G0 chemoresistant cells.

Collectively, these data indicate changes in the ribosome. Consistently, we find that the ribosomes migrate differently, in two distinct complexes in G0 and AraC-chemoresistant cells compared to one in proliferating cells; this is also observed with FXR1 overexpression (FIG. 3B). We find that FXR1 overexpression leads to activation of GCN2 and PKR, which are eIF2α kinases (FIG. 3F, 3H). These kinases disable canonical translation via eIF2α phosphorylation, which permits non-canonical translation on specific mRNAs such as those with structured 5′ UTRs and altered start sites. We find that overexpression of FXR1-regulated snoRNAs, snoRD46 or snoRA2A, lead to increased eIF2α phosphorylation (FIG. 3G). Additionally, we find that P stalk proteins are altered in levels in FXR1 overexpression cells as in G0 chemoresistant cells (FIG. 3C-3D, FIG. 7C). Consistently, we find that overexpression of FXR1 that promotes such ribosome changes in G0 chemoresistant cells, shows increased GCN2 phosphorylation and activation and consequently eIF2α phosphorylation (FIG. 3I). P-stalk protein levels can induce GCN2 associated on the ribosome; consistently, in FXR1 overexpression cells where P0 increases along with GCN2 and eIF2α phosphorylation, we find increased association of GCN2 with ribosomes and with P0 (FIG. 3J). In accord, in FXR1 depleted cells, we find that GCN2 association with ribosomes and with P0 as well as eIF2α phosphorylation are decreased (FIGS. 3J-K). These data indicate that increased FXR1 in G0 chemoresistant cells alters the ribosome, which itself then can induce stress signals to inhibit canonical translation and permit non-canonical translation. With eIF2α phosphorylation, strict use of only conventional Kozak start sites for initiator tRNA recruitment is reduced and can lead to non-canonical start site translation. This could permit translation of specific mRNAs such as those with structured 5′UTRs and AUGs in poor Kozak start sites that would normally be poorly translated by canonical translation. In accordance with these ribosome and translation changes that should permit non-canonical translation, we find that G0 and chemosurviving cells where FXR1 is overexpressed and elicits these changes, as well as in FXR1 overexpressing cells, translation of a reporter with a non-canonical GUG start site is promoted compared to a reporter with a conventional AUG start site and compared to untreated serum-grown proliferating cells and control vector cells (FIG. 3L-M, 7E). Together, our data indicate that FXR1 increase in G0 chemoresistant cells alters translation toward non-canonical translation through a signaling role elicited by ribosome changes that lead to eIF2α phosphorylation and ISR.

Such inhibition of canonical translation can lead to expression of specific mRNAs that are critical under stress conditions and are usually not translated either due to the unconventional start site used or due to structured 5′ UTRs that are inhibiting for the canonical translation mechanism^{86,89,90,129,131,132}. Examining translated genes identified in G0 chemoresistant cells¹ revealed their enhanced polysome association in FXR1 overexpressing cells (FIGS. 4A-4D). These genes are known targets downstream of ISR stress signaling such as XBP1 or have high ΔG or a G-rich motif or structured 5′UTRs, such as NCAM1 and CD47 (FIG. 8); these indicate that non-canonical translation mechanisms would be needed for their translation, as with such 5′ UTRs. Consistently, treatment with ISRIB, an ISR inhibitor that overrides eIF2α phosphorylation to restore canonical translation, prevents this translation (FIGS. 4E-4F). These changes would also support the post-transcriptional and translational roles observed with FXR1 and in aggressive cancers where FXR1 is increased.

Thus, these data reveal that pro-survival genes with critical roles in tumor survival and progression, such as XBP1, NCAM1 and BCL6, are translated by these non-canonical ribosome changes that are triggered by FXR1 increase in G0 chemosurviving cells. This indicated that chemosurvival in FXR1 overexpressing cells as in G0 and chemoresistant cells, where FXR1 is increased, and causes such ribosome changes, could be reversed by targeting this non-canonical translation or such downstream translated genes. Consistently, we find that ISRIB inhibition of this non-canonical translation (FIG. 4H) or inhibition of the downstream target genes such as that of XBP1 with an inhibitor or of BCL6 (FIGS. 4I-4J) reduced the chemosurvival observed in G0 and FXR1 overexpression cells. Together, our data reveal that FXR1 is a critical ribosome regulator that is amplified in G0 chemosurviving leukemic cells to promote ribosome changes that trigger stress signals to enable non-canonical translation of pro-survival genes that lead to increased tumor persistence.

These findings confirm hidden layers of gene expression regulation, via altered use of critical RNAs, due to chemical and coding modifications that are induced by stress signals on RNAs in cancers. These mechanisms can be mapped to reveal hidden causes of persistent disease. Apart from the unique causes, the specific gene expression pattern can be targeted with combination therapies. Understanding such RNA changes led to effective RNA vaccines; thus, our studies can reveal critical insights and unique vulnerabilities that can be targeted without toxicity for effective therapies for patients with refractory diseases. This disclosure provides a resource of unique vulnerabilities, to use as a tool to develop new therapies for cancer and other disease.

We find chemical and RP (ribosomal protein) modifications on ribosomes and mRNAs in therapy-treated and in G0 leukemic cells. Non-canonical translation of modified mRNAs needs to be accommodated by ribosome changes. The ribosomal changes not only accommodate the mRNA changes and elicit their specialized translation, but also act as a stress signal. The ribosome changes trigger stress signals and translates non-canonical start sites to express new epitopes and survival genes. These are needed for drug and anti-tumor immunity survival; inhibiting the G0 ribosomal changes curbs tumor survival. Our data show that ribosomal proteins (RPs) are dysregulated in persistent cancer cells; these promote a specific translatome to drive tumor progression, which can be blocked with inhibitors. Together, our studies reveal that G0 or therapy stress induces specific genes, by altering RNAs and ribosomes, to elicit tumor survival.

The gene sets expressed include metabolic signaling genes, anti-apoptotic oncogenic BCL6, mTOR and PI3K related signaling genes, stem cell marker genes like CD93 that are associated with survival, cell adhesion genes like NCAM1 and PECAM1, which are known to promote drug survival, ER stress genes like XBP1, and many immune evasion genes such as CD47, HLA-G, HLA-E and other such receptors that inactivate anti-tumor immunity. When we synthesize mRNAs from these genes, which have (a) G-rich motifs in their 5′-UTRs that cause these regions to form structures which promote inefficient expression and (b) non-canonical start sites (e.g., poor Kozak consensus sequences, such as deviated from RCCRCCAUGG (SEQ ID NO: 98, where R is any purine), around the start sites or non-AUG start sites), on reporters to express luciferase (in place of a toxin to kill such cells), we can effectively enhance production of such reporters (see FIGS. 3L-3M, FIG. 8A). In contrast, the usual reporters and 5′-UTRs (that are used by the RNA industry) fail to produce in such conditions. Tagged onto a toxin or p53 or other suppressor would allow specificity to express only in diseased or targeted cells as the non-canonical reporter is suppressed in normal cells or cells that are not chemoresistant.

This specialization to avoid therapy and anti-tumor immunity of the chemoresistant or aggressive tumor cells is also its vulnerability, such that we can very specifically target not only the genes expressed but also use this mechanism to express RNAs as drugs exclusively in these cells, as is shown in FIGS. 3L-3M. Thus, such 5′-UTRs and non-canonical start sites (see FIGS. 3L-3M, 8A for example) can be employed to translate and suppress or kill chemoresistant cells. These RNAs that would express a toxin or apoptotic gene or a suppressor to suppress cell growth (genes like bacterial toxins that can kill the cells and are already in use, or simply to suppress growth with p53 expression or other such genes), would be expressed only in cells that would activate the eif2 phosphorylation pathway. Cells that do activate this pathway outside of early development (including stem cells, where it is a normal pathway) are under stress, as in disease, or are infected, especially by viruses, but not exclusively. Such diseases includes brain diseases (Costa-Mattioli et al., Science 368 (6489) eatt: 5314, 2020.) as well as aggressive cancers and conditions of stress in cancer (e.g., cancers treated with chemotherapeutic agents, such as cytarabine)—it's an escape mechanism for cancers and is a deregulation that is best studied in brain diseases, some immune related diseases that are inflammatory such as rheumatoid arthritis, and infections.

### Materials and Methods

The results described above were obtained using the following materials and methods.

### Cell Culture

THP1 cells were cultured in Dulbecco's modified Eagle medium (RPMI) 1460 media supplemented with 10% fetal bovine serum (FBS), 2 mM L-Glutamine, 100 μg/mL streptomycin and 100 U/ml penicillin at 37° C. in 5% CO₂. SS THP1 cells were prepared by washing with PBS followed by serum-starvation at a density of 2×10⁵ cells/mL and AraCS cells, by treatment with indicated concentrations of AraC for indicated periods of time. THP1 (TIB-202) and MV4:11 (CRL-9591) were obtained from ATCC. MOLM13 (ACC554) were obtained from DSMZ. Cell lines kindly provided by David Scadden.

### Plasmids

TRIPZ plasmids expressing shRNA against human FXR1, and miR30a primiR sequences used as control (RHS4750), were obtained from Open Biosystems and MGH cancer center, respectively. Stable cell lines were constructed as described by Open Biosystems. The stable cells expressing shRNA against FXR1 were induced with 1 μg/mL doxycycline for two days (once each day) to knockdown FXR1. Control cells were treated similarly.

THP1 FXR1 OE cell lines were created by infecting cells with an FXR1 containing retroviral vector for constitutive over expression of FXR1. Corresponding control cell lines were made by infection with empty vector.

Polysome Profiling with Microarray

Sucrose was dissolved in lysis buffer containing 100 mM KCl, 5 mM MgCl₂, 2 mM DTT and 10 mM Tris-HCl (pH 7.4). Sucrose gradients from 10% to 55% were prepared in ultracentrifuge tubes (Beckman). Harvested cells were rinsed with ice-cold PBS and resuspended in lysis buffer with 1% Triton X-100 and 40 U/mL murine (New England Biolabs) for 20 minutes with intermittent tapping on ice. After centrifugation of cell lysates at 12,000×g for 20 minutes, supernatants were loaded onto sucrose gradients followed by ultracentrifugation (Beckman Coulter Optima L90) at 32,500×rpm at 4° C. for 80 min in the SW40 rotor. Samples were separated by density gradient fractionation system (Biocomp Piston Gradient Fractionation). RNAs were purified by using TRIzol (Invitrogen) from heavy polysome fractions and whole cell lysates. The synthesized cDNA probes from WT Expression Kit (Ambion) were hybridized to Gene Chip Human Transcriptome Array 2.0 (Affymetrix) and analyzed by the Partners Healthcare Center for Personalized Genetic Medicine Microarray and BUMC facilities. Gene ontology analysis for differentially expressed translatome or proteome was conducted by DAVID 6.7 tools. Molecular signatures enriched in FXR1 OE and Control cells were identified by Gene Set Enrichment Analysis (GSEA).

### Western Blot Analysis

Cells were collected and resuspended in lysis buffer containing 40 mM Tris-HCl (pH 7.4), 6 mM MgCl₂, 150 mM NaCl, 0.1% NP-40, 1 mM DTT, 20 mM, 17.5 mM β-glycerophosphate, 5 mM NaF and protease inhibitors. Samples containing 80 μg of protein were loaded onto 4%-20% gradient SDS-PAGE (Bio-Rad) or 16% SDS-PAGE (Invitrogen), transferred to PVDF membranes and processed for immunoblotting.

Antibodies against FXR1 (used for Western), Actin, Tubulin were from Millipore; NOLC1, DDX21, PolR1D, RPLP0, RPLP2, RPL29, RPL11, FXR1 (used for Immunoprecipitation) were from Protein Tech; PKR, P—PKR, EIF2α, P-EIF2α, GCN2 (used for immunoprecipitation) were from Cell Signaling; P-GCN2, GCN2 (used for Western) were from abcam.

### Quantitative RT-PCR

Total RNA was isolated using TRIzol (Invitrogen) according to the manufacturer's instructions. The cDNA was synthesized from 1 μg of RNA using M-MuLV Reverse Transcriptase (NEB) and random hexamer primer (Promega). qPCRs were run on LightCycler® 480 Instrument II (Roche) using 2×SYBR green mix (Bio-rad).

### Mass Spectrometry for RNA Modification Analysis

Total RNA was isolated using Trizol as per manufacture's instructions. Isolated RNAs were cleaned using RNaseEasy kit (Qiagen). PolyA containing RNAs were separated from the RNA pool using a PolyAtract mRNA isolation system IV (Promega). The remaining unpolyadenylated RNA was sent for LC MS/MS analysis to Go Beyond RNA Arraystar, Inc. analysis service).

### Nascent Translation Assay

Cells were grown in methionine free RPMI. ³⁵S-methionine was added as a methionine source. Cells were washed with PBS and lysed in buffer (40 mM Tris-HCl (pH 7.4), 6 mM MgCl₂, 150 mM NaCl, 0.1% NP-40, 1 mM DTT and protease inhibitors). The lysate was first separated by electrophoresis on an SDS-PAGE gel, then transferred to a PVDF membrane by Western Blotting. Transcripts were visualized by autoradiography.

Low dNTP qPCR Assay

For analysis of 2′O-methylation, RNA was prepared from indicated cells. Reverse transcription (as described above in qPCR) was performed with primers in the reverse orientation from the modification site on ribosomal RNA. Two different dNTP concentrations were used for each primer, low (0.025 mM) and high (2.5 mM). qPCR was done with the resulting cDNA using primers at forward and reverse orientation to the modified sites. Fold change in modification was calculated based on difference of Ct values in the low and high dNTP conditions. For analyzing pseudouridylation, first the RNA was treated with CMCT (1-cyclohexyl-(2-morpholinoethyl) carbodiimide metho-p-toluene sulfonate) under alkaline conditions. Rest of the experiment was done as described above for 2′O-methylation.

### Immunoprecipitation

In vivo crosslinking was done using 0.3% formaldehyde. Cells were washed with cold PBS and pelleted. Cell pellets were resuspended in hypotonic buffer (10 mM Tris (pH=8), 1.5 mM MgCl₂, 10 mM KCl). Cells were lysed using a syringe with 25 G*5/8 precision glide needle. Cytoplasmic fraction was collected by centrifuging at 2000 rpm for 10 mins. The remaining pellet was resuspended in equal volumes of buffer (20 mM Tris (pH=8), 25% glycerol, 1.5 mM MgCl₂, 0.2 mM EDTA, 20 mM KCl) and buffer (20 mM Tris (pH=8), 25% glycerol, 1.5 mM MgCl₂, 0.2 mM EDTA, 1.2 mM KCl), incubated on cyclomixer for 45 min, sonicated 6 times, 30 secs each, centrifuged for 10 min at 2000 rpm. The supernatant was mixed with the cytoplasmic fraction. In some cases, when mentioned only cytoplasmic fraction was used. The lysates were then cleaned by incubating with ProteinG (Santa Cruz Biotechnology) beads and IgG. Post cleaning, lysates were incubated with antibodies and IgG as controls overnight in buffer (40 mM HEPES, 100 mM NaCl, 6 mM MgCl₂, 0.025% NP-40, 1 mM DTT, 10% glycerol, 1 mM PMSF). Lysates were then incubated with equilibrated and blocked ProteinG beads for 2 hr. Beads were then pelleted and washed 4 times with RIPA buffer. Beads were then either used to analyze proteins or RNA after uncrosslinking.

### DEAE (Diethylaminoethanol) Fractionation Assay

Cell lysates or fractions post Y10B immunoprecipitation, were incubated with equilibrated DEAE beads for 2 hr in buffer (40 mM HEPES, 6 mM MgCl₂, 2 mM DTT, 10% glycerol, 150 mM NaCl). After collection of the flowthrough, beads were incubated with wash buffer of increasing salt concentrations (40 mM HEPES, 6 mM MgCl₂, 2 mM DTT, 10% glycerol, 250 mM/500 mM/750 mM/1000 mM/2000 mM NaCl). RNA was isolated from each salt fraction. Amount of ribosomal RNA in each fraction was analyzed by qRT-PCR.

### Luciferase Assay

Plasmids containing firefly luciferase reporters downstream of AUG/GUG start sites were co-nucleofected with renilla luciferase in THP1, FXR1 OE and control cells. Nucleofected THP1 cells were then grown under conditions of S+, G0, and 5 UM AraC. Cells were pelleted, washed, and lysed in 1× passive lysis buffer. Luciferase activity in the lysates were analyzed using Luciferase Assay System (Promega) as per manufacture's instructions.

### Inhibitors

Cytarabine (AraC), Trans-ISRIB, FX-1, and Toyocamycin were obtained from Cayman Chemicals. For polysome analysis, FXR1 OE and control cells were treated with 1 μM trans-ISRIB for 24 h. For cell viability FXR1 OE and control cells were treated individually or with a combination of 5 μM AraC and 1 μM trans-ISRIB for 24 h; 100 nM Toyocamycin and 1 μM AraC; 10 μM FX-1 and 5 μM AraC.

### Statistical Analyses

All experiments in every figure used at least 3 biological replicates except for microarray, mass spectrometry, and patient sample data. Each experiment was repeated at least 3 times. No statistical method was used to pre-determine sample size. Sample sizes were estimated on the basis of availability and previous experiments. No samples were excluded from analyses. P values and statistical tests were conducted for each figure. Statistical analyses were conducted using R or Excel. Two-tailed unpaired t-test or Wilcoxon rank sum test was applied to assess statistical significance. SEM (standard error of mean) values are shown as error bars in all figures. Means were used as center values in box plots. P-values less than 0.05 were indicated with an asterisk. E-values were used for the statistical significance in the motif analysis.

### Sequences

The sequences described herein are represented as RNA sequences or as DNA sequences that represent RNA molecules (e.g., antisense RNAs, siRNAs, or shRNAs). These sequences may also be represented as corresponding RNA sequences. One skilled in the art, for example, would understand that the cDNA sequence is equivalent to the mRNA sequence, except for the substitution of uridines with thymidines, and can be used for the same purpose herein, for example, the generation of a shRNA for inhibiting the expression of FRX1 mRNA. Uridines can optionally be modified for stability according to standard methods.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the following numbered paragraphs.
1. A method of treating a refractory leukemia comprising administering a BCL6 inhibitor, a XBP1 inhibitor, a ISR inhibitor, a PI3K/mTOR or RPS6K inhibitor, a PDK inhibitor, a PKR inhibitor, a GCN2 inhibitor, or a shRNA to a patient alone or in combination with cytarabine or other chemotherapy.
2. The method of paragraph 1 wherein the BCL6 inhibitor is FX1.
3. The method of paragraph 1 wherein the XBP1 inhibitor is toyocamycin.
4. The method of paragraph 1 wherein the ISR inhibitor is selected from ISRIB and metformin.
5. The method of paragraph 1 wherein the P13K/mTOR or RPS6K inhibitor is selected from bez235, torin, pp242, AZD3147, everolimus, CAL-101 and LY2584702.
6. The method of paragraph 1 wherein the PDK inhibitor is AZD7545.
7. The method of paragraph 1 wherein the PKR inhibitor is c16.
8. The method of paragraph 1 wherein the GCN2 inhibitor is GZD824, A-92, or a triazolo[4,5-d]pyrimidine.
9. The method of paragraph 1 wherein the shRNA blocks the triggered eIF2 phosphorylation pathway.
10. The method of paragraph 9 wherein the shRNA is an antisense sequence selected from:

ATCCGTCTCCACAGACAAG;
TGTTTTGCAAATAAAACTG;
TGAGCATGTTCAGCAGCGT;
TGGACTTCCAGGTCGCCCT;
and
TGCGCATCATGGTGTTCTT.

11. A method of treating a chemoresistant cancer comprising administering a BCL6 inhibitor, a XBP1 inhibitor, a ISR inhibitor, a PI3K/mTOR inhibitor, a PDK inhibitor, A PKR inhibitor, a GCN2 inhibitor, or a shRNA to a patient alone or in combination with cytarabine or other chemotherapy.
12. The method of paragraph 11 wherein the BCL6 inhibitor is FX1.
13. The method of paragraph 11 wherein the XBP1 inhibitor is toyocamycin.
14. The method of paragraph 11 wherein the ISR inhibitor is selected from ISRIB and metformin.
15. The method of paragraph 11 wherein the P13K/mTOR inhibitor or RPS6K inhibitor is selected from bez235, torin, pp242, AZD3147, everolimus, CAL-101 and LY2584702. The method of paragraph 11 wherein the PDK inhibitor is AZD7545.
16. The method of paragraph 11 wherein the PKR inhibitor is c16.
17. The method of paragraph 11 wherein the GCN2 inhibitor is GZD824, A-92, or a triazolo[4,5-d]pyrimidine.
18. The method of paragraph 11 wherein the shRNA blocks the triggered eIF2 phosphorylation pathway.
19. The method of paragraph 19 wherein the shRNA is an antisense sequence selected from:

ATCCGTCTCCACAGACAAG;
TGTTTTGCAAATAAAACTG;
TGAGCATGTTCAGCAGCGT;
TGGACTTCCAGGTCGCCCT;
and
TGCGCATCATGGTGTTCTT.

20. The method of paragraphs 11 through 20 wherein the chemoresistant cancer is selected from Acute myeloid leukemia, lung cancer, breast cancer, ovarian cancer, head and neck cancer, esophageal cancer, cervical cancer, uterine cancer, stomach cancer, bladder cancer, metastatic prostate cancer, urothelial cancer, and other cancers that have the 3q-26 locus chromosomal amplification where FXR1 is located, including but not limited to pancreatic cancers, and gliomas.

1. An isolated nucleic acid molecule comprising a G-rich 5′ untranslated region (UTR) operably linked to a heterologous transgene.

2. The nucleic acid molecule of claim 1, wherein the 5′-UTR comprises motif 1 (SEQ ID NO: 13): [symbol] [symbol] [symbol] [symbol] [symbol]
GCR¹CR²R³CR³R³R³R²R³CR³CR³R²R³CR²CCR³CR²GCR²R³CCR²ACCCCR³R³GCR¹CGC wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside; and
each R³, independently, denotes a guanosine or cytidine nucleoside.

3. The nucleic acid molecule of claim 1, wherein the 5′-UTR comprises motif 2 (SEQ ID NO: 14): [symbol] [symbol] [symbol] [symbol] [symbol]
R¹CR²CR³R²R³GR⁴R²CR²GR⁵R²GCR²GCGGCGGCGR³R²GGCTGR²GGCGGCGGCGR⁴R³G
wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside;
each R³, independently, denotes a guanosine or cytidine nucleoside;
each R⁴, independently, denotes a cytidine, guanosine, or adenosine nucleoside; and
each R⁵, independently, denotes a cytidine or adenosine nucleoside.

4. The nucleic acid molecule of claim 1, wherein the 5′-UTR comprises motif 3 (SEQ ID NO: 15): [symbol] [symbol] [symbol] [symbol]
GCCR³R²R¹GCCGR³CR⁴CCR³CCGCR¹
wherein each G denotes a guanosine nucleoside;
each C denotes a cytidine nucleoside;
each A denotes an adenosine nucleoside;
each R¹, independently, denotes a cytidine or thymidine nucleoside;
each R², independently, denotes a cytidine, thymidine, guanosine, or adenosine nucleoside;
each R³, independently, denotes a guanosine or cytidine nucleoside; and
each R⁴, independently, denotes a thymidine or guanosine nucleoside.

5. The nucleic acid molecule of claim 1, further comprising a non-canonical translation start site.

6. The nucleic acid molecule of claim 5, wherein the non-canonical translation start site comprises a poor Kozak consensus sequence or a non-AUG start site.

7. The nucleic acid molecule of claim 6, wherein the non-canonical translation start site comprises a codon selected from AUA, CUG, GUG, CUG, ACG, and AUC.

8. The nucleic acid molecule of claim 1, wherein the nucleic acid molecule is RNA.

9. The nucleic acid molecule of claim 8, wherein the RNA is messenger RNA (mRNA).

10. The nucleic acid molecule of claim 1, further comprising a 3′ UTR.

11. The nucleic acid molecule of claim 1, further comprising a 3′ poly-adenosine (polyA) tail.

12. The nucleic acid molecule of claim 11, wherein the polyA tail comprises from about 10 to about 100 continuous adenosine residues.

13. The nucleic acid molecule of claim 1, further comprising a 5′ cap.

14. The nucleic acid molecule of claim 1, comprising a poly-A tail.

15. The nucleic acid molecule of claim 1, wherein the nucleic acid molecule comprises a chemical modification.

16. The nucleic acid molecule of claim 15, wherein the chemical modification comprises a modified nucleoside.

17. The nucleic acid molecule of claim 16, wherein the modified nucleoside is N6-methyladenosine.

18. The nucleic acid molecule of claim 1, wherein the nucleic acid molecule is operably linked to a polypeptide.

19. A pharmaceutical composition comprising the nucleic acid molecule of claim 1 and one or more pharmaceutically acceptable carriers, diluents, and/or excipients, optionally wherein the pharmaceutical composition comprises an exosome, liposome, or lipid nanoparticle (LNP).

20. A composition comprising an LNP and the nucleic acid molecule of claim 1.

21.-85. (canceled)


SEQUENCE LISTING (98 sequences)

SEQ ID NO: 1 - DNA, 19 bp, synthetic construct
  source 1..19: mol_type = other DNA
atccgtctccacagacaag

SEQ ID NO: 2 - DNA, 19 bp, synthetic construct
  source 1..19: mol_type = other DNA
tgttttgcaaataaaactg

SEQ ID NO: 3 - DNA, 19 bp, synthetic construct
  source 1..19: mol_type = other DNA
tgagcatgttcagcagcgt

SEQ ID NO: 4 - DNA, 19 bp, synthetic construct
  source 1..19: mol_type = other DNA
tggacttccaggtcgccct

SEQ ID NO: 5 - DNA, 19 bp, synthetic construct
  source 1..19: mol_type = other DNA
tgcgcatcatggtgttctt

SEQ ID NO: 6 - DNA, 46 bp, synthetic construct
  source 1..46: mol_type = other DNA
tactggccttggtttaattctcgagaattaaaccaaggccagtagc

SEQ ID NO: 7 - RNA, 25 nt, synthetic construct
  source 1..25: mol_type = other RNA
ctatgagacccttacgtgattgtta

SEQ ID NO: 8 - RNA, 25 nt, synthetic construct
  source 1..25: mol_type = other RNA
gcacatgcatcttctgtatggacaa

SEQ ID NO: 9 - RNA, 19 nt, synthetic construct
  source 1..19: mol_type = other RNA
gcatggccctcttctgatt

SEQ ID NO: 10 - RNA, 19 nt, synthetic construct
  source 1..19: mol_type = other RNA
gtacagcgattggattaac

SEQ ID NO: 11 - RNA, 19 nt, synthetic construct
  source 1..19: mol_type = other RNA
cagagaaggtgaaacgatc

SEQ ID NO: 12 - RNA, 19 nt, synthetic construct
  source 1..19: mol_type = other RNA
taactgaagtgaagtgatg

SEQ ID NO: 13 - DNA, 45 bp, synthetic construct
  source 1..45: mol_type = other DNA
gcycnscsssnscscsnscnccscngcnsccnaccccssgcycgc

SEQ ID NO: 14 - DNA, 48 bp, synthetic construct
  source 1..48: mol_type = other DNA
ycncsnsgvncngmngcngcggcggcgsnggctgnggcggcggcgvsg

SEQ ID NO: 15 - DNA, 21 bp, synthetic construct
  source 1..21: mol_type = other DNA
gccsnygccgscvccsccgcy

SEQ ID NO: 16 - DNA, 461 bp, synthetic construct
  source 1..461: mol_type = other DNA
acaagcgagctggtggttgaagctggttaaagaacagcctaggtattccagaagtgtttgaggatcccttccatgaaggaagagaggaaagtttttaagtaaacctcccactcccatgtgtcttcagctttcttttgcaaaggagaaaatccttgaagtttggtaaagaccgagttagtctatctctctttgcctatctcgagttgggctggggagaggaggagataggttcttttgtctttttctgtcttctcccttccccacttccttccctccagtccccactcactcacatgcacacactaaccttggagccgatgggattgagtgactggcacttgggaccacagagaaatgtcagagtgtttggttacagactcaaggaaacctctcattttagagtgctcatttggttttgagcaaaattttggactgtgaagcaaggcattggtgaagacaaa

SEQ ID NO: 17 - DNA, 603 bp, synthetic construct
  source 1..603: mol_type = other DNA
atgcgcggggcgggagtgagcgaaattcaagctccaaactctaagctccaagctccaagctccaagctccaagctccaaactcccgccggggtaactggaacccaatccgagggtcatggaggcatcccgaaggtttccggaagccgaggccttgagcccagagcaggctgctcattacctaagggtcttgctgtgtcgcccagactggaattcagtggcctgatcatagttcactgcagcctcgaactcctgggctcaagcagtcctcctgccccagcctccctagtagctgggacttaagatatgtgaaagaggccaaagaagcaactaagaatggagacctggaagaagcatttaaacttttcaatttggcaaaggacatttttcccaatgaaaaagtgctgagcagaatccaaaaaatacaggaagccttggaggagttggcagaacagggagatgatgaatttacagatgtgtgcaactctggcttgctactttatcgagaactgcacaaccaactctttgagcaccagaaggaaggcatagctttcctctatagcctgtatagggatggaagaaaaggtggtatattggctgatgat

SEQ ID NO: 18 - DNA, 45 bp, synthetic construct
  source 1..45: mol_type = other DNA
gagtgcgagctgaaagctgctggagagtgagcagccctagcaggg

SEQ ID NO: 19 - DNA, 195 bp, synthetic construct
  source 1..195: mol_type = other DNA
ggtttccagccatggctgccattacctgaccagcgccacagccggtctctctgcaggcgccgggagaagtgaccagagcaatttctgcttttcacagggcgggtttctcaacggtgacttgtgggcagtgccttctgctgagcgagtcatggcccgaaggcagaactaactgtgcctgcagtcttcactctcagg

SEQ ID NO: 20 - DNA, 148 bp, synthetic construct
  source 1..148: mol_type = other DNA
gcccagccggtcaccgcctgccggctgcagacgcctgcgagcaggttgtttttataagaggcgtcattggcgcccgagctgtgaccgccgccactggggcagccagcacaatcgggcggaggtggcgctgccccttcagacctgaaag

SEQ ID NO: 21 - DNA, 456 bp, synthetic construct
  source 1..456: mol_type = other DNA
gctgtgtggctgtttctgtacactcactggcaggcttggtgccggctccctcgcccgcccgcccgccagcctgggaaagtgggttacagagcgaaggagctcagctcagacactggcagaggagcatccagtcacagagagaccaaacaagaaccctttcctttggcttcctcttcagctcttccagagggcttgctatttgcactctctcttttgaaattgtgttgcttttacttttcacccttctgcttgggttttatgagggctttgttaagtcttagagggaaaagagactgagcgagggaaagagagaggcaaagtggaaaggaccataaactggcaaagcccgctctgcgctcgctgtggatgaaagccccgtgttggtgaagcctctcctcgcgagcagcgcgcacccctccagagcaccccgcggacccgcacctcggcgtggccacc

SEQ ID NO: 22 - DNA, 126 bp, synthetic construct
  source 1..126: mol_type = other DNA
gcacgcgcggttgggagcctcgcccatgctttgtcgcgttaccggggctaccgtttgcgcccccgactgcaacggtggccgctgagggaggaggcttctcgaatacggtttctgtcttcgcgttaa

SEQ ID NO: 23 - DNA, 171 bp, synthetic construct
  source 1..171: mol_type = other DNA
agttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagct

SEQ ID NO: 24 - DNA, 99 bp, synthetic construct
  source 1..99: mol_type = other DNA
atttcagggcgtagcaggcggaagtaagggtgagaggaggctgcaacgccgagcggaggaggcaggaaccggagcgcgagcagtagctgggtgggcacc

SEQ ID NO: 25 - DNA, 157 bp, synthetic construct
  source 1..157: mol_type = other DNA
gcggggaagtggcggctgaagcgccgccggcggggctcactgtggtggtgtgagtgggaggcggcggcgctggtggctgcagctggggtgaggcgcgaggcggcgcactcgacggctgactggagcagcggatataagtcaaagagtacagcagaaa

SEQ ID NO: 26 - DNA, 11 bp, synthetic construct
  source 1..11: mol_type = other DNA
acagtgctggg

SEQ ID NO: 27 - DNA, 456 bp, synthetic construct
  source 1..456: mol_type = other DNA
aggagcggcgagctagcggacgcaaagactgggcatgctccgcggcggcgcaggttttggtcacaagtaggaagaagccagtgcaccagaccggcaaagagaagcgggagccgccgcggcagcgcggccgtggggtccgccgccgccgcatcggagcgggaggaggagcagcggggagggcgaggccgccgggccgagagccgtcccgcctgctctcggtcttctgccttcgcctccgcgcggtgcgtcggacccagggtctgtcacctgggcgccaggggccgccgccggggagccggagcgggcaggaccctccctccgccgactgcggcccgagagcgcccccgcggggtggagcggcagccgccttctgcgggcggctgagtgtccgtctcgcgcccggagcgggcgaccgccgtcagcccggaggaggaggaggaggaggagggggcggcc

SEQ ID NO: 28 - DNA, 661 bp, synthetic construct
  source 1..661: mol_type = other DNA
agtgcgtgcgtgagcgagtgagcgaaaaggagagcgcgcggcggacgcgcccgggacgcgcggcggcaccgggaggccgggccgagcggttttgagagattacctgaagttgcactcaaaagcaagatatactactaactttgaaatggaataaggccctccctatcctgaagacattagctgagagtctggcaccttttaaaggtctgatcatcaattgtctctctgggcggccacctatgagacttcatctgtgtgctttataaatagcagtccagggcaacagtacttgataaaaaggttataccttattggtgtggacgttgtctaaatttcggtagccatggcacaagaatataagaaagcatgggattatggcaaccacagaatctcagtagtacaagttccattcagttttttctgaaagaaagccctctgttaaagtgaagcaaagaaactgttgtggattataacgtttagaagttccaatttttcagtgctttacaaataaagcatcatttaaccttttaaatgaaaaagattaagatctcatgcaactgttgtattttctggaagccattctccaaaagggaagtgcacatttaaaacacagatatgatggtccttgctgcagggatttaagtctacttgcttttacatc

SEQ ID NO: 29 - DNA, 21 bp, synthetic construct
  source 1..21: mol_type = other DNA
agacaaggttttccaagcaag

SEQ ID NO: 30 - DNA, 170 bp, synthetic construct
  source 1..170: mol_type = other DNA
gctgtcctggagctgctgctgctgctgcggcggctgcaccggcggcgccgaggccgagatcgaggccggggtgcgcgcttcgcaaacgtgccctatccgtgcggcttggctgcgccagcccttgcggccacccgggcgtctaggcgggtctgtgcgccgcccgggcgagg

SEQ ID NO: 31 - DNA, 182 bp, synthetic construct
  source 1..182: mol_type = other DNA
gcttcccttccacgtctcccggaagcggtagcagggccccggcgggtcggggaggaggtttactcagcttgggccccctccgggccagccgccgagggggcgcggcccaggacggcggctaggccgtagtgcagcctctccggagtcctcaggtttgccaataggattatcctgctgccatc

SEQ ID NO: 32 - DNA, 744 bp, synthetic construct
  source 1..744: mol_type = other DNA
ggcggcggtggcggccgaggaggagaacatggcggccgcggagagcggctgaaatgcctgttcttcaggccgggcgagcgggagtctgacgcgatttgctgagctctgtcctccgacggctgcctcgcggccttgagggcctccgccgcctgttcgctgctgctccctcgagcggagcctgcgtcaggttccctctgcatccccctgcggggggacccctgctccggaggagggggccggagagccgcggcgccgggcccgaggagcggcggaggccggggcggcgccgccgccacggtcgcggacgagtgcggcgccggtgagcggggcccagaggcggcggcggcagatctatccatactaatggaaatgtaactggaactgactctgatgataaaatcaaggaccatcaagcaagatcatgcagtaggcaactttgcttccaaaagaagttaccaacatttagaatttctacttattctgaggttccagttacagctatatcagagaatgagttaatctcctcagaaatcactaaatactactctgaggggcttagaaattaacaggttgtttatataattggccttaaatgaggtgagagtgaagagactagagccatctctggaaaacatcattatcccattccccgggaagctaccctctggaactcaagatttgaccatatctgttttgaggattcattatgaacaaagaagtctcccaggtgtgaagtttttcaac

SEQ ID NO: 33 - DNA, 106 bp, synthetic construct
  source 1..106: mol_type = other DNA
acaaccagctgactcccgtagaggaagacactgtggaggccagttctggagctattgcagcctcggttgcccggccggggacccgagccgaaaagttatcgtcaga

SEQ ID NO: 34 - DNA, 487 bp, synthetic construct
  source 1..487: mol_type = other DNA
actctccaagagactgctggcgccggcgcccgcccgcgcggcaccgacgcggggagcgcgcttcgcgctgactcagcggcggcggcggctgcggcggcggcggcggcgttagccggccctcgcgctctttcccttcctggggcgccgaccccgcccgcttgcttgcttgcttgcttgcctgcctgcctgcctgcccggcgccacgcaagagaaggtgccaggggacgcagagcgactagaggcgcgcggtcccggccagcaccgtctctggcgttgtagctgcggccgtggcggaggactacggcgacaaggacgagggccgctctcccagctctctgcgtgccgcgccgctccgctccgctggctgaccatctggagtgcaggctgggaggcgggatggagtgatagggaagatgtttataaattcttctgtgggatcagagggcacgcctattacaaccagaaaactacaagtataacagcgagg

SEQ ID NO: 35 - DNA, 50 bp, synthetic construct
  source 1..50: mol_type = other DNA
gctatggtaaccgaggaggccggggcccgggtgtggtatttttggagtta

SEQ ID NO: 36 - DNA, 61 bp, synthetic construct
  source 1..61: mol_type = other DNA
gtttccgaagggaccaccgggaacagacggatcggcagggcggggcggaacggcgtttgca

SEQ ID NO: 37 - DNA, 308 bp, synthetic construct
  source 1..308: mol_type = other DNA
ggcagtgggagcagcggcagcagcttcggctgctgctttcaggctgccgctgcattaggggcttcctgaggaagcgcgggcggacgacagaggatgccgaaccactccagtcatgactgtccaaagtatgataatcacatgagagtgctcgttgctacggatgtcatttgactcatcagagaaaatctgtctaaaagaaaatatccatgtgaccaaatccatttcattattgaatggcttgatggatttcctttactctgattcataccaaagctgtccttctcaaccaaagcaagaaaggatcctgc

SEQ ID NO: 38 - DNA, 222 bp, synthetic construct
  source 1..222: mol_type = other DNA
gccccacccgcgagccgcagccgcggccccacagcttctggggctggggccccggcagccgggcaggccggcctgacctcgacctccgccgtgcgggcccgaccggtgagtccagcccggcagtcgcaggacccggccgccagcctctccctccacctctccctgcccccagcgccaggcgcgggctgcgctcggtggcggcggcggggccctcaggcggcc

SEQ ID NO: 39 - DNA, 22 bp, synthetic construct
  source 1..22: mol_type = other DNA
agtcgggagaacccatccagac

SEQ ID NO: 40 - DNA, 167 bp, synthetic construct
  source 1..167: mol_type = other DNA
ggaggcggcgttgccgggctctccggaaggagacgtggcggcggttgggccggtgatacccgggcgctttatagtcccgccgcctcctcctccacctcctcctcctcctcctctcctcctggagcagaggaggttgtggcggtggctggagaaagcggcggcggagg

SEQ ID NO: 41 - DNA, 123 bp, synthetic construct
  source 1..123: mol_type = other DNA
gcagcctgggcagtgggtcctgcctgtgacgcgcggcggcggtcggtcctgcctgtaacggcggcggcggctgctgctccggacacctgcggcggcggcggcgaccccgcggcgggcgcggag

SEQ ID NO: 42 - DNA, 376 bp, synthetic construct
  source 1..376: mol_type = other DNA
gggctcgggcgccggccgggcttgcgcagcgctaggctcctcgccgctcctagtctgcggcggcggcggcggggagggcgcggccgcgggcggggctgagggcggcgggggcgggccgcccgagctgggagggcggcggcgccgaggggaggagagcggcccatggacccgcggggcccggcgccccagactctgcgccgtcgggacggagcccaagatgtcggcctaggccggggcgcgacgacgcggacggggcggcgaggaggcgccgctgctgccggggctcgcagccgccgagcccccgagggcgcgccctgacggactggccgagccggcggtgagaggccggcgcgtcgggagcgggccgcgcggcacc

SEQ ID NO: 43 - DNA, 1294 bp, synthetic construct
  source 1..1294: mol_type = other DNA
ccgggccgcgcgctgaggagccgcccggtcgcctgcgcgctccctccggcggcgtccccagcccgcggcccctctgctgccggcccccggctcgccggctgcgggagtggcctcaagatggatgaagacggcggcggcgagggtggtggcgtgcctgaagacctctcattagaagagagagaagaacttctagacattcgtcgaagaaaaaaggaacttattgatgacattgagaggctgaaatatgaaattgcagaggtgatgacagagatcgacaatctaacttccgtagaggagagcaaaacgactcagaggaacaaacagatagccatgggaagaaagaaattcaacatggatcccaaaaagggaattcagtttctaatagaaaatgacctgctacagagttccccagaagacgtcgcccagttcctttataaaggagaaggcctaaataagaccgtcattggggactacctgggtgaaagggatgaatttaatattaaagttcttcaagcctttgttgaactccatgagtttgctgatctcaaccttgtacaagccttaaggtaagtatgatcatttgctgggtgtagagtggatggggaaggccagaggaccactgcctgggcagcagtggcggtgaattgactccaggtaacttggtgccacacgagtctcacagatgctggctccttttccggatgatccagctaatgctgtgtgacactgagcagccctagacaggaattggggcacacgcttaagataagggccgtgctttcctcactgtggccgctgatgctccttccattccgtttccttttatttgtaatggtgtatggcagtagtcgagtgccttgttcccaagctctgtatggcgtggcgttggtgctccatggggttacttccttccacacttgacccaaagcaccaaacgcatggatacctgagtgtgccagagaggatgtaggtgtctcgccattttggtgtctgggacacactccctggatgcccccttgagctgcccttcctgcacgttgtcttcctcctgctctgcaagcaggaagttgtctcgcttccttccttggtgccctggctgagtcgcgtgggctcttcatctgttctgggccttgaccctgagccagcactcagcagcatccgggggagaggaggcagggcttctggtgggagtcgcctcagccggcccttggactaagaaaatcaggccgcacctcgtcatcttctcaaatcttcggcttcataccttgttctcgtttacgtggactgtttgctc

SEQ ID NO: 44 - DNA, 339 bp, synthetic construct
  source 1..339: mol_type = other DNA
agtggtagcggttattcggcggcccgcggcggaccatggccctggccctggcccggcgtcgctgggctttcctcacggcgtccccgagcagcgtcgcagagcgggccgacttccgggaaggaactgaccagcgactgagcggcggccggcgcgcttagcgccctgaacatgcggcagtccctgcgggcgaccccgggctccggacaggcggcggcggaggcggcggctcgggagggaaggaggcggcggcgccggcggaggtggcggcggagacggccggcgcccggcgcggagccctagggaggcagttcagcgcggcctcgggcctcgtcgagaagg

SEQ ID NO: 45 - DNA, 95 bp, synthetic construct
  source 1..95: mol_type = other DNA
gacaccgcacgctgagctctgtgatgtagccgcttgcggagactgcaagcagccgcggcgcgcccggccctccctcttccgctgccgccgtggga

SEQ ID NO: 46 - DNA, 185 bp, synthetic construct
  source 1..185: mol_type = other DNA
atatctgctgctgccgccgcagttgcgaatgcagcatcggcgcttagctgcctccgcggtgcagctaaggttcgtgtcgctaccccttggcccttcgctcttgctgccttaaccccgccggcggagcccgctcttctggcctgttgagcccgctccctcactgccacacagcaagttccgagacc

SEQ ID NO: 47 - DNA, 839 bp, synthetic construct
  source 1..839: mol_type = other DNA
agtcctgggcgcgcacaaacccggaagcggatcgcgtggagtgaaggtcctaccacggcgcgtgagttttgctctgccttggattaagtctgcacttcccaggtccccggcgcttctgcccctgggacgtgggatccccacggacctggaaattctcgcctgtcttcccttcacccagagcaaattgagacgtcccggaggaagaccaaggcagcctattgggccttccaggcaatcacatgggaatcagccacacgtccttcctcctcacctcagaacatctcagaataacttggtgaaatgtctcccactgtgagcctcagtgagcccacctgtaacatagaggcctcgcccctgagctctacaatcctgtgtccagttgtctcctcagctgtctcctgggtcatcaaacaggcatccccaccttcaggtgtccacgagtggctttctaaacccccaaacacatttccttgcagtctgcacatctcagatgagggtgactacgtacttccggaaacggccgaacttgacagcatgtattttaaatttgtgaaataaattactttatttgtaagtgttgtaatttataatataaagagaaacttagatgtatacgtgaaaagagtgagaagatacatcacttccaatttttgtttgtttgtttgtttttttgagaggaattttcactcttgtggctgaggctggagtgcaatgccatgatatcagctcactgcaacctctgactcctgggatcaagggattctccttcctcagactcccgagtagctgggatcacagtcgactttcaaaattctttaaggattgattcctaaagactc

SEQ ID NO: 48 - DNA, 154 bp, synthetic construct
  source 1..154: mol_type = other DNA
agtgcggacgtcgccattcctggcccatgggaagattgcgtttcacctgctcctgaaggccgaaggtggctctagcgcatcctttgtcgcgccgtgacctgcaggtactgacagatccgtagggaggacaccgtgacttcccggacgctgggaa

SEQ ID NO: 49 - DNA, 10 bp, synthetic construct
  source 1..10: mol_type = other DNA
gggtggcaag

SEQ ID NO: 50 - DNA, 46 bp, synthetic construct
  source 1..46: mol_type = other DNA
ctttcccgggcgctgattcctgagtgctgagcgcgaacccgaggag

SEQ ID NO: 51 - DNA, 754 bp, synthetic construct
  source 1..754: mol_type = other DNA
ctcccactgccgttgtgggtaacgcggacgtggaagaacctcgtctgcggaggaaaaggtagatgttaaatggtaactacgcgcgaggttctgaggagccctgggaacaggaaggagaaaagaataccaaaagtgacaacagtttgccaatcgcagtctttaatctgataaagcggttatctcgtcttgagtcccaggtgccgagtcaatccccatacacagccgccgccattgcctcgagtccttgtgtctgactgtctgttcctgctgctgtatgacacagcacctcgaggcaaggaaataagaaaactgcctctgatccaagcagagaaggtagatctgtccccaggtctgtggaactgtcagttgtgaagttttgtaaaatggtcacccaacttaaaactaggaaattacgaagaagagaaaattgccctgtatctgttaaggtctgcctgtagatctgctgtagggcttgtcaccattggaagcaaggtcctacttcagtggcagatctggtggccttggagtggctgaagaccaccaccctccacagggctgggcccatgcacagccatccttccctaccttgagtgagcttcctctgcatgttttctatatcactggcagagcctgtagttggaaaggggacagagtgactactggactttgtgtgaaaacaccaaccgggacaaaacttcagtcaaggctgagacgggtgggggtatataacttgtccttacgttaaacttggaac

SEQ ID NO: 52 - DNA, 51 bp, synthetic construct
  source 1..51: mol_type = other DNA
acttcctctcaagcttggcgtttgtttggtggggttacacgcgggttcaac

SEQ ID NO: 53 - DNA, 108 bp, synthetic construct
  source 1..108: mol_type = other DNA
agagggaatgagtgtgagctcgtgagtgggcgccgccgccaccgcccccgccgccgtcgtctcggtagcagccttcgccacgccggggtcttcagctccactggggcc

SEQ ID NO: 54 - DNA, 38 bp, synthetic construct
  source 1..38: mol_type = other DNA
agtccgggtttggagactcctgcgtcctccgacttttc

SEQ ID NO: 55 - DNA, 186 bp, synthetic construct
  source 1..186: mol_type = other DNA
agatttccctccttcgtcgctgttgctgccgccatacgcgctctccctgtttagctcttctgttagaaatagtatctttgttttcctttgctgttcctcaatcccctactcttcaccccttgttttcacctattttgcgagaacccatccagatcccccttcccttcttcccctgccggcccagtt

SEQ ID NO: 56 - DNA, 139 bp, synthetic construct
  source 1..139: mol_type = other DNA
gatttgtggtcccggagaagagattggcggaggggaaagactgagtcgtctttcctttgcccgcttctgtggactgttcgaacgcccaggggtgggccaaacccgtttcttaggaaaccaggaatccagggaaggcaga

SEQ ID NO: 57 - DNA, 257 bp, synthetic construct
  source 1..257: mol_type = other DNA
agttacatgctggtacggtaacctgaggaggcgaggactgattcttggtgtgggggcgggttctaggtacatttaaagctttctggaatgggcggagcctggggcaagacaaattaagggaggatatgggaggaggagcctaagtctgggcggttcttgaatttagatttgcttttcccagcggggaagggaccggatctgaaaggagatgctctctgattcctaaaagggtgggggctggctgggcgcggtggcgc

SEQ ID NO: 58 - DNA, 319 bp, synthetic construct
  source 1..319: mol_type = other DNA
cctctgcgagctggcttaggaggagctgttttgcatcccttcacgtcagccctgcctcattcccttcttccagggagggagagagcgcgagcgagagagcgagcgagaggaggggggagagagagaaagagaggagccggagggagggcggcaggagcaggcggcgggcgcgcgtggaggcgggcggcgggcgcacagcagcagcccgggcggtgggcagccaggagcccccggcccggcccggcccggcccgccgagggccccagcgcaggagccgcgcccggacccaggcgatgccctcctagctagccgtcacggg

SEQ ID NO: 59 - DNA, 59 bp, synthetic construct
  source 1..59: mol_type = other DNA
ctgcccagacttgaagccacacaggcaggtcgggcaggcgggtcgcaggttgtaaatcc

SEQ ID NO: 60 - DNA, 83 bp, synthetic construct
  source 1..83: mol_type = other DNA
aggctttctgacccggaatggaggaggcggaggagctgctcttggaggggaagaaggcgctgcaactcggtgagtgacctgcg

SEQ ID NO: 61 - DNA, 112 bp, synthetic construct
  source 1..112: mol_type = other DNA
actctttcctgtcccggcctgcgtggtgtgggcttgtgggtctttgagacccgaaaattgagagcgttttcgcactccagcggctgctcctggcggctctgcggccgtcacc

SEQ ID NO: 62 - DNA, 99 bp, synthetic construct
  source 1..99: mol_type = other DNA
gtctgccgcctcagcctcagccccaacctcagccgccgccgttgcgcttgctcccgggcggtcctggcctgtgccgccgccgcccccagcgtcggagcc

SEQ ID NO: 63 - DNA, 77 bp, synthetic construct
  source 1..77: mol_type = other DNA
agttgcactgtagcctcggcagtgaaccgggaggtactaccaggtcgccgtcgttgctccgctcgctctgagagagc

SEQ ID NO: 64 - DNA, 75 bp, synthetic construct
  source 1..75: mol_type = other DNA
ccttttcccaccccctagcgccgctgggcctgcaggtctctgtcgagcagcggacgccggtctctgttccgcagg

SEQ ID NO: 65 - DNA, 350 bp, synthetic construct
  source 1..350: mol_type = other DNA
cccagaccagcagaggcagcagccggagcagccgcagcctgcgccctctcccgcccgcccgccctccgcccgcccgcccgccctccgccgccctccacccgccccggggtctctttcccccttcctcctcctcctcctccacccccccttcctcctccgcccgcccgcggggcccccctcgccttcccgcccgcccctattgttccgcccccggcctcccgcccttccccttcccgcccgctccccttttcccctcagtcgcctcgcgcctgcagtttttggctttcacccccaaccagtgaccaaagacttgaccactcaaagtccagctccccagaacactgctcgac

SEQ ID NO: 66 - DNA, 12 bp, synthetic construct
  source 1..12: mol_type = other DNA
cttccttgtaag

SEQ ID NO: 67 - DNA, 41 bp, synthetic construct
  source 1..41: mol_type = other DNA
agcttccggcacggccttcaagcgcgggacgcgacaaagtc

SEQ ID NO: 68 - DNA, 127 bp, synthetic construct
  source 1..127: mol_type = other DNA
agaggccacgcccccggaagcggcggtgcaggtacgaaaagcgcgcgcggggattccaggagtcgtttcttgccttgatgtactggagcaatcagatcacacggcggcttggagaaacccagggacc

SEQ ID NO: 69 - DNA, 82 bp, synthetic construct
  source 1..82: mol_type = other DNA
atttggagtcaggcctggctgttgctcaggtgaccagcttgtgtctctgggagggcgctgctttccccggccacccggcgcg

SEQ ID NO: 70 - DNA, 51 bp, synthetic construct
  source 1..51: mol_type = other DNA
gactctgggaggcgtttgggcccagagaagtggatccgccgcttgcgccgc

SEQ ID NO: 71 - DNA, 21 bp, synthetic construct
  source 1..21: mol_type = other DNA
agcggagacctcgctgcgctc

SEQ ID NO: 72 - DNA, 121 bp, synthetic construct
  source 1..121: mol_type = other DNA
agactcctgcggatgccattatgaggagcaagaacgcgaaggggcctcgggtccgctaggccgggatccggagccgcccgaagccggtgccgcagccccctgcgcccccggtgcccccgac

SEQ ID NO: 73 - DNA, 361 bp, synthetic construct
  source 1..361: mol_type = other DNA
gggtgatcctcttatgcctgtggccttggcctctgaggacggcagtgagtgctgttccctcagtacggtggaggcttcaagggcctcctagctgctgtgcctctgctgacatttggcaggcagcttctgccagccaagggtctcaccccagccacccggctctgcacccactgtgctgcccacaggagtggtcctgcccatggaagggccagttcaggtggccggagctcctgagctgccctaggggactgctgtgggtctgaggtggtgatgtcccccacggctgcctgcgcctgagcccccacgcatccacccctggggccactctgctgttcaggagcacccacccgtgtcctcgacc

SEQ ID NO: 74 - DNA, 35 bp, synthetic construct
  source 1..35: mol_type = other DNA
gcgtctcttgtttgtgcggctgaccagttggcgac

SEQ ID NO: 75 - DNA, 25 bp, synthetic construct
  source 1..25: mol_type = other DNA
aacggacgaattaagcggcgtggag

SEQ ID NO: 76 - DNA, 32 bp, synthetic construct
  source 1..32: mol_type = other DNA
actcgtctgcgacggcgccttcgcgaaacact

SEQ ID NO: 77 - DNA, 158 bp, synthetic construct
  source 1..158: mol_type = other DNA
acttcctccgcggttcctcggagccgcctcgctcctcttcagggactttgctgagaagggctctcgggcgtccagaccccaccgcaaaggtgtttggcgatccgccgagaagttgttggccccaggagcatccctcggggccgaatgcgcagtggacg

SEQ ID NO: 78 - DNA, 49 bp, synthetic construct
  source 1..49: mol_type = other DNA
agttgaaccgctcgcgaggagggttgctagtggagaagcaaccttggcc

SEQ ID NO: 79 - DNA, 204 bp, synthetic construct
  source 1..204: mol_type = other DNA
ctcttcagttgtctgctactcagaggaaggggcggttggtgcggcctccattgttcgtgttttaaggcgccatgaggggtgacagaggccgtggtcgtggtgggcgctttggttccagaggaggcccaggaggagggttcaggccctttgtaccacatatcccatttgacttctatttgttgtttacttctggtttagtgtgaa

SEQ ID NO: 80 - DNA, 247 bp, synthetic construct
  source 1..247: mol_type = other DNA
gtgacgcaagggcggggttgccggaagaagtggcgaagttacttttgagggtatttgagtagcggcggtgtgtcaggggctaaagaggaggacgaagaaaagcagagcaagggaacccagggcaacaggagtagttcactccgcgagaggccgtccacgagacccccgcgcgcagccatgagccccgccccccgctgttgcttggagaggggcgggacctggagagaggctgctccgtgaccccacc

SEQ ID NO: 81 - DNA, 163 bp, synthetic construct
  source 1..163: mol_type = other DNA
gggcgcggcgtgtggcccttgcgggcgagcctggcggggttgcgttcctgtccgctagcaggtcaatggccagcaaggaggggggtccgagccggcggcggcggcggcagtggtggcagcgggagacaaatggaaacctccacagggcacagactccatcaag

SEQ ID NO: 82 - DNA, 1160 bp, synthetic construct
  source 1..1160: mol_type = other DNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacg

SEQ ID NO: 83 - DNA, 231 bp, synthetic construct
  source 1..231: mol_type = other DNA
aacacaaactttccgtcccgctcgctccctcctccgcgctcggcgcctcccgctccagcccggctcattccgcacattccggccagccccctccccacgaccccccttccccggccccccttgcggctccctcgggcccggcggagcggcccggccggagcgcccccgcgagctcggaccaggctcagccgcccagtgggctcaggcccagagcccagagcaaccagcaca

SEQ ID NO: 84 - DNA, 198 bp, synthetic construct
  source 1..198: mol_type = other DNA
gagtccctgcttcgcagagcaccggacttggagtcagagagacctggctgcgaatcccggctccgccactttctagcgctgtgaccttggttttcaggtggcgaaactgactggtctggttagagcgcgtccaagccaagccgcagtgctgtttggatttttaaatttctccttctgagtgagaaaatagcaatcgag

SEQ ID NO: 85 - DNA, 1286 bp, synthetic construct
  source 1..1286: mol_type = other DNA
agtttgccatgatggatgccagaacctgcacgtggggcttggtgacgcgatgactgagatgctggagtccccggtcagaaggacgatgccgagcgcgccgtattttctcctgcctctcctcagatcgccgccgttgttgggctgtggagacgtctcaaccaaagtggagccgacagaggccccgggagccgtggtgacagagcggccacaaccactagggggcgggaggcagagcggcccctagtccgcccctagtccgggacctctgggcggtcccgggcgggaagcgcacgcgcaaacgcggggcttcgcggccgctgtaaacaccaaagagcgcctgcgcaagccgagagtctcgggatcgacacgtgggggcgcctgagcgaagataaccgtaataaatagtaacctaacggtccagtcatcgttctgtggtcctttcttttatgattcacaaggaatgaccctcttcatcgcctctcctaattcagtcctcacaacagtccttttacaaatgggacaacaggttagaggaagtcaggcagatttccagcatcatagagagtaaaggaccagggaaggatcaggattcaaggactgcacccaggctctgcttccagcttgctgtgtgactttgggtaattttgttcccttagggaactgagctttctcatttgtaaatgcaaacaggctgttgggaggatcaaatgagatccaggggtgaaaacagcttagtttactttcaggaatttacccacgcggtatataaaggcaaaatattattatagtcaggtgattgtagattgaggaacccatttcctcattctgcaaattgcaaacctgagggcccaaagagggacaggggcttgccccaggtctcagcaggctgtgagcaagagctaaagcctaatcctcctgcctttgggcctggagcccttccttgtaccccaggggtcagtgtctttgttggatacaggcttagattgactgactgtaccctgagaacctaggggagtccctgttcccaattcttctcctacccccaccttggcctgatggaggaagaccctgctgtgttgagatgagcaccagagccaagaagctgaggaggatctggagaattctggaggaagaggagagtgttgctggagctgtacagaccctgcttctcaggtcccaggaaggtggcgtcagcatctgcagccgcgtcgacgttgtcggagcctccgcggaggacccaggagagccggactaggaccagggccctgggcctccccacactcccc

SEQ ID NO: 86 - DNA, 119 bp, synthetic construct
  source 1..119: mol_type = other DNA
aagtgtgccggcgtcgcgcgaaggttcagcagggagccgtgggccgggcgcgccggttcccggcacgtgtctcggcacgtggcagcgcgcctggccctgggcttggaggcgccggcgcc

SEQ ID NO: 87 - DNA, 496 bp, synthetic construct
  source 1..496: mol_type = other DNA
gcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcg

SEQ ID NO: 88 - DNA, 308 bp, synthetic construct
  source 1..308: mol_type = other DNA
gccagcagtgagtcggagctctatggaggtggcagcgggtaccgagtggcggctgcagcagcgactcctctgagctgagtttgaggccgtccccgactccttcctcccccttccctcccccttttttttgttttccgttcccctttcccctcccttccctatccccgacgaccggatcctgaggaggcagctgcggtggcagctgctgagttctcggtgaaggtatttcatttctcctgtcccctcccctccccaccccatctattaatattattcttttgaagattcttcgttgtcaagccgccaaa

SEQ ID NO: 89 - DNA, 117 bp, synthetic construct
  source 1..117: mol_type = other DNA
ggctcgcctccagcctcctgcgctccggtacctgggcgtcccaactccactgcgcgcccaaacccagccgagccggttcgtggcccgccccgccgggcggccgtcgacgcgagcgcc

SEQ ID NO: 90 - DNA, 384 bp, synthetic construct
  source 1..384: mol_type = other DNA
cttaacccttcctccgccggaacccaggctcggaaggtgagcaggggtgagcgcagccactgcccaacgcaaaccgtgaagaagcttctggaagagcagaggcgccgccagcagcagcagcccgacgctggcggggtgcagggacaatttctccctcccccagagcagcccctgaccccatctgtgaatgaggctgtgactgcacactcggagactgtgggttctggacctagcagcctgggctttccagactgggaccccaacacgcatgctgcctacactgacagcccctactcttgccctgcttctgctgccgaaaatttcctgcctcctgacttctacccaccctcggacccagggcagccgtgcccatttccccagggc

SEQ ID NO: 91 - DNA, 692 bp, synthetic construct
  source 1..692: mol_type = other DNA
attaggcagcagctgggggaagaatcaacacaccagggagcagagcggagcggatccgaggacagaggcggcagctgcctccgctgccgcccgccgccgccgccgccgcagccactgccggggctcagcccagcggcgggccccgcgcccccgcagccgccccgcggcgccccccggcgcgacccccgggccgcgcaccccgagctgcctccgggaggggggggggaaagggcccggatcctccttctccccctcctcctcctccccctcctctcaccctcaccctcaccctcctaccctgaccccctccccgcctccccggcccccctcaccccgaccctctccccggcccccgcctcccctcccccgcctcgccccaccggcttcccaccacggcctctctcggcgaggaaactctggcctccgcttcctcctcctccgactcggacaccggcggagcctccccgcccccgcggaagaaaccccgagcctcggcggcggagggagtaggagagcccggggcttcggcaggcagagcaggcctctcccctccgtcctcgtcgtcctcgtcctcctcctcctcctcctcctccgtggtggtagtggtgggacttcccccggctgctgcccctcccgccgccgctgctgtcccccaccgaagtagcggccacagcctggtcagcggcagcatc

SEQ ID NO: 92 - DNA, 107 bp, synthetic construct
  source 1..107: mol_type = other DNA
gttggcgtcttcgtcctgttgctggtctccgtccggtcgccggccgtctaggtctccggccctccccagccgctcctgcgcccttgccggccccgccgcccgcagcc

SEQ ID NO: 93 - DNA, 631 bp, synthetic construct
  source 1..631: mol_type = other DNA
agccgtcctgccggactgctggaggcggccacagcgccatgttggatgctctgctcgttgagtgaagaaaatccaccggcatcgcctgagccccgctaccgagaagggcgccgcttcctccggggagggggataaagatcccccgccgccggcccatgaggatattgccgtgaaaggcacagcgactgcagcaggaaccggacccggcaccggagcggcggcggcggcggcagcagcggtaccgcctcctcacccggcggcggcagcagcggcggcggcggcggcggcggcggcggcggcggcagcggtcccccctcctcacccgaacatcagggccctccagactcaggcgccccaacaaattcctagaggacctgtgcaacaacctcttgaggatcgaatcttcactcccgctgtctcagcagtctacagcacggtaacacaagtggcaagacagccgggaacccctaccccatccccttattcagcacatgaaataaacaaggggcatccaaatcttgcggcaacgcccccgggacatgcatcgtcccctggactctctcaaaccccttatccctctggacagaatgcaggtccaaccacgctggtataccctcaaacccctcagaca

SEQ ID NO: 94 - DNA, 365 bp, synthetic construct
  source 1..365: mol_type = other DNA
agcccccggcccggacccggagggatgcggagtggcggccgcggcggtggcggagactgtggctttaagagcgtgccgggagcccgagccccagccgggccgcgcttcgccgctgcgcaccccagcggagccaagccccacgctggccggacagggccgcctgtcgccgggctgctgagaactagccctagacctctgcgtgagggttcttctgccgaagacatcaccagtgtgtggagcctgccacacccacccgctgccaaaccacggcctttacctgtgtcttccggtgtttcccgtgcgacccatcctgtgggagtgcctcgtgggctgccccagagttcaccccacactcagcagcacca

SEQ ID NO: 95 - DNA, 1119 bp, synthetic construct
  source 1..1119: mol_type = other DNA
agtccctatttggaactgctctcgcggcagttcagacctcgtgctcgtccccttcgcctgtctgtgtgtggtatccgtaggtccggggcacttttttttttggtgtgtgtgtgtgtgcgtgcgcgcgtgtgtgtgtgttgggtgtgtgtgttttgggggtttgccggggcgcggagaggagagccgggccgaggggaaggaaggaagccgtagagctccccggtctcgggctgcgtctcccctccagcgctctcctgcctgtgcgctccgacgcggcagcggcggtggcgctgagcgctcctcggtggggacgcggtgctcaagctgggagcagcgagaaccctttgcggaggcttcccgtgcccgctggacttaggatgccagggcgtttgatttgcacctactgagcccggagttcctccccggccctgggaagggcttgcagcggcggcggcggcggcggcagcggcagtagcagacggaacagcaggctctcctctccgagggggggcgtcgaagcgcccgcgccgggctcccgccctcaccctccggcgctcctgcggccccaagccaccacctcgtagcccgcctccttcctcttctcgccgcgagccccaggcccggacgcctctaggatcggacccgcgcgccccggatgcttggggctgccccggcgcgcccccctacaggcgcgacccgctgagtgacccggagggggcccgagctcgccccaggaggggcccccggcctcgggaccccgcgccggacctgctggggagccgacgatgtccgcgcagtcccaggcattgacgcgctcgacgcagcccagggaaggcggccggccgctgcccgccgagccctaggcgcacaaagcccgcgcccgccgcccggccccggcgcccgccgacgactttgccgcctgctccgcggctctttgtctccacttggggcgggcgcccgactctgggatttcgctgcgagaacgagctgggggggccgggggcgagctctcggtttcccggccgccccccgctcgggctcggctcccccctcccccgcacctccccggcccgggctctcggcgcttccacgctctcggaatcacgacccctccctgcc

SEQ ID NO: 96 - DNA, 57 bp, synthetic construct
  source 1..57: mol_type = other DNA
gtgcggtccgcgcttttgtcccgctggcggccgggttcccgcggcgccgccacagcc

SEQ ID NO: 97 - DNA, 94 bp, synthetic construct
  source 1..94: mol_type = other DNA
actccaggtgtcgcccacttaactgaagatccaggatcccctccttaaggtgcccctggccggcagcgccgccgtggtcccggagcgcggcgac

SEQ ID NO: 98 - RNA, 10 nt, synthetic construct
  source 1..10: mol_type = other RNA
rccrccatgg